(19) United States
(12) Patent Application Publication
(10) Pub. No.: US 2025/0295648 A1
(43) Pub. Date: Sep. 25, 2025

(54) METHOD FOR TREATING L-DOPA-INDUCED DYSKINESIA, PARKINSON'S DISEASE, OR PARKINSON'S DISEASE MOTOR DISABILITY USING BEFIRADOL

(71) Applicant: Neurolixis, Inc., Park Ridge, NJ (US)

(72) Inventors: Adrian Newman-Tancredi, Castres (FR); Mark Andrew Varney, Park Ridge, NJ (US)

(21) Appl. No.: 19/083,674

(22) Filed: Mar. 19, 2025

Related U.S. Application Data
(60) Provisional application No. 63/567,329, filed on Mar. 19, 2024.

(51) Int. Cl. A61K 31/4545; A61K 9/48; A61K 31/198; A61P 25/14

(52) U.S. Cl. CPC A61K 31/4545; A61K 9/485; A61K 9/4866; A61K 31/198; A61P 25/14

(57) ABSTRACT

The disclosure provides a method for treating for treating L-DOPA-induced dyskinesia, Parkinson's disease, or Parkinson's disease motor disability in a patient in need thereof comprising daily administering to the patient a therapeutically effective daily dosage of L-DOPA and befiradol or a pharmaceutically acceptable salt thereof.

This application claims the benefit of priority of U.S. Provisional Application No. 63/567,329, filed Mar. 19, 2024, the contents of which are incorporated by reference as if written herein in their entirety.

### FIELD

This disclosure relates to a method for treating L-DOPA-induced dyskinesia in a patient in need thereof using befiradol or a pharmaceutically acceptable salt thereof. This disclosure further relates to a method for treating Parkinson's disease in a patient in need thereof using befiradol or a pharmaceutically acceptable salt thereof. This disclosure further relates to a method for treating Parkinson's disease motor disability in a patient in need thereof using befiradol or a pharmaceutically acceptable salt thereof.

### BACKGROUND

Parkinson's disease (PD) is characterized by a loss of nigrostriatal dopaminergic neurons resulting in the characteristic parkinsonian motor symptoms of tremor, rigidity, slowness of movement, and difficulty with walking. Treatment of these parkinsonian motor symptoms relies primarily on the gold-standard medication, levodopa (L-3,4-dihydroxyphenylalanine; L-DOPA), but over time the disease progresses, more and more neurons die, and the buffering capacity of dopamine is reduced.

As a result, a sizeable proportion of patients develop dose-limiting involuntary, hyperkinetic, non-rhythmic movements that are purposeless and unpredictable (dyskinesia), including chorea, dystonia, and athetosis. In fact, up to 50 percent of patients with Parkinson's experience L-DOPA-induced dyskinesia (LID) within five years of starting treatment with L-DOPA. LID affects about 200,000 patients in the United States, with motor complications (dyskinesia and OFF) worse during the day and sleep disturbed at night. The symptoms can become increasingly troublesome, with patients struggling to balance levodopa-induced dyskinesia with OFF time due to under-dosing of levodopa, and experiencing pain, frustration, embarrassment, isolation, risk of injury, social withdrawal, depression, poor quality of life, and increased care burden and costs.

Unfortunately, treatment of LID has been hampered by a lack of sufficiently efficacious and well-tolerated medications, although some success has been achieved by use of the glutamate receptor antagonist amantadine. However, its use, like that of other glutamate NMDA receptor antagonists, is limited by tolerance issues and side effects (e.g., hallucinations, confusion, leg edema, orthostatic hypotension) and suboptimal efficacy in some patients (e.g., about ⅓ of patients are non-responders, and anti-LID efficacy may decrease over time, especially in patients with severe PD). A clear need therefore exists for novel pharmacological approaches.

Several 5-HT_{1A} receptor agonists, such as buspirone, tandospirone, sarizotan and eltoprazine, have been clinically tested to alleviate LID and found to have limited efficacy. Moreover, use of these agents for LID can result in a worsening of parkinsonian symptoms, diminishing the important benefits of L-DOPA treatment (see e.g., Ludwig C. L. et al., “Buspirone, Parkinson's disease, and the locus ceruleus,” Clin Neuropharmacol, 1986, 9, 373-378; Politis M. et al., “Serotonergic mechanisms responsible for levodopa-induced dyskinesias in Parkinson's disease patients,” J Clin Invest, 2014, 124, 1340-9; Svenningsson P. et al., “Eltoprazine counteracts 1-DOPA-induced dyskinesias in Parkinson's disease: a dose-finding study,” Brain, 2015, 138 (Pt 4), 963-973). For example, a phase 2b study of sarizotan in Parkinson's patients with troublesome L-DOPA-induced dyskinesia found that two of the three tested doses significantly reduced L-DOPA efficacy (Goetz, C. G. et al., “Sarizotan as a Treatment for Dyskinesias in Parkinson's Disease: A Double-Blind Placebo-Controlled Trial,” Movement Disorders 2007, 22, 179-186); when the third (and lowest) dose was tested in two phase 3 clinical trials, it was found to be ineffective in reducing LID and further development was terminated (Rascol, O. et al., “A large phase III study to evaluate the safety and efficacy of sarizotan in the treatment of l-dopa-induced dyskinesia associated with Parkinson's disease: the Paddy-1 study,” Movement Disorders 2006, 21, S492-S493; Müller, T. et al., “The PADDY-2 study: the evaluation of sarizotan for treatment-associated dyskinesia in Parkinson's disease patients,” Movement Disorders 2006, 21, S591; Merck KGaA news release Jun. 23, 2006). The question remains, therefore, whether a selective and highly potent 5-HT_{1A} receptor agonist can show efficacious anti-LID properties, reducing undesired involuntary movement, without interfering with the beneficial anti-parkinsonian effects of L-DOPA, maintaining desired voluntary movement.

Befiradol, [NLX-112; (3-chloro-4-fluoro-phenyl)-[4-fluoro-4-{[(5-methyl-pyridin-2-ylmethyl)-amino]-methyl}piperidin-1-yl]methanone], is a selective and potent serotonin 5-HTIA receptor agonist (Colpaert et al., Neuropharmacology 2002, 43, 945-958; Newman-Tancredi A. et al. “Distinctive in vitro signal transduction profile of NLX-112, a potent and efficacious serotonin 5-HT1A receptor agonist,” J Pharm Pharmacol, 2017, 69, 1178-1190; Newman-Tancredi A. et al., “Translating biased agonists from molecules to medications: Serotonin 5-HT1A receptor functional selectivity for CNS disorders,” Pharmacol Ther 2022, 229, 107937), discovered by Pierre Fabre Laboratories (U.S. Pat. Nos. 6,020,345; 7,208,603). The structural formula of befiradol is shown below:

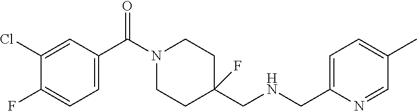 [SMILES: Cc1ccc(CNCC2(F)CCN(C(=O)c3ccc(F)c(Cl)c3)CC2)nc1]

Befiradol has demonstrated potential utility for the treatment of L-DOPA-induced dyskinesia in animal models, but its LID efficacy has heretofore not been tested in human clinical trials and there was concern that blood concentrations of befiradol higher than 15 ng/ml could be associated with side effects, such as dizziness, that would be unacceptable in relation to patients with movement disorders (WO 2016/005527). There was also concern that NLX-112 could not provide efficacious anti-LID properties without interfering with the beneficial anti-parkinsonian effects of L-DOPA.

Thus, there is a great need to find improved treatments for Parkinson's disease, and especially for treatments that reduce LID without causing either unacceptable side effects or a reduction in the beneficial anti-parkinsonian effects of L-DOPA. The present disclosure addresses this need and provides additional treatment advantages.

### SUMMARY

The present disclosure provides a method for treating L-DOPA-induced dyskinesia, Parkinson's disease, or Parkinson's disease motor disability in a patient in need thereof, comprising administering to the patient a therapeutically effective daily dosage of L-DOPA and befiradol or a pharmaceutically acceptable salt thereof as described herein.

In some embodiments, the therapeutically effective daily dosage of befiradol or a pharmaceutically acceptable salt thereof is administered transdermally. In some embodiments, the therapeutically effective daily dosage of befiradol or a pharmaceutically acceptable salt thereof is administered orally.

In some embodiments, the method comprises a titration phase followed by a treatment phase. In some embodiments,
a. at the beginning of the titration phase the befiradol or a pharmaceutically acceptable salt thereof is administered at an initial daily dosage that is lower than a therapeutically effective daily dosage of befiradol or a pharmaceutically acceptable salt thereof;
b. during the titration phase the initial daily dosage of befiradol or a pharmaceutically acceptable salt thereof is gradually increased to a therapeutically effective daily dosage of befiradol or a pharmaceutically acceptable salt thereof; and
c. during the treatment phase a therapeutically effective daily dosage of L-DOPA and befiradol or a pharmaceutically acceptable salt thereof is administered daily.

In some embodiments, a therapeutically effective daily dosage of befiradol or a pharmaceutically acceptable salt thereof is administered each day in one or more daily doses. In some embodiments, each such dose, if administered once to at least 6 elderly male subjects and at least 6 elderly female subjects in a clinical trial without a befiradol daily dosage up-titration period, provides a mean maximum plasma concentration of befiradol greater than about 15 ng/mL.

In some embodiments, a therapeutically effective daily dosage of befiradol or a pharmaceutically acceptable salt thereof is administered each day in one daily oral dose.

Preferably, the method of treating L-DOPA-induced dyskinesia does not significantly worsen the patient's parkinsonian symptoms. In some embodiments, the method of treating L-DOPA-induced dyskinesia significantly improves the patient's parkinsonian symptoms.

Preferably, the method of treating L-DOPA-induced dyskinesia does not significantly worsen the patient's parkinsonian motor symptoms. In some embodiments, the method of treating L-DOPA-induced dyskinesia significantly improves the patient's parkinsonian motor symptoms.

In some embodiments, if at least 15 subjects with L-DOPA-induced dyskinesia are treated with a therapeutically effective daily dosage of L-DOPA and befiradol or a pharmaceutically acceptable salt thereof according to a method of the present disclosure (subgroup 1) and at least 7 subjects with L-DOPA-induced dyskinesia are similarly treated with placebo and a therapeutically effective daily dosage of L-DOPA (subgroup 2) in a double blind, placebo-controlled clinical trial, the safety profile (e.g., proportion of subjects in each subgroup that experience at least one adverse event; the number of adverse events per subject in each subgroup; the incidence of a particular adverse event, such as dizziness, in each subgroup; the severity of adverse events) of the two subgroups is not significantly different.

These and other methods of treatment are described in more detail in the following description. References contained herein are hereby incorporated by reference in their entireties.

### BRIEF DESCRIPTION OF THE DRAWINGS

FIG. 1 depicts the effect of NLX-112 (NLX) and placebo (PBO) on UDysRS scores assessed during the phase 2a study described in Example 1 in the Per-Protocol Set at Baseline, at the end of Up-Titration (day 28) and at the end of the Stable Dosing period (day 42). Upper graph=total scores; lower graph=change in UDysRS total score from baseline. Baseline=visit 2, day 1; Titration=visit 6, day 28; Steady State=visit 7, day 42. *p<0.05 vs baseline; **p<0.01 vs baseline; n.s. not significant.

FIG. 2 depicts the effect of NLX-112 (NLX) and placebo (PBO) on UDysRS scores assessed during the phase 2a study described in Example 1 in the Per-Protocol Set at Baseline, at the end of Up-Titration (day 28) and at the end of the Stable Dosing period (day 42). Upper graph=total objective scores for parts 3+4; lower graph=change in UDysRS parts 3+4 score from baseline. Baseline=visit 2, day 1; Titration=visit 6, day 28; Steady State=visit 7, day 42. **p<0.01 vs baseline; n.s. not significant.

FIG. 3 depicts the effect of NLX-112 (NLX) and placebo (PBO) on UPDRS total score (upper graph) and the change in UPDRS total score from baseline (lower graph) during the phase 2a study described in Example 1. Baseline=visit 2, day 1; Titration=visit 6, day 28; Steady State=visit 7, day 42. **p<0.01 vs baseline; ***p<0.001 vs baseline; n.s. not significant.

FIG. 4 depicts the effect of NLX-112 (NLX) and placebo (PB) on UPDRS Part 3 score (upper graph) and the change in UPDRS Part 3 score from baseline (lower graph) during the phase 2a study described in Example 1. Baseline=visit 2, day 1; Titration=visit 6, day 28; Steady State=visit 7, day 42. **p<0.01 vs baseline; #p<0.05 vs PBO; n.s. not significant.

FIG. 5 depicts the effect of NLX-112 (upper graph) and placebo (PBO) (lower graph) on CGI-C score at end of steady state (visit 7, day 42) compared to baseline (visit 2, day 1).

### DETAILED DESCRIPTION

### I. Definitions

The term “about” is intended to qualify a numerical value as including a margin of measurement accuracy, such as a standard error of a mean value. In some embodiments, “about” a numerical value means the numerical value±10%.

The term “daily dosage” refers to the total amount of a drug that is administered to a patient in one day.

The term “elderly” refers to at least 65 years old.

The term “female” refers to an adult human woman.

The term “male” refers to an adult human man.

The term “patient” refers to an adult human being with Parkinson's disease or both Parkinson's disease and L-DOPA-induced dyskinesia.

The term “pharmaceutically acceptable” refers to those compounds, materials, compositions, and/or dosage forms which are, within the scope of sound judgment, suitable for use in contact with the tissues of human beings with acceptable toxicity, irritation, allergic response, and other problems or complications commensurate with a reasonable benefit/risk ratio.

The term “pharmaceutical composition” refers to the combination of an active ingredient with a pharmaceutically acceptable excipient, in a form suitable for administration to a patient.

The term “safe and well tolerated” refers to a drug treatment method having an acceptable safety and tolerability profile in patients treated according to the method, as assessed by parameters such as AEs, ECGs, vital signs, safety laboratory parameters, and physical examinations, consistent with the safety and tolerability requirements of government agencies responsible for regulating drugs for human use, such as the U.S. Food and Drug Administration (FDA) and European Medicines Agency (EMA).

The term “subject” refers to an adult human being participating in a clinical trial.

The term “sustained release” refers to a pharmaceutical composition that contains befiradol or a pharmaceutically acceptable salt thereof that, after administration to a patient, provides a time to reach maximum befiradol blood plasma concentration (Tmax) that is delayed by at least about one hour compared to the Tmax of a conventional immediate release tablet containing befiradol or pharmaceutically acceptable salt thereof.

The term “therapeutically effective” refers to effective to treat L-DOPA-induced dyskinesia, Parkinson's disease, or Parkinson's disease motor disability in a patient or patient population.

The term “treating” or “treatment” refers to delaying onset, slowing progression, stabilizing, or improving L-DOPA induced dyskinesia, Parkinson's disease, Parkinson's disease motor disability, or a symptom thereof. For example, treatment may include improvement of motor symptoms of Parkinson's disease. As another example, treatment may include delaying or preventing onset or development of L-DOPA-induced dyskinesia, such as by prophylactic treatment of a Parkinson's disease patient or population.

Throughout the description, where compositions are described as having, including, or comprising specific components, or where methods are described as having, including, or comprising specific steps, it is contemplated that, additionally, there are compositions of the present disclosure that consist of or consist essentially of the recited components, and that there are methods according to the present disclosure that consist of or consist essentially of the recited steps.

### II. Therapeutic Methods

### A. L-DOPA-Induced Dyskinesia

The present disclosure provides a method for treating L-DOPA-induced dyskinesia in a patient in need thereof, comprising daily administering to the patient a therapeutically effective daily dosage of L-DOPA and befiradol or a pharmaceutically acceptable salt thereof, wherein:
a. the method is safe and well tolerated;
b. the therapeutically effective daily dosage of befiradol or a pharmaceutically acceptable salt thereof provides, after at least two weeks of treatment according to the method, a mean maximum befiradol blood plasma concentration of at least about 30 ng/ml; and
c. the therapeutically effective daily dosage of befiradol or a pharmaceutically acceptable salt thereof is administered in a sustained release pharmaceutical composition.

The present disclosure further provides a method for treating L-DOPA-induced dyskinesia in a patient in need thereof, comprising daily administering to the patient a therapeutically effective daily dosage of L-DOPA and befiradol or a pharmaceutically acceptable salt thereof, wherein:
a. the method is safe and well tolerated;
b. the therapeutically effective daily dosage of befiradol or a pharmaceutically acceptable salt thereof provides, after at least two weeks of treatment according to the method, a mean maximum befiradol blood plasma concentration of at least about 30 ng/mL; and
c. the therapeutically effective daily dosage of L-DOPA and befiradol or a pharmaceutically acceptable salt thereof is administered to the patient daily for at least 6 weeks.

The present disclosure further provides a method for treating L-DOPA-induced dyskinesia in a patient in need thereof, comprising daily administering to the patient a therapeutically effective daily dosage of L-DOPA and befiradol or a pharmaceutically acceptable salt thereof, wherein:
a. the method is safe and well tolerated;
b. the therapeutically effective daily dosage of befiradol or a pharmaceutically acceptable salt thereof provides, after at least two weeks of treatment according to the method, a mean maximum befiradol blood plasma concentration of at least about 30 ng/ml; and
c. the therapeutically effective daily dosage of befiradol or a pharmaceutically acceptable salt thereof is at least about 5.7 mmol.

The present disclosure further provides a method for treating L-DOPA-induced dyskinesia in a patient in need thereof, comprising daily administering to the patient a therapeutically effective daily dosage of L-DOPA and befiradol or a pharmaceutically acceptable salt thereof, wherein:
a. the method is safe and well tolerated;
b. the method comprises a titration phase followed by a treatment phase, wherein:
   i. at the beginning of the titration phase the befiradol or a pharmaceutically acceptable salt thereof is administered at an initial daily dosage that is lower than the therapeutically effective daily dosage of befiradol or a pharmaceutically acceptable salt thereof;
   ii. during the titration phase the initial daily dosage of befiradol or a pharmaceutically acceptable salt thereof is gradually increased to the therapeutically effective daily dosage of befiradol or a pharmaceutically acceptable salt thereof; and
   iii. during the treatment phase the therapeutically effective daily dosage of L-DOPA and befiradol or a pharmaceutically acceptable salt thereof is administered daily;
c. the therapeutically effective daily dosage of befiradol or a pharmaceutically acceptable salt thereof provides, after at least two weeks of treatment according to the method, a mean maximum befiradol blood plasma concentration of at least about 30 ng/mL; and
d. the therapeutically effective daily dosage of befiradol or a pharmaceutically acceptable salt thereof is administered in a sustained release pharmaceutical composition.

The present disclosure further provides a method for treating L-DOPA-induced dyskinesia in a patient in need thereof, comprising daily administering to the patient a therapeutically effective daily dosage of L-DOPA and befiradol or a pharmaceutically acceptable salt thereof, wherein:
a. the method is safe and well tolerated;
b. the method comprises a titration phase followed by a treatment phase, wherein:
   i. at the beginning of the titration phase the befiradol or a pharmaceutically acceptable salt thereof is administered at an initial daily dosage that is lower than the therapeutically effective daily dosage of befiradol or a pharmaceutically acceptable salt thereof;
   ii. during the titration phase the initial daily dosage of befiradol or a pharmaceutically acceptable salt thereof is gradually increased to the therapeutically effective daily dosage of befiradol or a pharmaceutically acceptable salt thereof; and
   iii. during the treatment phase the therapeutically effective daily dosage of L-DOPA and befiradol or a pharmaceutically acceptable salt thereof is administered daily;
c. the therapeutically effective daily dosage of befiradol or a pharmaceutically acceptable salt thereof provides, after at least two weeks of treatment according to the method, a mean maximum befiradol blood plasma concentration of at least about 30 ng/ml; and
d. the treatment phase lasts at least 6 weeks.

The present disclosure further provides a method for treating L-DOPA-induced dyskinesia in a patient in need thereof, comprising daily administering to the patient a therapeutically effective daily dosage of L-DOPA and befiradol or a pharmaceutically acceptable salt thereof, wherein:
a. the method is safe and well tolerated;
b. the method comprises a titration phase followed by a treatment phase, wherein:
   i. at the beginning of the titration phase the befiradol or a pharmaceutically acceptable salt thereof is administered at an initial daily dosage that is lower than the therapeutically effective daily dosage of befiradol or a pharmaceutically acceptable salt thereof;
   ii. during the titration phase the initial daily dosage of befiradol or a pharmaceutically acceptable salt thereof is gradually increased to the therapeutically effective daily dosage of befiradol or a pharmaceutically acceptable salt thereof; and
   iii. during the treatment phase the therapeutically effective daily dosage of L-DOPA and befiradol or a pharmaceutically acceptable salt thereof is administered daily;
c. the therapeutically effective daily dosage of befiradol or a pharmaceutically acceptable salt thereof provides, after at least two weeks of treatment according to the method, a mean maximum befiradol blood plasma concentration of at least about 30 ng/ml; and
d. the therapeutically effective daily dosage of befiradol or a pharmaceutically acceptable salt thereof is at least about 5.7 mmol.

In some embodiments of the methods for treating L-DOPA-induced dyskinesia disclosed herein, if parkinsonian symptoms of at least 15 subjects with L-DOPA-induced dyskinesia are evaluated at baseline and after treatment according to the method for at least 14 days in a double blind, placebo-controlled clinical trial, the parkinsonian symptoms of the treated subjects do not become significantly more severe compared to baseline.

In some embodiments of the methods for treating L-DOPA-induced dyskinesia disclosed herein, if parkinsonian symptoms of at least 15 subjects with L-DOPA-induced dyskinesia are evaluated at baseline and after treatment according to the method for at least 14 days in a double blind, placebo-controlled clinical trial, the parkinsonian symptoms of the treated subjects significantly improve compared to baseline.

In some embodiments of the methods for treating L-DOPA-induced dyskinesia disclosed herein, if parkinsonian symptoms are evaluated at baseline and after treatment in a double blind, placebo-controlled clinical trial in which a first group comprising at least 15 subjects with L-DOPA-induced dyskinesia is treated according to the method for at least 14 days and a second group comprising at least 7 subjects with L-DOPA-induced dyskinesia is treated according to the method for the at least 14 days but using placebo instead of befiradol or a pharmaceutically acceptable salt thereof, the parkinsonian symptoms of the first group, but not the second group, significantly improve compared to baseline.

In some embodiments of the methods for treating L-DOPA-induced dyskinesia disclosed herein, if parkinsonian motor symptoms of at least 15 subjects with L-DOPA-induced dyskinesia are evaluated at baseline and after treatment according to the method for at least 14 days in a double blind, placebo-controlled clinical trial, the parkinsonian motor symptoms of the treated subjects do not become significantly more severe compared to baseline.

In some embodiments of the methods for treating L-DOPA-induced dyskinesia disclosed herein, if parkinsonian motor symptoms of at least 15 subjects with L-DOPA-induced dyskinesia are evaluated at baseline and after treatment according to the method for at least 14 days in a double blind, placebo-controlled clinical trial, the parkinsonian motor symptoms of the treated subjects significantly improve compared to baseline.

In some embodiments of the methods for treating L-DOPA-induced dyskinesia disclosed herein, if parkinsonian motor symptoms are evaluated at baseline and after treatment in a double blind, placebo-controlled clinical trial in which a first group comprising at least 15 subjects with L-DOPA-induced dyskinesia is treated according to the method for at least 14 days and a second group comprising at least 7 subjects with L-DOPA-induced dyskinesia is treated according to the method for the at least 14 days but using placebo instead of befiradol or a pharmaceutically acceptable salt thereof, the parkinsonian motor symptoms of the first group, but not the second group, significantly improve compared to baseline.

In some embodiments, the therapeutically effective daily dosage of L-DOPA and befiradol or a pharmaceutically acceptable salt thereof is administered to the patient daily for at least six weeks. In some embodiments, the therapeutically effective daily dosage of L-DOPA and befiradol or a pharmaceutically acceptable salt thereof is administered to the patient daily for at least eight weeks. In some embodiments, the therapeutically effective daily dosage of L-DOPA and befiradol or a pharmaceutically acceptable salt thereof is administered to the patient daily for at least ten weeks.

### B. Parkinson's Disease

The present disclosure provides a method for treating Parkinson's disease in a patient in need thereof, comprising daily administering to the patient a therapeutically effective daily dosage of L-DOPA and befiradol or a pharmaceutically acceptable salt thereof, wherein:
a. the method is safe and well tolerated;
b. the therapeutically effective daily dosage of befiradol or a pharmaceutically acceptable salt thereof provides, after at least two weeks of treatment according to the method, a mean maximum befiradol blood plasma concentration of at least about 30 ng/ml; and
c. the therapeutically effective daily dosage of befiradol or a pharmaceutically acceptable salt thereof is administered in a sustained release pharmaceutical composition.

The present disclosure further provides a method for treating Parkinson's disease in a patient in need thereof, comprising daily administering to the patient a therapeutically effective daily dosage of L-DOPA and befiradol or a pharmaceutically acceptable salt thereof, wherein:
a. the method is safe and well tolerated;
b. the therapeutically effective daily dosage of befiradol or a pharmaceutically acceptable salt thereof provides, after at least two weeks of treatment according to the method, a mean maximum befiradol blood plasma concentration of at least about 30 ng/ml; and
c. the therapeutically effective daily dosage of L-DOPA and befiradol or a pharmaceutically acceptable salt thereof is administered to the patient daily for at least 6 weeks.

The present disclosure further provides a method for treating Parkinson's disease in a patient in need thereof, comprising daily administering to the patient a therapeutically effective daily dosage of L-DOPA and befiradol or a pharmaceutically acceptable salt thereof, wherein:
a. the method is safe and well tolerated;
b. the therapeutically effective daily dosage of befiradol or a pharmaceutically acceptable salt thereof provides, after at least two weeks of treatment according to the method, a mean maximum befiradol blood plasma concentration of at least about 30 ng/mL; and
c. the therapeutically effective daily dosage of befiradol or a pharmaceutically acceptable salt thereof is at least about 5.7 mmol.

The present disclosure further provides a method for treating Parkinson's disease in a patient in need thereof, comprising daily administering to the patient a therapeutically effective daily dosage of L-DOPA and befiradol or a pharmaceutically acceptable salt thereof, wherein:
a. the method is safe and well tolerated;
b. the method comprises a titration phase followed by a treatment phase, wherein:
   i. at the beginning of the titration phase the befiradol or a pharmaceutically acceptable salt thereof is administered at an initial daily dosage that is lower than the therapeutically effective daily dosage of befiradol or a pharmaceutically acceptable salt thereof;
   ii. during the titration phase the initial daily dosage of befiradol or a pharmaceutically acceptable salt thereof is gradually increased to the therapeutically effective daily dosage of befiradol or a pharmaceutically acceptable salt thereof; and
   iii. during the treatment phase the therapeutically effective daily dosage of L-DOPA and befiradol or a pharmaceutically acceptable salt thereof is administered daily;
c. the therapeutically effective daily dosage of befiradol or a pharmaceutically acceptable salt thereof provides, after at least two weeks of treatment according to the method, a mean maximum befiradol blood plasma concentration of at least about 30 ng/ml; and
d. the therapeutically effective daily dosage of befiradol or a pharmaceutically acceptable salt thereof is administered in a sustained release pharmaceutical composition.

The present disclosure further provides a method for treating Parkinson's disease in a patient in need thereof, comprising daily administering to the patient a therapeutically effective daily dosage of L-DOPA and befiradol or a pharmaceutically acceptable salt thereof, wherein:
a. the method is safe and well tolerated;
b. the method comprises a titration phase followed by a treatment phase, wherein:
   i. at the beginning of the titration phase the befiradol or a pharmaceutically acceptable salt thereof is administered at an initial daily dosage that is lower than the therapeutically effective daily dosage of befiradol or a pharmaceutically acceptable salt thereof;
   ii. during the titration phase the initial daily dosage of befiradol or a pharmaceutically acceptable salt thereof is gradually increased to the therapeutically effective daily dosage of befiradol or a pharmaceutically acceptable salt thereof; and
   iii. during the treatment phase the therapeutically effective daily dosage of L-DOPA and befiradol or a pharmaceutically acceptable salt thereof is administered daily;
c. the therapeutically effective daily dosage of befiradol or a pharmaceutically acceptable salt thereof provides, after at least two weeks of treatment according to the method, a mean maximum befiradol blood plasma concentration of at least about 30 ng/mL; and
d. the treatment phase lasts at least 6 weeks.

The present disclosure further provides a method for treating Parkinson's disease in a patient in need thereof, comprising daily administering to the patient a therapeutically effective daily dosage of L-DOPA and befiradol or a pharmaceutically acceptable salt thereof, wherein:
a. the method is safe and well tolerated;
b. the method comprises a titration phase followed by a treatment phase, wherein:
   i. at the beginning of the titration phase the befiradol or a pharmaceutically acceptable salt thereof is administered at an initial daily dosage that is lower than the therapeutically effective daily dosage of befiradol or a pharmaceutically acceptable salt thereof;
   ii. during the titration phase the initial daily dosage of befiradol or a pharmaceutically acceptable salt thereof is gradually increased to the therapeutically effective daily dosage of befiradol or a pharmaceutically acceptable salt thereof; and
   iii. during the treatment phase the therapeutically effective daily dosage of L-DOPA and befiradol or a pharmaceutically acceptable salt thereof is administered daily;
c. the therapeutically effective daily dosage of befiradol or a pharmaceutically acceptable salt thereof provides, after at least two weeks of treatment according to the method, a mean maximum befiradol blood plasma concentration of at least about 30 ng/mL; and
d. the therapeutically effective daily dosage of befiradol or a pharmaceutically acceptable salt thereof is at least about 5.7 mmol.

The present disclosure further provides a method for treating Parkinson's disease in a patient that is being treated with an initial therapeutically effective daily dosage of L-DOPA, comprising the steps of:
a. administering to the patient a therapeutically effective daily dosage of befiradol or a pharmaceutically acceptable salt thereof, and
b. administering to the patient a subsequent therapeutically effective daily dosage of L-DOPA; wherein:
   i. the subsequent therapeutically effective daily dosage of L-DOPA is lower than the initial therapeutically effective daily dosage of L-DOPA,
   ii. the therapeutically effective daily dosage of befiradol or a pharmaceutically acceptable salt thereof provides, after at least two weeks of treatment according to the method, a mean maximum befiradol blood plasma concentration of at least about 30 ng/ml,
   iii. the method is safe and well tolerated, and
   iv. the method does not significantly worsen Parkinson's disease symptoms.
      In some embodiments, the subsequent therapeutically effective daily dosage of L-DOPA is at least 5% lower than the initial therapeutically effective daily dosage of L-DOPA. In some embodiments, the subsequent therapeutically effective daily dosage of L-DOPA is at least 10% lower than the initial therapeutically effective daily dosage of L-DOPA. In some embodiments, the subsequent therapeutically effective daily dosage of L-DOPA is at least 15% lower than the initial therapeutically effective daily dosage of L-DOPA. In some embodiments, the subsequent therapeutically effective daily dosage of L-DOPA is at least 20% lower than the initial therapeutically effective daily dosage of L-DOPA. In some embodiments, the subsequent therapeutically effective daily dosage of L-DOPA is at least 25% lower than the initial therapeutically effective daily dosage of L-DOPA. In some embodiments, the subsequent therapeutically effective daily dosage of L-DOPA is at least 30% lower than the initial therapeutically effective daily dosage of L-DOPA. In some embodiments, the subsequent therapeutically effective daily dosage of L-DOPA is at least 35% lower than the initial therapeutically effective daily dosage of L-DOPA. In some embodiments, the subsequent therapeutically effective daily dosage of L-DOPA is at least 40% lower than the initial therapeutically effective daily dosage of L-DOPA. In some embodiments, the subsequent therapeutically effective daily dosage of L-DOPA is at least 45% lower than the initial therapeutically effective daily dosage of L-DOPA. In some embodiments, the subsequent therapeutically effective daily dosage of L-DOPA is at least 50% lower than the initial therapeutically effective daily dosage of L-DOPA. In some embodiments, the method significantly improves Parkinson's disease symptoms.

The present disclosure further provides a method for treating Parkinson's disease in a patient that is being treated with an initial daily dosage of L-DOPA that is sub-optimal due to L-DOPA-related adverse events, comprising the steps of:
a. administering to the patient a therapeutically effective daily dosage of befiradol or a pharmaceutically acceptable salt thereof, and
b. administering to the patient a subsequent therapeutically effective daily dosage of L-DOPA; wherein:
   i. the subsequent therapeutically effective daily dosage of L-DOPA is the same as or higher than the initial daily dosage of L-DOPA,
   ii. the therapeutically effective daily dosage of befiradol or a pharmaceutically acceptable salt thereof provides, after at least two weeks of treatment according to the method, a mean maximum befiradol blood plasma concentration of at least about 30 ng/ml,
   iii. the method does not significantly increase L-DOPA-related adverse events, and
   iv. the method significantly improves Parkinson's disease symptoms.
      In some embodiments, the subsequent therapeutically effective daily dosage of L-DOPA is the same as the initial daily dosage of L-DOPA. In some embodiments, the subsequent therapeutically effective daily dosage of L-DOPA is at least 5% higher than the initial daily dosage of L-DOPA. In some embodiments, the subsequent therapeutically effective daily dosage of L-DOPA is at least 10% higher than the initial daily dosage of L-DOPA. In some embodiments, the subsequent therapeutically effective daily dosage of L-DOPA is at least 15% higher than the initial daily dosage of L-DOPA. In some embodiments, the subsequent therapeutically effective daily dosage of L-DOPA is at least 20% higher than the initial daily dosage of L-DOPA. In some embodiments, the subsequent therapeutically effective daily dosage of L-DOPA is at least 25% higher than the initial daily dosage of L-DOPA. In some embodiments, the subsequent therapeutically effective daily dosage of L-DOPA is at least 30% higher than the initial daily dosage of L-DOPA. In some embodiments, the subsequent therapeutically effective daily dosage of L-DOPA is at least 35% higher than the initial daily dosage of L-DOPA. In some embodiments, the subsequent therapeutically effective daily dosage of L-DOPA is at least 40% higher than the initial daily dosage of L-DOPA. In some embodiments, the subsequent therapeutically effective daily dosage of L-DOPA is at least 45% higher than the initial daily dosage of L-DOPA. In some embodiments, the subsequent therapeutically effective daily dosage of L-DOPA is at least 50% higher than the initial daily dosage of L-DOPA.

The present disclosure further provides a method for treating Parkinson's disease in a patient that is being treated with an initial daily dosage of L-DOPA that is sub-optimal due to L-DOPA-related adverse events, comprising the steps of:
a. administering to the patient a therapeutically effective daily dosage of befiradol or a pharmaceutically acceptable salt thereof, and
b. administering to the patient a subsequent therapeutically effective daily dosage of L-DOPA; wherein:
   i. the subsequent therapeutically effective daily dosage of L-DOPA is the same as or higher than the initial daily dosage of L-DOPA,
   ii. the therapeutically effective daily dosage of befiradol or a pharmaceutically acceptable salt thereof provides, after at least two weeks of treatment according to the method, a mean maximum befiradol blood plasma concentration of at least about 30 ng/ml,
   iii. the method is safe and well tolerated, and
   iv. the method significantly improves Parkinson's disease symptoms.
      In some embodiments, the subsequent therapeutically effective daily dosage of L-DOPA is the same as the initial daily dosage of L-DOPA. In some embodiments, the subsequent therapeutically effective daily dosage of L-DOPA is at least 5% higher than the initial daily dosage of L-DOPA. In some embodiments, the subsequent therapeutically effective daily dosage of L-DOPA is at least 10% higher than the initial daily dosage of L-DOPA. In some embodiments, the subsequent therapeutically effective daily dosage of L-DOPA is at least 15% higher than the initial daily dosage of L-DOPA. In some embodiments, the subsequent therapeutically effective daily dosage of L-DOPA is at least 20% higher than the initial daily dosage of L-DOPA. In some embodiments, the subsequent therapeutically effective daily dosage of L-DOPA is at least 25% higher than the initial daily dosage of L-DOPA. In some embodiments, the subsequent therapeutically effective daily dosage of L-DOPA is at least 30% higher than the initial daily dosage of L-DOPA. In some embodiments, the subsequent therapeutically effective daily dosage of L-DOPA is at least 35% higher than the initial daily dosage of L-DOPA. In some embodiments, the subsequent therapeutically effective daily dosage of L-DOPA is at least 40% higher than the initial daily dosage of L-DOPA. In some embodiments, the subsequent therapeutically effective daily dosage of L-DOPA is at least 45% higher than the initial daily dosage of L-DOPA. In some embodiments, the subsequent therapeutically effective daily dosage of L-DOPA is at least 50% higher than the initial daily dosage of L-DOPA.

The present disclosure further provides a method for treating Parkinson's disease in a patient that is not being treated with L-DOPA, comprising daily administering to the patient a therapeutically effective daily dosage of L-DOPA and befiradol or a pharmaceutically acceptable salt thereof, wherein the therapeutically effective daily dosage of L-DOPA is lower than it would be if the patient was not treated with befiradol or pharmaceutically acceptable salt thereof. In some embodiments, the therapeutically effective daily dosage of L-DOPA is at least 5% lower than it would be if the patient was not treated with befiradol or a pharmaceutically acceptable salt thereof. In some embodiments, the therapeutically effective daily dosage of L-DOPA is at least 10% lower than it would be if the patient was not treated with befiradol or a pharmaceutically acceptable salt thereof. In some embodiments, the therapeutically effective daily dosage of L-DOPA is at least 15% lower than it would be if the patient was not treated with befiradol or a pharmaceutically acceptable salt thereof. In some embodiments, the therapeutically effective daily dosage of L-DOPA is at least 20% lower than it would be if the patient was not treated with befiradol or a pharmaceutically acceptable salt thereof. In some embodiments, the therapeutically effective daily dosage of L-DOPA is at least 25% lower than it would be if the patient was not treated with befiradol or a pharmaceutically acceptable salt thereof. In some embodiments, the therapeutically effective daily dosage of L-DOPA is at least 30% lower than it would be if the patient was not treated with befiradol or a pharmaceutically acceptable salt thereof. In some embodiments, the therapeutically effective daily dosage of L-DOPA is at least 35% lower than it would be if the patient was not treated with befiradol or a pharmaceutically acceptable salt thereof. In some embodiments, the therapeutically effective daily dosage of L-DOPA is at least 40% lower than it would be if the patient was not treated with befiradol or a pharmaceutically acceptable salt thereof. In some embodiments, the therapeutically effective daily dosage of L-DOPA is at least 45% lower than it would be if the patient was not treated with befiradol or a pharmaceutically acceptable salt thereof. In some embodiments, the therapeutically effective daily dosage of L-DOPA is at least 50% lower than it would be if the patient was not treated with befiradol or a pharmaceutically acceptable salt thereof.

In some embodiments, the therapeutically effective daily dosage of L-DOPA and befiradol or a pharmaceutically acceptable salt thereof is administered to the patient daily for at least six weeks. In some embodiments, the therapeutically effective daily dosage of L-DOPA and befiradol or a pharmaceutically acceptable salt thereof is administered to the patient daily for at least eight weeks. In some embodiments, the therapeutically effective daily dosage of L-DOPA and befiradol or a pharmaceutically acceptable salt thereof is administered to the patient daily for at least ten weeks.

### C. Parkinson's Disease Motor Disability

The present disclosure provides a method for treating Parkinson's disease motor disability in a patient in need thereof, comprising daily administering to the patient a therapeutically effective daily dosage of L-DOPA and befiradol or a pharmaceutically acceptable salt thereof, wherein:
a. the method is safe and well tolerated;
b. the therapeutically effective daily dosage of befiradol or a pharmaceutically acceptable salt thereof provides, after at least two weeks of treatment according to the method, a mean maximum befiradol blood plasma concentration of at least about 30 ng/mL; and
c. the therapeutically effective daily dosage of befiradol or a pharmaceutically acceptable salt thereof is administered in a sustained release pharmaceutical composition.

The present disclosure further provides a method for treating Parkinson's disease motor disability in a patient in need thereof, comprising daily administering to the patient a therapeutically effective daily dosage of L-DOPA and befiradol or a pharmaceutically acceptable salt thereof, wherein:
a. the method is safe and well tolerated;
b. the therapeutically effective daily dosage of befiradol or a pharmaceutically acceptable salt thereof provides, after at least two weeks of treatment according to the method, a mean maximum befiradol blood plasma concentration of at least about 30 ng/ml; and
c. the therapeutically effective daily dosage of L-DOPA and befiradol or a pharmaceutically acceptable salt thereof is administered to the patient daily for at least 6 weeks.

The present disclosure further provides a method for treating Parkinson's disease motor disability in a patient in need thereof, comprising daily administering to the patient a therapeutically effective daily dosage of L-DOPA and befiradol or a pharmaceutically acceptable salt thereof, wherein:
a. the method is safe and well tolerated;
b. the therapeutically effective daily dosage of befiradol or a pharmaceutically acceptable salt thereof provides, after at least two weeks of treatment according to the method, a mean maximum befiradol blood plasma concentration of at least about 30 ng/ml; and
c. the therapeutically effective daily dosage of befiradol or a pharmaceutically acceptable salt thereof is at least about 5.7 mmol.

The present disclosure further provides a method for treating Parkinson's disease motor disability in a patient in need thereof, comprising daily administering to the patient a therapeutically effective daily dosage of L-DOPA and befiradol or a pharmaceutically acceptable salt thereof, wherein:
a. the method is safe and well tolerated;
b. the method comprises a titration phase followed by a treatment phase, wherein:
   i. at the beginning of the titration phase the befiradol or a pharmaceutically acceptable salt thereof is administered at an initial daily dosage that is lower than the therapeutically effective daily dosage of befiradol or a pharmaceutically acceptable salt thereof;
   ii. during the titration phase the initial daily dosage of befiradol or a pharmaceutically acceptable salt thereof is gradually increased to the therapeutically effective daily dosage of befiradol or a pharmaceutically acceptable salt thereof; and
   iii. during the treatment phase the therapeutically effective daily dosage of L-DOPA and befiradol or a pharmaceutically acceptable salt thereof is administered daily;
c. the therapeutically effective daily dosage of befiradol or a pharmaceutically acceptable salt thereof provides, after at least two weeks of treatment according to the method, a mean maximum befiradol blood plasma concentration of at least about 30 ng/ml; and
d. the therapeutically effective daily dosage of befiradol or a pharmaceutically acceptable salt thereof is administered in a sustained release pharmaceutical composition.

The present disclosure further provides a method for treating Parkinson's disease motor disability in a patient in need thereof, comprising daily administering to the patient a therapeutically effective daily dosage of L-DOPA and befiradol or a pharmaceutically acceptable salt thereof, wherein:
a. the method is safe and well tolerated;
b. the method comprises a titration phase followed by a treatment phase, wherein:
   i. at the beginning of the titration phase the befiradol or a pharmaceutically acceptable salt thereof is administered at an initial daily dosage that is lower than the therapeutically effective daily dosage of befiradol or a pharmaceutically acceptable salt thereof;
   ii. during the titration phase the initial daily dosage of befiradol or a pharmaceutically acceptable salt thereof is gradually increased to the therapeutically effective daily dosage of befiradol or a pharmaceutically acceptable salt thereof; and
   iii. during the treatment phase the therapeutically effective daily dosage of L-DOPA and befiradol or a pharmaceutically acceptable salt thereof is administered daily;
c. the therapeutically effective daily dosage of befiradol or a pharmaceutically acceptable salt thereof provides, after at least two weeks of treatment according to the method, a mean maximum befiradol blood plasma concentration of at least about 30 ng/mL; and
d. the treatment phase lasts at least 6 weeks.

The present disclosure further provides a method for treating Parkinson's disease motor disability in a patient in need thereof, comprising daily administering to the patient a therapeutically effective daily dosage of L-DOPA and befiradol or a pharmaceutically acceptable salt thereof, wherein:
a. the method is safe and well tolerated;
b. the method comprises a titration phase followed by a treatment phase, wherein:
   i. at the beginning of the titration phase the befiradol or a pharmaceutically acceptable salt thereof is administered at an initial daily dosage that is lower than the therapeutically effective daily dosage of befiradol or a pharmaceutically acceptable salt thereof;
   ii. during the titration phase the initial daily dosage of befiradol or a pharmaceutically acceptable salt thereof is gradually increased to the therapeutically effective daily dosage of befiradol or a pharmaceutically acceptable salt thereof; and
   iii. during the treatment phase the therapeutically effective daily dosage of L-DOPA and befiradol or a pharmaceutically acceptable salt thereof is administered daily;
c. the therapeutically effective daily dosage of befiradol or a pharmaceutically acceptable salt thereof provides, after at least two weeks of treatment according to the method, a mean maximum befiradol blood plasma concentration of at least about 30 ng/mL; and
d. the therapeutically effective daily dosage of befiradol or a pharmaceutically acceptable salt thereof is at least about 5.7 mmol.

The present disclosure further provides a method for treating Parkinson's disease motor disability in a patient that is being treated with an initial therapeutically effective daily dosage of L-DOPA, comprising the steps of:
a. administering to the patient a therapeutically effective daily dosage of befiradol or a pharmaceutically acceptable salt thereof, and
b. administering to the patient a subsequent therapeutically effective daily dosage of L-DOPA; wherein:
   i. the subsequent therapeutically effective daily dosage of L-DOPA is lower than the initial therapeutically effective daily dosage of L-DOPA,
   ii. the therapeutically effective daily dosage of befiradol or a pharmaceutically acceptable salt thereof provides, after at least two weeks of treatment according to the method, a mean maximum befiradol blood plasma concentration of at least about 30 ng/ml,
   iii. the method is safe and well tolerated, and
   iv. the method does not significantly worsen Parkinson's disease motor symptoms.
      In some embodiments, the subsequent therapeutically effective daily dosage of L-DOPA is at least 5% lower than the initial therapeutically effective daily dosage of L-DOPA. In some embodiments, the subsequent therapeutically effective daily dosage of L-DOPA is at least 10% lower than the initial therapeutically effective daily dosage of L-DOPA. In some embodiments, the subsequent therapeutically effective daily dosage of L-DOPA is at least 15% lower than the initial therapeutically effective daily dosage of L-DOPA. In some embodiments, the subsequent therapeutically effective daily dosage of L-DOPA is at least 20% lower than the initial therapeutically effective daily dosage of L-DOPA. In some embodiments, the subsequent therapeutically effective daily dosage of L-DOPA is at least 25% lower than the initial therapeutically effective daily dosage of L-DOPA. In some embodiments, the subsequent therapeutically effective daily dosage of L-DOPA is at least 30% lower than the initial therapeutically effective daily dosage of L-DOPA. In some embodiments, the subsequent therapeutically effective daily dosage of L-DOPA is at least 35% lower than the initial therapeutically effective daily dosage of L-DOPA. In some embodiments, the subsequent therapeutically effective daily dosage of L-DOPA is at least 40% lower than the initial therapeutically effective daily dosage of L-DOPA. In some embodiments, the subsequent therapeutically effective daily dosage of L-DOPA is at least 45% lower than the initial therapeutically effective daily dosage of L-DOPA. In some embodiments, the subsequent therapeutically effective daily dosage of L-DOPA is at least 50% lower than the initial therapeutically effective daily dosage of L-DOPA. In some embodiments, the method significantly improves Parkinson's disease motor symptoms.

The present disclosure further provides a method for treating Parkinson's disease motor disability in a patient that is being treated with an initial daily dosage of L-DOPA that is sub-optimal due to L-DOPA-related adverse events, comprising the steps of:
a. administering to the patient a therapeutically effective daily dosage of befiradol or a pharmaceutically acceptable salt thereof, and
b. administering to the patient a subsequent therapeutically effective daily dosage of L-DOPA, wherein:
   i. the subsequent therapeutically effective daily dosage of L-DOPA is the same as or higher than the initial daily dosage of L-DOPA,
   ii. the therapeutically effective daily dosage of befiradol or a pharmaceutically acceptable salt thereof provides, after at least two weeks of treatment according to the method, a mean maximum befiradol blood plasma concentration of at least about 30 ng/ml,
   iii. the method does not significantly increase L-DOPA-related adverse events, and
   iv. the method significantly improves Parkinson's disease motor symptoms.
      In some embodiments, the subsequent therapeutically effective daily dosage of L-DOPA is the same as the initial daily dosage of L-DOPA. In some embodiments, the subsequent therapeutically effective daily dosage of L-DOPA is at least 5% higher than the initial daily dosage of L-DOPA. In some embodiments, the subsequent therapeutically effective daily dosage of L-DOPA is at least 10% higher than the initial daily dosage of L-DOPA. In some embodiments, the subsequent therapeutically effective daily dosage of L-DOPA is at least 15% higher than the initial daily dosage of L-DOPA. In some embodiments, the subsequent therapeutically effective daily dosage of L-DOPA is at least 20% higher than the initial daily dosage of L-DOPA. In some embodiments, the subsequent therapeutically effective daily dosage of L-DOPA is at least 25% higher than the initial daily dosage of L-DOPA. In some embodiments, the subsequent therapeutically effective daily dosage of L-DOPA is at least 30% higher than the initial daily dosage of L-DOPA. In some embodiments, the subsequent therapeutically effective daily dosage of L-DOPA is at least 35% higher than the initial daily dosage of L-DOPA. In some embodiments, the subsequent therapeutically effective daily dosage of L-DOPA is at least 40% higher than the initial daily dosage of L-DOPA. In some embodiments, the subsequent therapeutically effective daily dosage of L-DOPA is at least 45% higher than the initial daily dosage of L-DOPA. In some embodiments, the subsequent therapeutically effective daily dosage of L-DOPA is at least 50% higher than the initial daily dosage of L-DOPA.

The present disclosure further provides a method for treating Parkinson's disease motor disability in a patient that is being treated with an initial daily dosage of L-DOPA that is sub-optimal due to L-DOPA-related adverse events, comprising the steps of:
a. administering to the patient a therapeutically effective daily dosage of befiradol or a pharmaceutically acceptable salt thereof, and
b. administering to the patient a subsequent therapeutically effective daily dosage of L-DOPA; wherein:
   i. the subsequent therapeutically effective daily dosage of L-DOPA is the same as or higher than the initial daily dosage of L-DOPA,
   ii. the therapeutically effective daily dosage of befiradol or a pharmaceutically acceptable salt thereof provides, after at least two weeks of treatment according to the method, a mean maximum befiradol blood plasma concentration of at least about 30 ng/ml,
   iii. the method is safe and well tolerated, and
   iv. the method significantly improves Parkinson's disease motor symptoms.
      In some embodiments, the subsequent therapeutically effective daily dosage of L-DOPA is the same as the initial daily dosage of L-DOPA. In some embodiments, the subsequent therapeutically effective daily dosage of L-DOPA is at least 5% higher than the initial daily dosage of L-DOPA. In some embodiments, the subsequent therapeutically effective daily dosage of L-DOPA is at least 10% higher than the initial daily dosage of L-DOPA. In some embodiments, the subsequent therapeutically effective daily dosage of L-DOPA is at least 15% higher than the initial daily dosage of L-DOPA. In some embodiments, the subsequent therapeutically effective daily dosage of L-DOPA is at least 20% higher than the initial daily dosage of L-DOPA. In some embodiments, the subsequent therapeutically effective daily dosage of L-DOPA is at least 25% higher than the initial daily dosage of L-DOPA. In some embodiments, the subsequent therapeutically effective daily dosage of L-DOPA is at least 30% higher than the initial daily dosage of L-DOPA. In some embodiments, the subsequent therapeutically effective daily dosage of L-DOPA is at least 35% higher than the initial daily dosage of L-DOPA. In some embodiments, the subsequent therapeutically effective daily dosage of L-DOPA is at least 40% higher than the initial daily dosage of L-DOPA. In some embodiments, the subsequent therapeutically effective daily dosage of L-DOPA is at least 45% higher than the initial daily dosage of L-DOPA. In some embodiments, the subsequent therapeutically effective daily dosage of L-DOPA is at least 50% higher than the initial daily dosage of L-DOPA.

The present disclosure further provides a method for treating Parkinson's disease motor disability in a patient that is not being treated with L-DOPA, comprising daily administering to the patient a therapeutically effective daily dosage of L-DOPA and befiradol or a pharmaceutically acceptable salt thereof, wherein the therapeutically effective daily dosage of L-DOPA is lower than it would be if the patient was not treated with befiradol or pharmaceutically acceptable salt thereof. In some embodiments, the therapeutically effective daily dosage of L-DOPA is at least 5% lower than it would be if the patient was not treated with befiradol or a pharmaceutically acceptable salt thereof. In some embodiments, the therapeutically effective daily dosage of L-DOPA is at least 10% lower than it would be if the patient was not treated with befiradol or a pharmaceutically acceptable salt thereof. In some embodiments, the therapeutically effective daily dosage of L-DOPA is at least 15% lower than it would be if the patient was not treated with befiradol or a pharmaceutically acceptable salt thereof. In some embodiments, the therapeutically effective daily dosage of L-DOPA is at least 20% lower than it would be if the patient was not treated with befiradol or a pharmaceutically acceptable salt thereof. In some embodiments, the therapeutically effective daily dosage of L-DOPA is at least 25% lower than it would be if the patient was not treated with befiradol or a pharmaceutically acceptable salt thereof. In some embodiments, the therapeutically effective daily dosage of L-DOPA is at least 30% lower than it would be if the patient was not treated with befiradol or a pharmaceutically acceptable salt thereof. In some embodiments, the therapeutically effective daily dosage of L-DOPA is at least 35% lower than it would be if the patient was not treated with befiradol or a pharmaceutically acceptable salt thereof. In some embodiments, the therapeutically effective daily dosage of L-DOPA is at least 40% lower than it would be if the patient was not treated with befiradol or a pharmaceutically acceptable salt thereof. In some embodiments, the therapeutically effective daily dosage of L-DOPA is at least 45% lower than it would be if the patient was not treated with befiradol or a pharmaceutically acceptable salt thereof. In some embodiments, the therapeutically effective daily dosage of L-DOPA is at least 50% lower than it would be if the patient was not treated with befiradol or a pharmaceutically acceptable salt thereof.

In some embodiments, the therapeutically effective daily dosage of L-DOPA and befiradol or a pharmaceutically acceptable salt thereof is administered to the patient daily for at least six weeks. In some embodiments, the therapeutically effective daily dosage of L-DOPA and befiradol or a pharmaceutically acceptable salt thereof is administered to the patient daily for at least eight weeks. In some embodiments, the therapeutically effective daily dosage of L-DOPA and befiradol or a pharmaceutically acceptable salt thereof is administered to the patient daily for at least ten weeks.

### III. Drugs Used in the Therapeutic Methods

### A. L-DOPA

Each method of the present disclosure comprises the administration of a therapeutically effective daily dosage of a) L-DOPA and b) befiradol or a pharmaceutically acceptable salt thereof. The therapeutically effective daily dosage of L-DOPA may be administered using any commercially available pharmaceutical composition of L-DOPA, such as tablets (Sinemet®), extended-release capsules (Rytary®), extended-release tablets (Sinemet CR®) or enteral suspension (Duopa®) comprising a combination of carbidopa and levodopa, or tablets comprising a combination of carbidopa, entacapone, and levodopa (Stalevo®). Guidance concerning a therapeutically effective daily dosage of a L-DOPA pharmaceutical composition can be obtained from government agencies responsible for regulating drugs for human use, such as the FDA (www.fda.gov) and EMA (ema.europa.eu). For example, the approved labeling for Sinemet® tablets, which includes dosage and administration information, is available online at https://www.accessdata.fda.gov/drugsatfda_docs/label/2020/017555s0721bl.pdf.

### B. Befiradol or a Pharmaceutically Acceptable Salt Thereof

### 1. Pharmaceutical Composition

In each method of the present disclosure, the therapeutically effective daily dosage of befiradol or a pharmaceutically acceptable salt thereof is administered in a pharmaceutical composition. Accordingly, provided herein is a pharmaceutical composition comprising befiradol or a pharmaceutically acceptable salt thereof and a pharmaceutically acceptable excipient. Any suitable pharmaceutical composition may be used. In some embodiments, the pharmaceutical composition comprises befiradol and a pharmaceutically acceptable excipient. In some embodiments, the pharmaceutical composition comprises a pharmaceutically acceptable salt of befiradol and a pharmaceutically acceptable excipient. In some embodiments, the pharmaceutical composition comprises befiradol fumarate and a pharmaceutically acceptable excipient.

The daily dosage of befiradol or a pharmaceutically acceptable salt thereof can be administered by any suitable route. In some embodiments, the daily dosage of befiradol or a pharmaceutically acceptable salt thereof is administered transdermally. In some embodiments, the daily dosage of befiradol or a pharmaceutically acceptable salt thereof is administered transdermally from a patch. Preferably, the daily dosage of befiradol or a pharmaceutically acceptable salt thereof is administered orally. More preferably, the daily dosage of befiradol or a pharmaceutically acceptable salt thereof is administered orally as a tablet or capsule.

The daily dosage of befiradol or a pharmaceutically acceptable salt thereof can be administered to the patient each day in one or more doses. In some embodiments, each of the one or more doses of befiradol or a pharmaceutically acceptable salt thereof is administered as a conventional immediate release tablet or capsule. In some embodiments, each of the one or more doses of befiradol or a pharmaceutically acceptable salt thereof is administered as a sustained release tablet or capsule.

Proper formulation is dependent upon the route of administration chosen. For example, a patch may be used for transdermal administration, whereas a tablet, capsule, solution or suspension may be used for oral administration. Any of the well-known techniques and excipients may be used as suitable and as understood in the art (see, e.g., Remington: The Science and Practice of Pharmacy, 20th ed., Gennaro et al. Eds., Lippincott Williams and Wilkins, 2000).

Pharmaceutically acceptable excipients include pharmaceutically acceptable binding agents, lubricants, wetting agents, disintegrants, and the like. Tablets, pills, capsules, troches and the like can contain any of the following excipients, or compounds of a similar nature: a binder such as microcrystalline cellulose, gum tragacanth or gelatin; a diluent such as starch or lactose, a dispersing agent such as alginic acid, sodium starch glycolate, or corn starch; a lubricant such as magnesium stearate; a glidant such as colloidal silicon dioxide. The amount of active ingredient that is combined with the pharmaceutically acceptable excipient(s) to produce a single dosage form will vary depending upon the particular mode of administration and the identity of the dosage form.

In some embodiments, the pharmaceutical composition of befiradol or a pharmaceutically acceptable salt thereof is a sustained release composition. In some embodiments, the sustained release pharmaceutical composition of befiradol or a pharmaceutically acceptable salt thereof is suitable for transdermal administration. In some embodiments, the sustained release pharmaceutical composition of befiradol or a pharmaceutically acceptable salt thereof is suitable for oral administration. In some embodiments, the sustained release pharmaceutical composition of befiradol or a pharmaceutically acceptable salt thereof is suitable for once daily oral dosing. Sustained release pharmaceutical compositions of befiradol or a pharmaceutically acceptable salt thereof suitable for use according to the present methods are described in WO 2016/005527.

To prepare a sustained release pharmaceutical composition of befiradol or a pharmaceutically acceptable salt thereof, it is necessary to control the release of befiradol or a pharmaceutically acceptable salt thereof from the pharmaceutical composition. The release of befiradol or a pharmaceutically acceptable salt thereof from the pharmaceutical composition may be controlled using any suitable method. Systems that can be used to control the release of befiradol are known or will be apparent to those skilled in the art; for example, see WO 2016/005527; Remington, The Science and Practice of Pharmacy (21st Edition, Mack Publishing Company, 2006; Wise, Handbook of Pharmaceutical Controlled Release (Marcel Dekker Publishing Company, 2000)). As examples, mention may be made of the following systems:
Diffusion systems: Characterized by the release rate of drug, being dependent on its diffusion through an inert membrane barrier, usually an insoluble polymer.
Dissolution systems: The release of drug is limited by the rate of dissolution of the system. Sustained-release compositions are made by decreasing their rate of dissolution. The approaches to achieve this decrease include preparing appropriate salts or derivatives, coating the drug with a slowly dissolving material, or incorporating it into a tablet with a slowly dissolving carrier.
Osmotic system: Osmotic pressure is used to generate a constant release of drug, using a semi-permeable membrane that is permeable to water, but not to the drug.
Ion-exchange systems: They generally use resins composed of water-insoluble crosslinked polymers. These polymers contain salt-forming functional groups in repeating positions on the polymer chain. The drug is bound to the resin and released by exchanging with appropriately charged ions in contact with the ion-exchange groups. The free drug diffuses out of the resin. The drug-resin complex is prepared either by repeated exposure of the resin to the drug in a chromatography column, or by prolonged contact in solution.
Swelling and expansion systems: Based on hydrogels which swell fast upon contact with water which leads to a large increase in size and a prolonged transit time in the stomach.
Floating systems: If the dosage form has a lower density than the gastric fluids, it will float on a top of the stomach content, allowing for an increased time span to release the drug.
Bioadhesive or mucoadhesive systems: Based on bioadhesive or mucoadhesive polymers such as polyacrylic acid and chitosan to achieve the dosage forms sticking on to the mucosa.
Matrix systems: Consisting in the direct compression of blends of drug, retardant materials and additives to form a tablet in which drug is embedded in matrix core of the retardant.

In some embodiments, the pharmaceutical composition comprises at least one pharmaceutically acceptable excipient that controls the release of befiradol. Said excipient can be any suitable agent, such as a controlling agent, polymeric agent or coating agent (e.g., ethyl cellulose). Suitable release controlling agents include, for example, cellulose and cellulose derivative, wax, carbomer, polyalkylene polyol, polycarbophil, methacrylic acid derivative, gelatin, gum, polyethylene oxide, and polyvinyl pyrrolidone, or mixtures thereof.

In some embodiments, said excipient is a polymer, either biodegradable or non-biodegradable; preferentially selected from the group consisting of ethyl cellulose, methyl cellulose, hydroxypropyl cellulose, hydroxyethyl cellulose, hydroxymethyl cellulose, hydroxypropyl methyl cellulose, hydroxypropylmethyl cellulose acetate succinate, hydroxypropylmethyl cellulose phthalate, methylcellulose, cellulose acetate, cellulose acetate phthalate, cellulose acetate trimellitate and carboxymethylcellulose sodium; acrylic acid polymers and copolymers (preferably formed from acrylic acid, methacrylic acid, methyl acrylate, ethyl acrylate, methyl methacrylate and/or ethyl methacrylate), and other methacrylic resins that are commercially available under the trade name Eudragit™ (Evonik), including Eudragit™ L30D-55 and LI00-55, Eudragit™, and Eudragit™ NE, RL and RS; vinyl polymers and copolymers such as polyvinyl pyrrolidone, polyvinyl chloride, vinyl acetate, vinyl acetate phthalate, vinyl acetate crotonic acid copolymer, and ethylene-vinyl acetate copolymer; enzymatically degradable polymers such as azo polymers, pectins, chitosan, amylase, carnauba wax, agar-agar, carob gum, shellac gum, gum Arabic, gum tragacanth, guar gum, alginates, xanthan gum, carregeenan, polysaccharides of mannose and galactose, modified starches, and guar gum; polyethylene; zein and shellac, or mixtures thereof. In some preferred embodiments, said polymer is ethyl cellulose.

The pharmaceutical composition can comprise any suitable amount of said excipient that controls the release of befiradol (e.g., ethyl cellulose). In some embodiments, the pharmaceutical composition comprises about 1% to about 30% by weight of the said controlled release excipient with respect to the total weight of the composition, such as about 1% to about 25%, about 1% to about 20%, about 2% to about 20%, about 4% to about 20%, about 4% to about 15%, or about 8% to about 15%.

In some embodiments, the pharmaceutical composition also comprises a plasticizer, for example triglycerides, oleic acid, or a mixture thereof. Other suitable plasticizers include, for example, triethyl citrate, polyethylene glycol, propylene glycol, triacetin, dimethyl phthalate, diethyl phthalate, dibutyl phthalate, dibutyl sebacate, tributyl citrate, triethyl acetyl citrate, glycerolmonostearate, castor oil, acetylated monoglycerides, or a mixture thereof. The pharmaceutical composition can comprise any suitable amount of the plasticizer (e.g., mixture of oleic acid and triglycerides). In some embodiments, the pharmaceutical composition comprises about 0.1% to 15% by weight of the plasticizer, with respect to the total weight of the composition, such as about 0.1% to about 5%, or about 1% to about 5%.

Aqueous dispersions of ethyl cellulose, also comprising a plasticizer, are commercially available under the trade name Surelease™ (Colorcon). The formula of Surelease E-7-19040™ consists in purified water, ethyl cellulose 20 mPa·s, ammonium hydroxide 28%, triglycerides medium-chain and oleic acid.

In some embodiments, the pharmaceutical composition comprises an aqueous dispersion of ethyl cellulose, preferentially Surelease E-7-19040™. The pharmaceutical composition can comprise any suitable amount of Surelease E-7-19040™. In some embodiments, the pharmaceutical composition comprises about 1% to about 30% by dry weight of Surelease E-7-19040™, with respect to the total weight of the composition; such as about 2% to about 25%, about 5% to about 20%, about 7% to about 20%, about 7% to about 15%, about 9% to about 15%, or about 9.88% or about 14.49%.

In some embodiments, the pharmaceutical composition comprises about 1% to about 5% by weight of befiradol or a pharmaceutically acceptable salt thereof; about 75% to about 90% by weight of an inert substrate or filler; about 3% to about 6% by weight of a binder; about 2% to about 20% by weight of an excipient that controls the release, such as about 5% to about 20%, about 5% to about 15%, or about 9% to about 15%; about 1% to about 5% by weight of a plasticizer; and about 0.1% to about 1% by weight of an antiadherent or lubricant, with respect to the total weight of the composition.

### 2. Daily Dosage

In each method of the present disclosure, the befiradol or a pharmaceutically acceptable salt thereof is administered to the patient daily, at a daily dosage that may vary during treatment. For example, the therapeutically effective daily dosage of befiradol or a pharmaceutically acceptable salt thereof may be increased to improve efficacy or decreased to improve tolerability. Also, when beginning treatment according to a method of the present disclosure, the daily dosage of befiradol or a pharmaceutically acceptable salt thereof may be initiated at an amount lower than the therapeutically effective daily dosage of befiradol or a pharmaceutically acceptable salt thereof and gradually increased over time (i.e., titrated) to a therapeutically effective daily dosage.

In some embodiments, the method of the present disclosure comprises a titration phase followed by a treatment phase, wherein:
a. at the beginning of the titration phase the befiradol or a pharmaceutically acceptable salt thereof is administered at an initial daily dosage that is lower than the therapeutically effective daily dosage of befiradol or a pharmaceutically acceptable salt thereof;
b. during the titration phase the initial daily dosage of befiradol or a pharmaceutically acceptable salt thereof is gradually increased to the therapeutically effective daily dosage of befiradol or a pharmaceutically acceptable salt thereof; and
c. during the treatment phase the therapeutically effective daily dosage of L-DOPA and befiradol or a pharmaceutically acceptable salt thereof is administered daily.
   The daily dosage of befiradol or a pharmaceutically acceptable salt thereof at the beginning of the titration phase may be any suitable amount. In some embodiments, the titration phase is initiated at a daily dosage of about 0.63 mmol of befiradol or a pharmaceutically acceptable salt thereof (i.e., about 0.25 mg befiradol free base, about 0.32 mg befiradol fumarate, etc.). In some embodiments, the titration phase is initiated at a daily dosage of about 1.3 mmol of befiradol or a pharmaceutically acceptable salt thereof (i.e., about 0.5 mg befiradol free base, about 0.65 mg befiradol fumarate, etc.). In some embodiments, the titration phase is initiated at a daily dosage of about 1.9 mmol of befiradol or a pharmaceutically acceptable salt thereof (i.e., about 0.75 mg befiradol free base, about 0.97 mg befiradol fumarate, etc.). In some embodiments, the titration phase is initiated at a daily dosage of about 2.5 mmol of befiradol or a pharmaceutically acceptable salt thereof (i.e., about 1 mg befiradol free base, about 1.3 mg befiradol fumarate, etc.).

In some embodiments, the titration phase is at least one week. In some embodiments, the titration phase is at least two weeks. In some embodiments, the titration phase is at least three weeks. In some embodiments, the titration phase is at least four weeks. In some embodiments, the titration phase is about two weeks. In some embodiments, the titration phase is about three weeks. In some embodiments, the titration phase is about four weeks. In some embodiments, the titration phase is about five weeks. In some embodiments, the titration phase is about six weeks. In some embodiments, the titration phase is about seven weeks. In some embodiments, the titration phase is about eight weeks.

In some embodiments, the initial daily dosage at the beginning of the titration phase is maintained for about one to five days, and then about every one to five days thereafter the daily dosage of befiradol or a pharmaceutically acceptable salt thereof is increased by about 0.3 mmol to about 1.3 mmol until the therapeutically effective daily dosage is reached. In some embodiments, the initial daily dosage at the beginning of the titration phase is maintained for about three to five days, and then about every three to five days thereafter the daily dosage of befiradol or a pharmaceutically acceptable salt thereof is increased by about 0.3 mmol to about 1.3 mmol until the therapeutically effective daily dosage is reached. In some embodiments, the initial daily dosage at the beginning of the titration phase is maintained for about four days, and then about every four days thereafter the daily dosage of befiradol or a pharmaceutically acceptable salt thereof is increased by about 0.63 mmol until the therapeutically effective daily dosage is reached.

In some embodiments, the initial daily dosage at the beginning of the titration phase is about 0.3 mmol to about 2.5 mmol of befiradol or a pharmaceutically acceptable salt thereof. In some embodiments, the initial daily dosage at the beginning of the titration phase is about 0.3 mmol to about 1.9 mmol of befiradol or a pharmaceutically acceptable salt thereof. In some embodiments, the initial daily dosage at the beginning of the titration phase is about 0.3 mmol to about 1.3 mmol of befiradol or a pharmaceutically acceptable salt thereof. In some embodiments, the initial daily dosage at the beginning of the titration phase is about 0.63 mmol of befiradol or a pharmaceutically acceptable salt thereof. In some embodiments, the initial daily dosage at the beginning of the titration phase is about 1.3 mmol of befiradol or a pharmaceutically acceptable salt thereof. In some embodiments, the initial daily dosage at the beginning of the titration phase is about 1.9 mmol of befiradol or a pharmaceutically acceptable salt thereof.

In some embodiments, the initial daily dosage of befiradol or a pharmaceutically acceptable salt thereof at the beginning of the titration phase is at least 90% lower than the therapeutically effective daily dosage of befiradol or a pharmaceutically acceptable salt thereof. In some embodiments, the initial daily dosage at the beginning of the titration phase is at least 80% lower than the therapeutically effective daily dosage. In some embodiments, the initial daily dosage at the beginning of the titration phase is at least 70% lower than the therapeutically effective daily dosage. In some embodiments, the initial daily dosage at the beginning of the titration phase is at least 60% lower than the therapeutically effective daily dosage. In some embodiments, the initial daily dosage at the beginning of the titration phase is at least 50% lower than the therapeutically effective daily dosage. In some embodiments, the initial daily dosage at the beginning of the titration phase is at least 40% lower than the therapeutically effective daily dosage. In some embodiments, the initial daily dosage at the beginning of the titration phase is at least 30% lower than the therapeutically effective daily dosage.

In some embodiments, the therapeutically effective daily dosage of befiradol or a pharmaceutically acceptable salt thereof is about 2.5 mmol to about 12.7 mmol. In some embodiments, the therapeutically effective daily dosage of befiradol or a pharmaceutically acceptable salt thereof is about 2.5 mmol to about 10.2 mmol. In some embodiments, the therapeutically effective daily dosage of befiradol or a pharmaceutically acceptable salt thereof is about 5.1 mmol to about 10.2 mmol. In some embodiments, the therapeutically effective daily dosage of befiradol or a pharmaceutically acceptable salt thereof is about 2.5 mmol to about 7.6 mmol. In some embodiments, the therapeutically effective daily dosage of befiradol or a pharmaceutically acceptable salt thereof is about 5.1 mmol to about 7.6 mmol. In some embodiments, the therapeutically effective daily dosage of befiradol or a pharmaceutically acceptable salt thereof is about 2.5 mmol. In some embodiments, the therapeutically effective daily dosage of befiradol or a pharmaceutically acceptable salt thereof is about 3.8 mmol. In some embodiments, the therapeutically effective daily dosage of befiradol or a pharmaceutically acceptable salt thereof is about 5.1 mmol. In some embodiments, the therapeutically effective daily dosage of befiradol or a pharmaceutically acceptable salt thereof is about 6.3 mmol. In some embodiments, the therapeutically effective daily dosage of befiradol or a pharmaceutically acceptable salt thereof is about 7.6 mmol. In some embodiments, the therapeutically effective daily dosage of befiradol or a pharmaceutically acceptable salt thereof is about 8.9 mmol. In some embodiments, the therapeutically effective daily dosage of befiradol or a pharmaceutically acceptable salt thereof is about 10.2 mmol. In some embodiments, the therapeutically effective daily dosage of befiradol or a pharmaceutically acceptable salt thereof is about 11.4 mmol. In some embodiments, the therapeutically effective daily dosage of befiradol or a pharmaceutically acceptable salt thereof is a daily dosage of about 12.7 mmol. In some embodiments, the therapeutically effective daily dosage of befiradol or a pharmaceutically acceptable salt thereof is about 1.3 mg to about 6.5 mg befiradol fumarate. In some embodiments, the therapeutically effective daily dosage of befiradol or a pharmaceutically acceptable salt thereof is about 1.3 mg to about 5.2 mg befiradol fumarate. In some embodiments, the therapeutically effective daily dosage of befiradol or a pharmaceutically acceptable salt thereof is about 2.6 mg to about 5.2 mg befiradol fumarate. In some embodiments, the therapeutically effective daily dosage of befiradol or a pharmaceutically acceptable salt thereof is about 1.3 mg to about 3.9 mg befiradol fumarate. In some embodiments, the therapeutically effective daily dosage of befiradol or a pharmaceutically acceptable salt thereof is about 2.6 mg to about 3.9 mg befiradol fumarate. In some embodiments, the therapeutically effective daily dosage of befiradol or a pharmaceutically acceptable salt thereof is about 1.3 mg befiradol fumarate. In some embodiments, the therapeutically effective daily dosage of befiradol or a pharmaceutically acceptable salt thereof is about 1.9 mg befiradol fumarate. In some embodiments, the therapeutically effective daily dosage of befiradol or a pharmaceutically acceptable salt thereof is about 2.6 mg befiradol fumarate. In some embodiments, the therapeutically effective daily dosage of befiradol or a pharmaceutically acceptable salt thereof is about 3.2 mg befiradol fumarate. In some embodiments, the therapeutically effective daily dosage of befiradol or a pharmaceutically acceptable salt thereof is about 3.9 mg befiradol fumarate. In some embodiments, the therapeutically effective daily dosage of befiradol or a pharmaceutically acceptable salt thereof is about 4.5 mg befiradol fumarate. In some embodiments, the therapeutically effective daily dosage of befiradol or a pharmaceutically acceptable salt thereof is about 5.2 mg befiradol fumarate. In some embodiments, the therapeutically effective daily dosage of befiradol or a pharmaceutically acceptable salt thereof is about 5.8 mg befiradol fumarate. In some embodiments, the therapeutically effective daily dosage of befiradol or a pharmaceutically acceptable salt thereof is about 6.5 mg befiradol fumarate.

In some embodiments, the therapeutically effective daily dosage of befiradol or a pharmaceutically acceptable salt thereof that is used in the methods of the present disclosure is at least about 5.7 mmol (i.e., at least about 2.25 mg befiradol free base, at least about 2.9 mg befiradol fumarate, etc.) (note: befiradol free base has a molecular weight of about 394 g/mol and the pharmaceutically acceptable salt befiradol fumarate has a molecular weight of about 510 g/mol). In some embodiments, the therapeutically effective daily dosage of befiradol or a pharmaceutically acceptable salt thereof is at least about 6.3 mmol. In some embodiments, the therapeutically effective daily dosage of befiradol or a pharmaceutically acceptable salt thereof is at least about 7 mmol. In some embodiments, the therapeutically effective daily dosage of befiradol or a pharmaceutically acceptable salt thereof is at least about 7.6 mmol. In some embodiments, the therapeutically effective daily dosage of befiradol or a pharmaceutically acceptable salt thereof is about 5.7 mmol to about 12.7 mmol. In some embodiments, the therapeutically effective daily dosage of befiradol or a pharmaceutically acceptable salt thereof is about 6.3 mmol to about 12.7 mmol. In some embodiments, the therapeutically effective daily dosage of befiradol or a pharmaceutically acceptable salt thereof is about 7 mmol mg to about 12.7 mmol. In some embodiments, the therapeutically effective daily dosage of befiradol or a pharmaceutically acceptable salt thereof is about 7.6 mmol to about 12.7 mmol. In some embodiments, the therapeutically effective daily dosage of befiradol or a pharmaceutically acceptable salt thereof is about 5.7 mmol to about 10.2 mmol. In some embodiments, the therapeutically effective daily dosage of befiradol or a pharmaceutically acceptable salt thereof is about 6.3 mmol to about 10.2 mmol. In some embodiments, the therapeutically effective daily dosage of befiradol or a pharmaceutically acceptable salt thereof is about 7 mmol mg to about 10.2 mmol. In some embodiments, the therapeutically effective daily dosage of befiradol or a pharmaceutically acceptable salt thereof is about 7.6 mmol to about 10.2 mmol.

In each method of the present disclosure, the daily dosage of befiradol or a pharmaceutically acceptable salt thereof-whether the therapeutically effective daily dosage or any lower daily dosage administered during the titration phase, if applicable—may, unless otherwise specified, be administered to the patient each day in any suitable number of doses. In some embodiments, the daily dosage of befiradol or a pharmaceutically acceptable salt thereof is administered to the patient each day in multiple doses. In some embodiments, the daily dosage of befiradol or a pharmaceutically acceptable salt thereof is administered to the patient each day in two to four, preferably equal, doses. In some embodiments, the daily dosage of befiradol or a pharmaceutically acceptable salt thereof is administered to the patient each day in two, preferably equal, doses (e.g., b.i.d.). For example, if the daily dosage of befiradol or a pharmaceutically acceptable salt thereof is about 5 mmol (about 2 mg befiradol free base), the daily dosage may be administered each day as a morning dose of about 2.5 mmol (about 1 mg befiradol free base) and an evening dose of about 2.5 mmol (1 mg befiradol free base).

In some embodiments, the daily dosage of befiradol or a pharmaceutically acceptable salt thereof is administered to the patient each day in multiple doses, and at least one of the daily doses is administered as a sustained release pharmaceutical composition of befiradol or a pharmaceutically acceptable salt thereof. In some embodiments, the daily dosage of befiradol or a pharmaceutically acceptable salt thereof is administered to the patient each day in multiple doses, and each dose is administered as a sustained release pharmaceutical composition of befiradol or a pharmaceutically acceptable salt thereof. In some embodiments, the daily dosage of befiradol or a pharmaceutically acceptable salt thereof is administered to the patient each day transdermally as a sustained release patch pharmaceutical composition of befiradol or a pharmaceutically acceptable salt thereof. Such a sustained release patch pharmaceutical composition may contain an amount of befiradol or a pharmaceutically acceptable salt thereof sufficient for one day, wherein the patch is replaced each day, or it may contain an amount of befiradol or a pharmaceutically acceptable salt thereof sufficient for more than one day. For example, such a sustained release patch pharmaceutical composition may contain an amount of befiradol or a pharmaceutically acceptable salt thereof sufficient for one week, wherein the patch administers a therapeutically effective daily dosage of befiradol or a pharmaceutically acceptable salt thereof each day for seven days and is then replaced at the end of each week.

In some embodiments, the daily dosage of befiradol or a pharmaceutically acceptable salt thereof is administered to the patient each day as a single daily oral dose in a sustained release pharmaceutical composition. In some embodiments, the daily dosage of befiradol or a pharmaceutically acceptable salt thereof is administered to the patient each day in the morning as a single daily oral dose in a sustained release pharmaceutical composition. In some embodiments, the daily dosage of befiradol or a pharmaceutically acceptable salt thereof is administered to the patient each day in the evening as a single daily oral dose in a sustained release pharmaceutical composition. In some embodiments, the daily dosage of befiradol or a pharmaceutically acceptable salt thereof is administered to the patient each day as two daily oral doses in a sustained release pharmaceutical composition. In some embodiments, the daily dosage of befiradol or a pharmaceutically acceptable salt thereof is administered to the patient each day as two daily oral doses—one in the morning and one in the evening—in a sustained release pharmaceutical composition.

### 3. Plasma Concentration

In each method of the present disclosure, the therapeutically effective daily dosage of befiradol or a pharmaceutically acceptable salt thereof provides, after at least two weeks of treatment according to the method, a mean maximum befiradol blood plasma concentration of at least about 30 ng/mL. In some embodiments, the therapeutically effective daily dosage of befiradol or a pharmaceutically acceptable salt thereof provides, after at least two weeks of treatment according to the method, a mean maximum befiradol blood plasma concentration of at least about 40 ng/ml. In some embodiments, the therapeutically effective daily dosage of befiradol or a pharmaceutically acceptable salt thereof provides, after at least two weeks of treatment according to the method, a mean maximum befiradol blood plasma concentration of at least about 50 ng/mL. In some embodiments, the therapeutically effective daily dosage of befiradol or a pharmaceutically acceptable salt thereof provides, after at least two weeks of treatment according to the method, a mean maximum befiradol blood plasma concentration of at least about 60 ng/mL. In some embodiments, the therapeutically effective daily dosage of befiradol or a pharmaceutically acceptable salt thereof provides, after at least two weeks of treatment according to the method, a mean maximum befiradol blood plasma concentration of at least about 70 ng/mL. In some embodiments, the therapeutically effective daily dosage of befiradol or a pharmaceutically acceptable salt thereof provides, after at least two weeks of treatment according to the method, a mean maximum befiradol blood plasma concentration of about 30 ng/ml to about 120 ng/mL. In some embodiments, the therapeutically effective daily dosage of befiradol or a pharmaceutically acceptable salt thereof provides, after at least two weeks of treatment according to the method, a mean maximum befiradol blood plasma concentration of about 30 ng/ml to about 100 ng/mL. In some embodiments, the therapeutically effective daily dosage of befiradol or a pharmaceutically acceptable salt thereof provides, after at least two weeks of treatment according to the method, a mean maximum befiradol blood plasma concentration of about 40 ng/mL to about 100 ng/mL. In some embodiments, the therapeutically effective daily dosage of befiradol or a pharmaceutically acceptable salt thereof provides, after at least two weeks of treatment according to the method, a mean maximum befiradol blood plasma concentration of about 50 ng/mL to about 90 ng/mL. In some embodiments, the therapeutically effective daily dosage of befiradol or a pharmaceutically acceptable salt thereof provides, after at least two weeks of treatment according to the method, a mean maximum befiradol blood plasma concentration of about 60 ng/mL to about 80 ng/mL.

The mean maximum befiradol blood plasma concentration after at least two weeks of treatment according to the method with a therapeutically effective daily dosage of befiradol or a pharmaceutically acceptable salt thereof may be determined in a suitable clinical trial in which subjects representative of the Parkinson's disease patients treated by the methods of the present disclosure are administered a therapeutically effective daily dosage of befiradol or a pharmaceutically acceptable salt thereof in accordance with a method of the present disclosure for at least two weeks. The clinical trial subjects preferably have Parkinson's disease (optionally with L-DOPA-induced dyskinesia) and are concurrently treated with a therapeutically effective daily dosage of L-DOPA, but for the sake of convenience the subjects may be healthy volunteers with ages representative of patients with Parkinson's disease, which tends to begin in middle or late life, usually age 60 or older. By way of example, the clinical trial subjects may include at least 6 male subjects and at least 6 female subjects, at least 50 years old if they do not have Parkinson's disease, who are administered a therapeutically effective daily dosage of befiradol or a pharmaceutically acceptable salt thereof in accordance with a method of the present disclosure for a period of at least 14 days. Such a clinical trial may be performed analogously to Example 1, below. Note that in Example 1, the befiradol blood plasma concentrations were measured only once on each of study days 14, 25, 28, 42, and 70 (i.e., Visits 4, 5, 6, 7, and 9, respectively) and the length of time after administering the morning befiradol dose was not strictly observed. Therefore, the mean maximum blood plasma concentration following the morning befiradol dose on the tested days may be higher than the befiradol blood plasma concentrations reported in Table 5, below. Such a clinical trial may also be performed analogously to Example 4, below, using subjects with Parkinson's disease or subjects at least 50 years old. Such a clinical trial may also be performed analogously to Example 5, below (elderly subjects). It is believed that clinical trials performed using subjects with Parkinson's disease, healthy subjects at least 50 years old, or healthy elderly subjects will yield similar mean maximum befiradol blood plasma concentrations. However, if there is a significant difference in mean maximum befiradol blood plasma concentrations in healthy subjects at least 50 years old and/or healthy elderly subjects compared to subjects with Parkinson's disease, then the clinical trial should not be performed with healthy subjects and should instead be performed with subjects with Parkinson's disease. It is believed that administering befiradol with or without food will not significantly affect the mean maximum befiradol blood plasma concentration. However, if there is a significant food effect, then each dose of befiradol or a pharmaceutically acceptable salt thereof should be administered with food (e.g., as in Example 1, below). In some embodiments, if the therapeutically effective daily dosage of befiradol or a pharmaceutically acceptable salt thereof is administered for a period of at least 14 days to at least 6 males and at least 6 females with Parkinson's disease in such a clinical trial, it provides on or after day 14 of such period a mean maximum plasma concentration of befiradol greater than about 30 ng/mL. In some embodiments, if the therapeutically effective daily dosage of befiradol or a pharmaceutically acceptable salt thereof is administered for a period of at least 14 days to at least 6 males and at least 6 females with Parkinson's disease in such a clinical trial, it provides on or after day 14 of such period a mean maximum plasma concentration of befiradol greater than about 40 ng/mL. In some embodiments, if the therapeutically effective daily dosage of befiradol or a pharmaceutically acceptable salt thereof is administered for a period of at least 14 days to at least 6 males and at least 6 females with Parkinson's disease in such a clinical trial, it provides on or after day 14 of such period a mean maximum plasma concentration of befiradol greater than about 50 ng/ml. In some embodiments, if the therapeutically effective daily dosage of befiradol or a pharmaceutically acceptable salt thereof is administered for a period of at least 14 days to at least 6 males and at least 6 females with Parkinson's disease in such a clinical trial, it provides on or after day 14 of such period a mean maximum plasma concentration of befiradol greater than about 60 ng/mL. In some embodiments, if the therapeutically effective daily dosage of befiradol or a pharmaceutically acceptable salt thereof is administered for a period of at least 14 days to at least 6 males and at least 6 females with Parkinson's disease in such a clinical trial, it provides on or after day 14 of such period a mean maximum plasma concentration of befiradol greater than about 70 ng/mL. In some embodiments, if the therapeutically effective daily dosage of befiradol or a pharmaceutically acceptable salt thereof is administered for a period of at least 14 days to at least 6 males and at least 6 females with Parkinson's disease in such a clinical trial, it provides on or after day 14 of such period a mean maximum plasma concentration of befiradol greater than about 80 ng/mL. In some embodiments, if the therapeutically effective daily dosage of befiradol or a pharmaceutically acceptable salt thereof is administered for a period of at least 14 days to at least 6 males and at least 6 females with Parkinson's disease in such a clinical trial, it provides on or after day 14 of such period a mean maximum plasma concentration of befiradol greater than about 90 ng/mL. In some embodiments, if the therapeutically effective daily dosage of befiradol or a pharmaceutically acceptable salt thereof is administered for a period of at least 14 days to at least 6 males and at least 6 females with Parkinson's disease in such a clinical trial, it provides on or after day 14 of such period a mean maximum plasma concentration of befiradol greater than about 100 ng/mL. In some embodiments, if the therapeutically effective daily dosage of befiradol or a pharmaceutically acceptable salt thereof is administered for a period of at least 14 days to at least 6 males and at least 6 females aged 50 or older in such a clinical trial, it provides on or after day 14 of such period a mean maximum plasma concentration of befiradol greater than about 30 ng/mL. In some embodiments, if the therapeutically effective daily dosage of befiradol or a pharmaceutically acceptable salt thereof is administered for a period of at least 14 days to at least 6 males and at least 6 females aged 50 or older in such a clinical trial, it provides on or after day 14 of such period a mean maximum plasma concentration of befiradol greater than about 40 ng/mL. In some embodiments, if the therapeutically effective daily dosage of befiradol or a pharmaceutically acceptable salt thereof is administered for a period of at least 14 days to at least 6 males and at least 6 females aged 50 or older in such a clinical trial, it provides on or after day 14 of such period a mean maximum plasma concentration of befiradol greater than about 50 ng/mL. In some embodiments, if the therapeutically effective daily dosage of befiradol or a pharmaceutically acceptable salt thereof is administered for a period of at least 14 days to at least 6 males and at least 6 females aged 50 or older in such a clinical trial, it provides on or after day 14 of such period a mean maximum plasma concentration of befiradol greater than about 60 ng/mL. In some embodiments, if the therapeutically effective daily dosage of befiradol or a pharmaceutically acceptable salt thereof is administered for a period of at least 14 days to at least 6 males and at least 6 females aged 50 or older in such a clinical trial, it provides on or after day 14 of such period a mean maximum plasma concentration of befiradol greater than about 70 ng/mL. In some embodiments, if the therapeutically effective daily dosage of befiradol or a pharmaceutically acceptable salt thereof is administered for a period of at least 14 days to at least 6 males and at least 6 females aged 50 or older in such a clinical trial, it provides on or after day 14 of such period a mean maximum plasma concentration of befiradol greater than about 80 ng/ml. In some embodiments, if the therapeutically effective daily dosage of befiradol or a pharmaceutically acceptable salt thereof is administered for a period of at least 14 days to at least 6 males and at least 6 females aged 50 or older in such a clinical trial, it provides on or after day 14 of such period a mean maximum plasma concentration of befiradol greater than about 90 ng/mL. In some embodiments, if the therapeutically effective daily dosage of befiradol or a pharmaceutically acceptable salt thereof is administered for a period of at least 14 days to at least 6 males and at least 6 females aged 50 or older in such a clinical trial, it provides on or after day 14 of such period a mean maximum plasma concentration of befiradol greater than about 100 ng/mL.

In some embodiments, the therapeutically effective daily dosage of befiradol or a pharmaceutically acceptable salt thereof is administered each day orally in one or more doses, and each such dose, if administered once to at least 6 males and at least 6 females in a clinical trial without a befiradol daily dosage up-titration period, provides a mean maximum plasma concentration of befiradol greater than about 15 ng/mL. In some embodiments, each such dose provides a mean maximum plasma concentration of befiradol greater than about 20 ng/mL in such a clinical trial. In some embodiments, each such dose provides a mean maximum plasma concentration of befiradol greater than about 21 ng/mL in such a clinical trial. In some embodiments, each such dose provides a mean maximum plasma concentration of befiradol greater than about 22 ng/mL in such a clinical trial. In some embodiments, each such dose provides a mean maximum plasma concentration of befiradol greater than about 23 ng/mL in such a clinical trial. In some embodiments, each such dose provides a mean maximum plasma concentration of befiradol greater than about 24 ng/mL in such a clinical trial. In some embodiments, each such dose provides a mean maximum plasma concentration of befiradol greater than about 25 ng/mL in such a clinical trial. In some embodiments, each such dose provides a mean maximum plasma concentration of befiradol greater than about 26 ng/mL in such a clinical trial. In some embodiments, each such dose provides a mean maximum plasma concentration of befiradol greater than about 27 ng/ml in such a clinical trial. In some embodiments, each such dose provides a mean maximum plasma concentration of befiradol greater than about 28 ng/mL in such a clinical trial. In some embodiments, each such dose provides a mean maximum plasma concentration of befiradol greater than about 29 ng/mL in such a clinical trial. In some embodiments, each such dose provides a mean maximum plasma concentration of befiradol greater than about 30 ng/mL in such a clinical trial. In some embodiments, each such dose provides a mean maximum plasma concentration of befiradol greater than about 31 ng/mL in such a clinical trial. In some embodiments, each such dose provides a mean maximum plasma concentration of befiradol greater than about 32 ng/mL in such a clinical trial. In some embodiments, each such dose provides a mean maximum plasma concentration of befiradol greater than about 33 ng/mL in such a clinical trial. In some embodiments, each such dose provides a mean maximum plasma concentration of befiradol greater than about 34 ng/mL in such a clinical trial. In some embodiments, each such dose provides a mean maximum plasma concentration of befiradol greater than about 35 ng/ml in such a clinical trial. Since the mean maximum plasma concentration of befiradol may be higher in elderly subjects (see Example 2, below), it is preferred that the 6 males and 6 females in such a clinical trial are elderly. In some embodiments, such a clinical trial is performed analogously to Example 3, below. If befiradol exhibits a significant food effect, then the dose of befiradol or a pharmaceutically acceptable salt thereof should be administered with food (e.g., as in Example 1, below).

### IV. Safety and Tolerability

Each method of the present disclosure is safe and well tolerated. The safety and tolerability of a method of the present disclosure may be shown in any suitable manner. For example, the safety and tolerability of a method of the present disclosure may be shown in a clinical trial in which a group of subjects is administered, in accordance with a method of the present disclosure, in blinded fashion, a therapeutically effective daily dosage of befiradol or a pharmaceutically acceptable salt thereof (subgroup 1) or a placebo (subgroup 2), and the adverse events experienced by the two subgroups during the clinical trial compared (e.g., proportion of subjects in each subgroup that experience at least one adverse event; the number of adverse events per subject in each subgroup; the incidence of a particular adverse event, such as dizziness, in each subgroup; adverse event severity; etc.). Each subgroup may comprise at least 9 subjects. The therapeutically effective daily dosage of befiradol or a pharmaceutically acceptable salt thereof or placebo may be administered to the respective subgroups for at least two weeks. The clinical trial may be performed analogously to Example 1, below. In some embodiments, the safety profile of subgroup 1 and subgroup 2 is similar. In some embodiments, the adverse events experienced by subgroup 1 and subgroup 2 is similar. In some embodiments, the proportion of subjects that experience at least one adverse event in subgroup 1 and subgroup 2 is similar. In some embodiments, the number of adverse events per subject in subgroup 1 and subgroup 2 is similar. In some embodiments, the severity of adverse events in subgroup 1 and subgroup 2 is similar. In some embodiments, the incidence of a particular adverse event, such as dizziness, in subgroup 1 and subgroup 2 is similar. In some embodiments, the incidence of dizziness in subgroup 1 and subgroup 2 is similar. In some embodiments, the incidence of headache in subgroup 1 and subgroup 2 is similar. In some embodiments, the safety profile of subgroup 1 and subgroup 2 is not significantly different. In some embodiments, the adverse events experienced by subgroup 1 and subgroup 2 is not significantly different. In some embodiments, the proportion of subjects that experience at least one adverse event in subgroup 1 and subgroup 2 is not significantly different. In some embodiments, the number of adverse events per subject in subgroup 1 and subgroup 2 is not significantly different. In some embodiments, the severity of adverse events in subgroup 1 and subgroup 2 is not significantly different. In some embodiments, the incidence of a particular adverse event, such as dizziness, in subgroup 1 and subgroup 2 is not significantly different. In some embodiments, the incidence of dizziness in subgroup 1 and subgroup 2 is not significantly different. In some embodiments, the incidence of headache in subgroup 1 and subgroup 2 is not significantly different.

### V. Efficacy

The efficacy of treating L-DOPA-induced dyskinesia may be determined by evaluating symptom severity using any suitable dyskinesia severity rating scale. In some embodiments, the efficacy of treating L-DOPA-induced dyskinesia is determined by evaluating symptom severity using the Unified Dyskinesia Rating Scale (UDysRS) total score (Parts 1-4) or objective score (Parts 3 and 4). Using the UDysRS Parts 1˜4 or Parts 3 and 4, the signs and symptoms of L-DOPA-induced dyskinesia can be scored in multiple patients before and after treatment according to a method of the present disclosure and the results compared, e.g., as described in Example 1, below.

The efficacy of treating Parkinson's disease, or of treating parkinsonian symptoms, may be determined by evaluating symptom severity using any suitable Parkinson's disease severity rating scale. In some embodiments, the efficacy of treating Parkinson's disease, or of treating parkinsonian symptoms, is determined by evaluating symptom severity using the Unified Parkinson's Disease Rating Scale (UPDRS) total score (Parts 1-4). Using the UPDRS Parts 1-4, the signs and symptoms of Parkinson's disease can be scored in multiple patients before and after treatment according to a method of the present disclosure and the results compared, e.g., as described in Example 1, below.

The efficacy of treating Parkinson's disease motor disability, or of treating parkinsonian motor symptoms, may be determined by evaluating symptom severity using any suitable Parkinson's disease motor disability severity rating scale. In some embodiments, the efficacy of treating Parkinson's disease motor disability, or of treating parkinsonian motor symptoms, is determined by evaluating symptom severity using the Unified Parkinson's Disease Rating Scale (UPDRS) Part 3 score. Using the UPDRS Part 3, the signs and symptoms of Parkinson's disease motor disability can be scored in multiple patients before and after treatment according to a method of the present disclosure and the results compared, e.g., as described in Example 1, below.

In some embodiments, the method of the present disclosure does not significantly worsen Parkinson's disease symptoms, as measured by the UPDRS. In some embodiments, the method does not significantly worsen Parkinson's disease motor symptoms, as measured by UPDRS Part 3. In some embodiments, the method of the present disclosure significantly improves Parkinson's disease symptoms, as measured by the UPDRS. In some embodiments, the method significantly improves Parkinson's disease motor symptoms, as measured by UPDRS Part 3. In some embodiments, the method significantly improves L-DOPA-induced dyskinesia (LID), as measured by the UDysRS. In some embodiments, the method significantly improves Parkinson's disease symptoms, as indicated by a significant reduction in UPDRS total score. In some embodiments, the method significantly improves Parkinson's disease motor symptoms, as indicated by a significant reduction in UPDRS total score for Part 3. In some embodiments, the method significantly improves L-DOPA-induced dyskinesia (LID), as indicated by a significant reduction in UDysRS total score. In some embodiments, the method significantly improves L-DOPA-induced dyskinesia (LID), as indicated by a significant reduction in UDysRS total score for Parts 3 and 4. In some embodiments, the method significantly improves L-DOPA-induced dyskinesia (LID), as indicated by a significant reduction in UDysRS total score or a significant reduction in UDysRS total score for Parts 3 and 4. In some embodiments, the method significantly improves L-DOPA-induced dyskinesia (LID), as indicated by a significant reduction in UDysRS total score and a significant reduction in UDysRS total score for Parts 3 and 4.

In some embodiments, the UPDRS total score is used to assess the parkinsonian symptoms of the patients, wherein a significant reduction in UPDRS total score indicates a significant improvement in parkinsonian symptoms. In some embodiments, UPDRS Part 3 is used to assess the parkinsonian motor symptoms of the patients, wherein a significant reduction in UPDRS Part 3 score indicates a significant improvement in parkinsonian motor symptoms. In some embodiments, the method significantly improves parkinsonian symptoms. In some embodiments, the method does not significantly worsen parkinsonian symptoms. In some embodiments, the method significantly improves parkinsonian motor symptoms. In some embodiments, the method does not significantly worsen parkinsonian motor symptoms. In some embodiments, the method significantly improves anti-parkinsonian efficacy. In some embodiments, the method does not significantly worsen anti-parkinsonian efficacy. In some embodiments, the method significantly improves anti-parkinsonian efficacy as measured by UPDRS total score. In some embodiments, the method does not significantly worsen anti-parkinsonian efficacy as measured by UPDRS total score. In some embodiments, the method significantly improves anti-parkinsonian efficacy as measured by UPDRS Part 3. In some embodiments, the method does not significantly worsen anti-parkinsonian efficacy as measured by UPDRS Part 3.

### VI. Enumerated Embodiments

Also provided are the following enumerated embodiments of the methods of the present disclosure:

Embodiment 1: A method of treating Parkinson's disease in a patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the method is safe and well tolerated, and
c. the once daily oral dose of befiradol or a pharmaceutically acceptable salt thereof, if administered once to at least 6 elderly male subjects and at least 6 elderly female subjects in a clinical trial without a befiradol dose up-titration period, provides a mean maximum plasma concentration of befiradol greater than about 15 ng/mL.

Embodiment 2. A method of treating Parkinson's disease motor disability in a patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the method is safe and well tolerated, and
c. the once daily oral dose of befiradol or a pharmaceutically acceptable salt thereof, if administered once to at least 6 elderly male subjects and at least 6 elderly female subjects in a clinical trial without a befiradol dose up-titration period, provides a mean maximum plasma concentration of befiradol greater than about 15 ng/mL.

Embodiment 3. A method of treating L-DOPA-induced dyskinesia in a patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the method is safe and well tolerated, and
c. the once daily oral dose of befiradol or a pharmaceutically acceptable salt thereof, if administered once to at least 6 elderly male subjects and at least 6 elderly female subjects in a clinical trial without a befiradol dose up-titration period, provides a mean maximum plasma concentration of befiradol greater than about 15 ng/mL.

Embodiment 4. A method of treating L-DOPA-induced dyskinesia in a patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the once daily oral dose of befiradol or a pharmaceutically acceptable salt thereof, if administered once to at least 6 elderly male subjects and at least 6 elderly female subjects in a clinical trial without a befiradol dose up-titration period, provides a mean maximum plasma concentration of befiradol greater than about 15 ng/ml, and
c. if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the parkinsonian symptoms of the first group significantly improve after the at least 14 days.

Embodiment 4a. A method of treating L-DOPA-induced dyskinesia in a human patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the once daily oral dose of befiradol or a pharmaceutically acceptable salt thereof, when administered once to at least 6 elderly males and at least 6 elderly females in a clinical trial without a befiradol dose up-titration period, provides a mean maximum plasma concentration of befiradol greater than about 15 ng/ml, and
c. if parkinsonian symptoms of at least 15 subjects with Parkinson's disease and L-DOPA-induced dyskinesia are evaluated at baseline and after treatment according to the method for at least 14 days in a double blind, placebo-controlled clinical trial, the parkinsonian symptoms of the treated subjects significantly improve compared to baseline.

Embodiment 4b. A method of treating L-DOPA-induced dyskinesia in a human patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the once daily oral dose of befiradol or a pharmaceutically acceptable salt thereof, when administered once to at least 6 elderly males and at least 6 elderly females in a clinical trial without a befiradol dose up-titration period, provides a mean maximum plasma concentration of befiradol greater than about 15 ng/ml, and
c. if parkinsonian symptoms of at least 15 subjects with Parkinson's disease and L-DOPA-induced dyskinesia are evaluated at baseline and after treatment according to the method for at least 14 days in a double blind, placebo-controlled clinical trial, the parkinsonian symptoms of the treated subjects do not become significantly more severe compared to baseline.

Embodiment 5. A method of treating L-DOPA-induced dyskinesia in a patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the once daily oral dose of befiradol or a pharmaceutically acceptable salt thereof, if administered once to at least 6 elderly male subjects and at least 6 elderly female subjects in a clinical trial without a befiradol dose up-titration period, provides a mean maximum plasma concentration of befiradol greater than about 15 ng/mL, and
c. if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the parkinsonian motor symptoms of the first group significantly improve after the at least 14 days.

Embodiment 5a. A method of treating L-DOPA-induced dyskinesia in a human patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the once daily oral dose of befiradol or a pharmaceutically acceptable salt thereof, when administered once to at least 6 elderly males and at least 6 elderly females in a clinical trial without a befiradol dose up-titration period, provides a mean maximum plasma concentration of befiradol greater than about 15 ng/ml, and
c. if parkinsonian motor symptoms of at least 15 subjects with Parkinson's disease and L-DOPA-induced dyskinesia are evaluated at baseline and after treatment according to the method for at least 14 days in a double blind, placebo-controlled clinical trial, the parkinsonian motor symptoms of the treated subjects significantly improve compared to baseline.

Embodiment 5b. A method of treating L-DOPA-induced dyskinesia in a human patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the once daily oral dose of befiradol or a pharmaceutically acceptable salt thereof, when administered once to at least 6 elderly males and at least 6 elderly females in a clinical trial without a befiradol dose up-titration period, provides a mean maximum plasma concentration of befiradol greater than about 15 ng/ml, and
c. if parkinsonian motor symptoms of at least 15 subjects with Parkinson's disease and L-DOPA-induced dyskinesia are evaluated at baseline and after treatment according to the method for at least 14 days in a double blind, placebo-controlled clinical trial, the parkinsonian motor symptoms of the treated subjects do not become significantly more severe compared to baseline.

Embodiment 6. A method of treating Parkinson's disease in a patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the method is safe and well tolerated, and
c. the once daily oral dose of befiradol or a pharmaceutically acceptable salt thereof, if administered daily for a period of at least 14 days to at least 6 elderly male subjects and at least 6 elderly female subjects in a clinical trial, provides on the last day of such period a mean maximum plasma concentration of befiradol greater than about 30 ng/mL.

Embodiment 6a. A method of treating Parkinson's disease in a human patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the method is safe and well tolerated, and
c. the once daily oral dose of befiradol or a pharmaceutically acceptable salt thereof, when administered daily for a period of at least 14 days to at least 6 elderly males and at least 6 elderly females in a clinical trial, provides on or after day 14 within such period a mean maximum plasma concentration of befiradol greater than about 30 ng/mL.

Embodiment 7. A method of treating Parkinson's disease motor disability in a patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the method is safe and well tolerated, and
c. the once daily oral dose of befiradol or a pharmaceutically acceptable salt thereof, if administered daily for a period of at least 14 days to at least 6 elderly male subjects and at least 6 elderly female subjects in a clinical trial, provides on the last day of such period a mean maximum plasma concentration of befiradol greater than about 30 ng/mL.

Embodiment 7a. A method of treating Parkinson's disease motor disability in a human patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the method is safe and well tolerated, and
c. the once daily oral dose of befiradol or a pharmaceutically acceptable salt thereof, when administered daily for a period of at least 14 days to at least 6 elderly males and at least 6 elderly females in a clinical trial, provides on or after day 14 within such period a mean maximum plasma concentration of befiradol greater than about 30 ng/mL.

Embodiment 8. A method of treating L-DOPA-induced dyskinesia in a patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the method is safe and well tolerated, and
c. the once daily oral dose of befiradol or a pharmaceutically acceptable salt thereof, if administered daily for a period of at least 14 days to at least 6 elderly male subjects and at least 6 elderly female subjects in a clinical trial, provides on the last day of such period a mean maximum plasma concentration of befiradol greater than about 30 ng/mL.

Embodiment 8a. A method of treating L-DOPA-induced dyskinesia in a human patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the method is safe and well tolerated, and
c. the once daily oral dose of befiradol or a pharmaceutically acceptable salt thereof, when administered daily for a period of at least 14 days to at least 6 elderly males and at least 6 elderly females in a clinical trial, provides on or after day 14 within such period a mean maximum plasma concentration of befiradol greater than about 30 ng/mL.

Embodiment 9. A method of treating L-DOPA-induced dyskinesia in a patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the once daily oral dose of befiradol or a pharmaceutically acceptable salt thereof, if administered daily for a period of at least 14 days to at least 6 elderly male subjects and at least 6 elderly female subjects in a clinical trial, provides on the last day of such period a mean maximum plasma concentration of befiradol greater than about 30 ng/ml, and
c. if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the parkinsonian symptoms of the first group significantly improve after the at least 14 days.

Embodiment 9a. A method of treating L-DOPA-induced dyskinesia in a human patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the once daily oral dose of befiradol or a pharmaceutically acceptable salt thereof, when administered daily for a period of at least 14 days to at least 6 elderly males and at least 6 elderly females in a clinical trial, provides on or after day 14 within such period a mean maximum plasma concentration of befiradol greater than about 30 ng/ml, and
c. if parkinsonian symptoms of at least 15 subjects with Parkinson's disease and L-DOPA-induced dyskinesia are evaluated at baseline and after treatment according to the method for at least 14 days in a double blind, placebo-controlled clinical trial, the parkinsonian symptoms of the treated subjects significantly improve compared to baseline.

Embodiment 9b. A method of treating L-DOPA-induced dyskinesia in a human patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the once daily oral dose of befiradol or a pharmaceutically acceptable salt thereof, when administered daily for a period of at least 14 days to at least 6 elderly males and at least 6 elderly females in a clinical trial, provides on or after day 14 within such period a mean maximum plasma concentration of befiradol greater than about 30 ng/mL, and
c. if parkinsonian symptoms of at least 15 subjects with Parkinson's disease and L-DOPA-induced dyskinesia are evaluated at baseline and after treatment according to the method for at least 14 days in a double blind, placebo-controlled clinical trial, the parkinsonian symptoms of the treated subjects do not become significantly more severe compared to baseline.

Embodiment 9c. A method of treating L-DOPA-induced dyskinesia in a human patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the once daily oral dose of befiradol or a pharmaceutically acceptable salt thereof, when administered once to at least 6 males and at least 6 females in a clinical trial without a befiradol dose up-titration period, provides a mean maximum plasma concentration of befiradol greater than about 15 ng/ml, and
c. if parkinsonian symptoms are evaluated at baseline and after treatment in a double blind, placebo-controlled clinical trial in which a first group comprising at least 15 subjects with Parkinson's disease and L-DOPA-induced dyskinesia is treated according to the method for at least 14 days and a second group comprising at least 7 subjects with Parkinson's disease and L-DOPA-induced dyskinesia is treated according to the method for at least 14 days but using placebo instead of befiradol or a pharmaceutically acceptable salt thereof, the parkinsonian symptoms of the first group, but not the second group, significantly improve compared to baseline.

Embodiment 10. A method of treating L-DOPA-induced dyskinesia in a patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the once daily oral dose of befiradol or a pharmaceutically acceptable salt thereof, if administered daily for a period of at least 14 days to at least 6 elderly male subjects and at least 6 elderly female subjects in a clinical trial, provides on the last day of such period a mean maximum plasma concentration of befiradol greater than about 30 ng/ml, and
c. if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the parkinsonian motor symptoms of the first group significantly improve after the at least 14 days.

Embodiment 10a. A method of treating L-DOPA-induced dyskinesia in a human patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the once daily oral dose of befiradol or a pharmaceutically acceptable salt thereof, when administered daily for a period of at least 14 days to at least 6 elderly males and at least 6 elderly females in a clinical trial, provides on or after day 14 within such period a mean maximum plasma concentration of befiradol greater than about 30 ng/ml, and
c. if parkinsonian motor symptoms of at least 15 subjects with Parkinson's disease and L-DOPA-induced dyskinesia are evaluated at baseline and after treatment according to the method for at least 14 days in a double blind, placebo-controlled clinical trial, the parkinsonian motor symptoms of the treated subjects significantly improve compared to baseline.

Embodiment 10b. A method of treating L-DOPA-induced dyskinesia in a human patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the once daily oral dose of befiradol or a pharmaceutically acceptable salt thereof, when administered daily for a period of at least 14 days to at least 6 elderly males and at least 6 elderly females in a clinical trial, provides on or after day 14 within such period a mean maximum plasma concentration of befiradol greater than about 30 ng/ml, and
c. if parkinsonian motor symptoms of at least 15 subjects with Parkinson's disease and L-DOPA-induced dyskinesia are evaluated at baseline and after treatment according to the method for at least 14 days in a double blind, placebo-controlled clinical trial, the parkinsonian motor symptoms of the treated subjects do not become significantly more severe compared to baseline.

Embodiment 10c. A method of treating L-DOPA-induced dyskinesia in a human patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the once daily oral dose of befiradol or a pharmaceutically acceptable salt thereof, when administered once to at least 6 males and at least 6 females in a clinical trial without a befiradol dose up-titration period, provides a mean maximum plasma concentration of befiradol greater than about 15 ng/ml, and
c. if parkinsonian motor symptoms are evaluated at baseline and after treatment in a double blind, placebo-controlled clinical trial in which a first group comprising at least 15 subjects with Parkinson's disease and L-DOPA-induced dyskinesia is treated according to the method for at least 14 days and a second group comprising at least 7 subjects with Parkinson's disease and L-DOPA-induced dyskinesia is treated according to the method for at least 14 days but using placebo instead of befiradol or a pharmaceutically acceptable salt thereof, the parkinsonian motor symptoms of the first group, but not the second group, significantly improve compared to baseline.

Embodiment 11. A method of treating L-DOPA-induced dyskinesia in a patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose, and
b. if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the parkinsonian symptoms of the first group significantly improve after the at least 14 days.

Embodiment 11a. A method for treating L-DOPA-induced dyskinesia in a human patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose, and
b. if parkinsonian symptoms of at least 15 subjects with Parkinson's disease and L-DOPA-induced dyskinesia are evaluated at baseline and after treatment according to the method for at least 14 days in a double blind, placebo-controlled clinical trial, the parkinsonian symptoms of the treated subjects significantly improve compared to baseline.

Embodiment 12. A method of treating L-DOPA-induced dyskinesia in a patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose, and
b. if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the parkinsonian motor symptoms of the first group significantly improve after the at least 14 days.

Embodiment 12a. A method for treating L-DOPA-induced dyskinesia in a human patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose, and
b. if parkinsonian motor symptoms of at least 15 subjects with Parkinson's disease and L-DOPA-induced dyskinesia are evaluated at baseline and after treatment according to the method for at least 14 days in a double blind, placebo-controlled clinical trial, the parkinsonian motor symptoms of the treated subjects significantly improve compared to baseline.

Embodiment 13. A method of treating Parkinson's disease in a patient that is being stably and optimally treated with a first daily dosage of L-DOPA, comprising the steps of:
a. administering to the patient a therapeutically effective amount of befiradol or a pharmaceutically acceptable salt thereof, and
b. administering a second daily dosage of L-DOPA, wherein the second daily dosage of L-DOPA is lower than the first daily dosage of L-DOPA.

Embodiment 13a. A method of treating Parkinson's disease in a human patient that is being treated with an initial therapeutically effective daily dosage of L-DOPA, comprising the steps of:
a. administering to the patient a therapeutically effective amount of befiradol or a pharmaceutically acceptable salt thereof, and
b. administering to the patient a subsequent therapeutically effective daily dosage of L-DOPA, wherein the subsequent therapeutically effective daily dosage of L-DOPA is lower than the initial therapeutically effective daily dosage of L-DOPA.

Embodiment 14. A method of treating Parkinson's disease motor disability in a patient that is being stably and optimally treated with a first daily dosage of L-DOPA, comprising the steps of:
a. administering to the patient a therapeutically effective amount of befiradol or a pharmaceutically acceptable salt thereof, and
b. administering a second daily dosage of L-DOPA, wherein the second daily dosage of L-DOPA is lower than the first daily dosage of L-DOPA.

Embodiment 14a. A method of treating Parkinson's disease motor disability in a human patient that is being treated with an initial therapeutically effective daily dosage of L-DOPA, comprising the steps of:
a. administering to the patient a therapeutically effective amount of befiradol or a pharmaceutically acceptable salt thereof, and
b. administering to the patient a subsequent therapeutically effective daily dosage of L-DOPA, wherein the subsequent therapeutically effective daily dosage of L-DOPA is lower than the initial therapeutically effective daily dosage of L-DOPA.

Embodiment 15. A method of treating Parkinson's disease in a patient that is being treated with a first daily dosage of L-DOPA that is sub-optimal due to L-DOPA-related adverse events, comprising the steps of:
a. administering to the patient a therapeutically effective amount of befiradol or a pharmaceutically acceptable salt thereof, and
b. administering a second daily dosage of L-DOPA,
   wherein the second daily dosage of L-DOPA is higher than the first daily dosage of L-DOPA, and the method does not significantly increase L-DOPA-related adverse events.

Embodiment 15a. A method of treating Parkinson's disease in a human patient that is being treated with an initial daily dosage of L-DOPA that is sub-optimal due to L-DOPA-related adverse events, comprising the steps of:
a. administering to the patient a therapeutically effective amount of befiradol or a pharmaceutically acceptable salt thereof, and
b. administering to the patient a subsequent therapeutically effective daily dosage of L-DOPA,
   wherein the subsequent therapeutically effective daily dosage of L-DOPA is higher than the initial daily dosage of L-DOPA, and the method does not significantly increase the L-DOPA-related adverse events.

Embodiment 16. A method of treating Parkinson's disease motor disability in a patient that is being treated with a first daily dosage of L-DOPA that is sub-optimal due to L-DOPA-related adverse events, comprising the steps of:
a. administering to the patient a therapeutically effective amount of befiradol or a pharmaceutically acceptable salt thereof, and
b. administering a second daily dosage of L-DOPA,
   wherein the second daily dosage of L-DOPA is higher than the first daily dosage of L-DOPA, and the method does not significantly increase L-DOPA-related adverse events.

Embodiment 16a. A method of treating Parkinson's disease motor disability in a human patient that is being treated with an initial daily dosage of L-DOPA that is sub-optimal due to L-DOPA-related adverse events, comprising the steps of:
a. administering to the patient a therapeutically effective amount of befiradol or a pharmaceutically acceptable salt thereof, and
b. administering to the patient a subsequent therapeutically effective daily dosage of L-DOPA,
   wherein the subsequent therapeutically effective daily dosage of L-DOPA is higher than the initial daily dosage of L-DOPA, and the method does not significantly increase the L-DOPA-related adverse events.

Embodiment 17. A method of treating Parkinson's disease in a patient that is not being treated with L-DOPA, comprising administering to the patient a therapeutically effective amount of L-DOPA and befiradol or a pharmaceutically acceptable salt thereof, wherein the therapeutically effective amount of L-DOPA is lower than it would be if the patient was not treated with befiradol or pharmaceutically acceptable salt thereof.

Embodiment 18. A method of treating Parkinson's disease motor disability in a patient that is not being treated with L-DOPA, comprising administering to the patient a therapeutically effective amount of L-DOPA and befiradol or a pharmaceutically acceptable salt thereof, wherein the therapeutically effective amount of L-DOPA is lower than it would be if the patient was not treated with befiradol or pharmaceutically acceptable salt thereof.

Embodiment 19. The method of any one of Embodiments 1-5, wherein the once daily oral dose of befiradol or a pharmaceutically acceptable salt thereof, if administered once to at least 6 elderly male subjects and at least 6 elderly female subjects in a clinical trial without a befiradol dose up-titration period, provides a mean maximum plasma concentration of befiradol greater than about 20 ng/mL.

Embodiment 20. The method of any one of Embodiments 1-5, 4a, 4b, 5a, or 5b, wherein the once daily oral dose of befiradol or a pharmaceutically acceptable salt thereof, if administered once to at least 6 elderly male subjects and at least 6 elderly female subjects in a clinical trial without a befiradol dose up-titration period, provides a mean maximum plasma concentration of befiradol greater than about 21 ng/mL.

Embodiment 21. The method of any one of Embodiments 1-5, 4a, 4b, 5a, or 5b, wherein the once daily oral dose of befiradol or a pharmaceutically acceptable salt thereof, if administered once to at least 6 elderly male subjects and at least 6 elderly female subjects in a clinical trial without a befiradol dose up-titration period, provides a mean maximum plasma concentration of befiradol greater than about 22 ng/mL.

Embodiment 22. The method of any one of Embodiments 1-5, 4a, 4b, 5a, or 5b, wherein the once daily oral dose of befiradol or a pharmaceutically acceptable salt thereof, if administered once to at least 6 elderly male subjects and at least 6 elderly female subjects in a clinical trial without a befiradol dose up-titration period, provides a mean maximum plasma concentration of befiradol greater than about 23 ng/mL.

Embodiment 23. The method of any one of Embodiments 1-5, 4a, 4b, 5a, or 5b, wherein the once daily oral dose of befiradol or a pharmaceutically acceptable salt thereof, if administered once to at least 6 elderly male subjects and at least 6 elderly female subjects in a clinical trial without a befiradol dose up-titration period, provides a mean maximum plasma concentration of befiradol greater than about 24 ng/mL.

Embodiment 24. The method of any one of Embodiments 1-5, 4a, 4b, 5a, or 5b, wherein the once daily oral dose of befiradol or a pharmaceutically acceptable salt thereof, if administered once to at least 6 elderly male subjects and at least 6 elderly female subjects in a clinical trial without a befiradol dose up-titration period, provides a mean maximum plasma concentration of befiradol greater than about 25 ng/mL.

Embodiment 25. The method of any one of Embodiments 1-5, 4a, 4b, 5a, or 5b, wherein the once daily oral dose of befiradol or a pharmaceutically acceptable salt thereof, if administered once to at least 6 elderly male subjects and at least 6 elderly female subjects in a clinical trial without a befiradol dose up-titration period, provides a mean maximum plasma concentration of befiradol greater than about 30 ng/mL.

Embodiment 26. The method of any one of Embodiments 6-10, 6a, 7a, 8a, 9a-9c, or 10a-10c, wherein the once daily oral dose of befiradol or a pharmaceutically acceptable salt thereof, if administered daily for a period of at least 14 days to at least 6 elderly male subjects and at least 6 elderly female subjects in a clinical trial, provides on the last day of such period a mean maximum plasma concentration of befiradol greater than about 35 ng/mL.

Embodiment 27. The method of any one of Embodiments 6-10, 6a, 7a, 8a, 9a-9c, or 10a-10c, wherein the once daily oral dose of befiradol or a pharmaceutically acceptable salt thereof, if administered daily for a period of at least 14 days to at least 6 elderly male subjects and at least 6 elderly female subjects in a clinical trial, provides on the last day of such period a mean maximum plasma concentration of befiradol greater than about 40 ng/mL.

Embodiment 28. The method of any one of Embodiments 6-10, 6a, 7a, 8a, 9a-9c, or 10a-10c, wherein the once daily oral dose of befiradol or a pharmaceutically acceptable salt thereof, if administered daily for a period of at least 14 days to at least 6 elderly male subjects and at least 6 elderly female subjects in a clinical trial, provides on the last day of such period a mean maximum plasma concentration of befiradol greater than about 45 ng/mL.

Embodiment 29. The method of any one of Embodiments 6-10, 6a, 7a, 8a, 9a-9c, or 10a-10c, wherein the once daily oral dose of befiradol or a pharmaceutically acceptable salt thereof, if administered daily for a period of at least 14 days to at least 6 elderly male subjects and at least 6 elderly female subjects in a clinical trial, provides on the last day of such period a mean maximum plasma concentration of befiradol greater than about 50 ng/mL.

Embodiment 30. The method of any one of Embodiments 6-10, 6a, 7a, 8a, 9a-9c, or 10a-10c, wherein the once daily oral dose of befiradol or a pharmaceutically acceptable salt thereof, if administered daily for a period of at least 14 days to at least 6 elderly male subjects and at least 6 elderly female subjects in a clinical trial, provides on the last day of such period a mean maximum plasma concentration of befiradol greater than about 55 ng/mL.

Embodiment 31. The method of any one of Embodiments 6-10, 6a, 7a, 8a, 9a-9c, or 10a-10c, wherein the once daily oral dose of befiradol or a pharmaceutically acceptable salt thereof, if administered daily for a period of at least 14 days to at least 6 elderly male subjects and at least 6 elderly female subjects in a clinical trial, provides on the last day of such period a mean maximum plasma concentration of befiradol greater than about 60 ng/mL.

Embodiment 32. The method of any one of Embodiments 6-10, 6a, 7a, 8a, 9a-9c, or 10a-10c, wherein the once daily oral dose of befiradol or a pharmaceutically acceptable salt thereof, if administered daily for a period of at least 14 days to at least 6 elderly male subjects and at least 6 elderly female subjects in a clinical trial, provides on the last day of such period a mean maximum plasma concentration of befiradol greater than about 65 ng/mL.

Embodiment 33. The method of any one of Embodiments 6-10, 6a, 7a, 8a, 9a-9c, or 10a-10c, wherein the once daily oral dose of befiradol or a pharmaceutically acceptable salt thereof, if administered daily for a period of at least 14 days to at least 6 elderly male subjects and at least 6 elderly female subjects in a clinical trial, provides on the last day of such period a mean maximum plasma concentration of befiradol greater than about 70 ng/mL.

Embodiment 34. The method of any one of Embodiments 15, 15a, 16, or 16a wherein the L-DOPA-related adverse events comprise L-DOPA-induced dyskinesia.

Embodiment 35. The method of any one of Embodiments 13, 13a, 14, or 14a, wherein the method does not significantly reduce anti-parkinsonian efficacy.

Embodiment 36. The method of any one of Embodiments 1-35, 4a, 4b, 5a, 5b, 6a, 7a, 8a, 9a-9c, 10a-10c, 13a, 14a, 15a, or 16a, wherein the once daily oral dose of befiradol or a pharmaceutically acceptable salt thereof is administered in the morning.

Embodiment 37. The method of any one of Embodiments 1-35, 4a, 4b, 5a, 5b, 6a, 7a, 8a, 9a-9c, 10a-10c, 11a, 12a, 13a, 14a, 15a, or 16a, wherein the once daily oral dose of befiradol or a pharmaceutically acceptable salt thereof is administered in the evening.

Embodiment 38. The method of any one of Embodiments 1-37, 4a, 4b, 5a, 5b, 6a, 7a, 8a, 9a-9c, 10a-10c, 11a, 12a, 13a, 14a, 15a, or 16a, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the proportion of patients in the first group who experience an adverse event is not more than 20% higher than the second group.

Embodiment 39. The method of any one of Embodiments 1-37, 4a, 4b, 5a, 5b, 6a, 7a, 8a, 9a-9c, 10a-10c, 11a, 12a, 13a, 14a, 15a, or 16a, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the proportion of patients in the first group who experience an adverse event is not more than 18% higher than the second group.

Embodiment 40. The method of any one of Embodiments 1-37, 4a, 4b, 5a, 5b, 6a, 7a, 8a, 9a-9c, 10a-10c, 11a, 12a, 13a, 14a, 15a, or 16a, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the proportion of patients in the first group who experience an adverse event is not more than 16% higher than the second group.

Embodiment 41. The method of any one of Embodiments 1-37, 4a, 4b, 5a, 5b, 6a, 7a, 8a, 9a-9c, 10a-10c, 11a, 12a, 13a, 14a, 15a, or 16a, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the proportion of patients in the first group who experience an adverse event is not more than 14% higher than the second group.

Embodiment 42. The method of any one of Embodiments 1-37, 4a, 4b, 5a, 5b, 6a, 7a, 8a, 9a-9c, 10a-10c, 11a, 12a, 13a, 14a, 15a, or 16a, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the proportion of patients in the first group who experience an adverse event is not more than 12% higher than the second group.

Embodiment 43. The method of any one of Embodiments 1-37, 4a, 4b, 5a, 5b, 6a, 7a, 8a, 9a-9c, 10a-10c, 11a, 12a, 13a, 14a, 15a, or 16a, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the proportion of patients in the first group who experience an adverse event is not more than 11% higher than the second group.

Embodiment 44. The method of any one of Embodiments 1-43, 4a, 4b, 5a, 5b, 6a, 7a, 8a, 9a-9c, 10a-10c, 11a, 12a, 13a, 14a, 15a, or 16a, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the number of adverse events per patient in the first group is not more than 20% higher than the second group.

Embodiment 45. The method of any one of Embodiments 1-43, 4a, 4b, 5a, 5b, 6a, 7a, 8a, 9a-9c, 10a-10c, 11a, 12a, 13a, 14a, 15a, or 16a, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the number of adverse events per patient in the first group is not more than 18% higher than the second group.

Embodiment 46. The method of any one of Embodiments 1-43, 4a, 4b, 5a, 5b, 6a, 7a, 8a, 9a-9c, 10a-10c, 11a, 12a, 13a, 14a, 15a, or 16a, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the number of adverse events per patient in the first group is not more than 16% higher than the second group.

Embodiment 47. The method of any one of Embodiments 1-43, 4a, 4b, 5a, 5b, 6a, 7a, 8a, 9a-9c, 10a-10c, 11a, 12a, 13a, 14a, 15a, or 16a, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the number of adverse events per patient in the first group is not more than 14% higher than the second group.

Embodiment 48. The method of any one of Embodiments 1-43, 4a, 4b, 5a, 5b, 6a, 7a, 8a, 9a-9c, 10a-10c, 11a, 12a, 13a, 14a, 15a, or 16a, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the number of adverse events per patient in the first group is not more than 12% higher than the second group.

Embodiment 49. The method of any one of Embodiments 1-43, 4a, 4b, 5a, 5b, 6a, 7a, 8a, 9a-9c, 10a-10c, 11a, 12a, 13a, 14a, 15a, or 16a, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the number of adverse events per patient in the first group is not more than 11% higher than the second group.

Embodiment 50. The method of any one of Embodiments 1-49, 4a, 4b, 5a, 5b, 6a, 7a, 8a, 9a-9c, 10a-10c, 11a, 12a, 13a, 14a, 15a, or 16a, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the proportion of patients in the first group who experience dizziness is not more than 20% higher than the second group.

Embodiment 51. The method of any one of Embodiments 1-49, 4a, 4b, 5a, 5b, 6a, 7a, 8a, 9a-9c, 10a-10c, 11a, 12a, 13a, 14a, 15a, or 16a, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the proportion of patients in the first group who experience dizziness is not more than 18% higher than the second group.

Embodiment 52. The method of any one of Embodiments 1-49, 4a, 4b, 5a, 5b, 6a, 7a, 8a, 9a-9c, 10a-10c, 11a, 12a, 13a, 14a, 15a, or 16a, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the proportion of patients in the first group who experience dizziness is not more than 15% higher than the second group.

Embodiment 53. The method of any one of Embodiments 1-49, 4a, 4b, 5a, 5b, 6a, 7a, 8a, 9a-9c, 10a-10c, 11a, 12a, 13a, 14a, 15a, or 16a, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the proportion of patients in the first group who experience dizziness is not more than 12% higher than the second group.

Embodiment 54. The method of any one of Embodiments 1-49, 4a, 4b, 5a, 5b, 6a, 7a, 8a, 9a-9c, 10a-10c, 11a, 12a, 13a, 14a, 15a, or 16a, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the proportion of patients in the first group who experience dizziness is not more than 10% higher than the second group.

Embodiment 55. The method of any one of Embodiments 1-49, 4a, 4b, 5a, 5b, 6a, 7a, 8a, 9a-9c, 10a-10c, 11a, 12a, 13a, 14a, 15a, or 16a, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the proportion of patients in the first group who experience dizziness is not more than 7% higher than the second group.

Embodiment 56. The method of any one of Embodiments 1-49, 4a, 4b, 5a, 5b, 6a, 7a, 8a, 9a-9c, 10a-10c, 11a, 12a, 13a, 14a, 15a, or 16a, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the proportion of patients in the first group who experience dizziness is not more than 5% higher than the second group.

Embodiment 57. A method of treating Parkinson's disease in a patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the method is safe and well tolerated, and
c. the once daily oral dose of befiradol or a pharmaceutically acceptable salt thereof, if administered once to at least 6 male subjects and at least 6 female subjects in a clinical trial without a befiradol dose up-titration period, provides a mean maximum plasma concentration of befiradol greater than about 15 ng/mL.

Embodiment 58. A method of treating Parkinson's disease motor disability in a patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the method is safe and well tolerated, and
c. the once daily oral dose of befiradol or a pharmaceutically acceptable salt thereof, if administered once to at least 6 male subjects and at least 6 female subjects in a clinical trial without a befiradol dose up-titration period, provides a mean maximum plasma concentration of befiradol greater than about 15 ng/mL.

Embodiment 59. A method of treating L-DOPA-induced dyskinesia in a patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the method is safe and well tolerated, and
c. the once daily oral dose of befiradol or a pharmaceutically acceptable salt thereof, if administered once to at least 6 male subjects and at least 6 female subjects in a clinical trial without a befiradol dose up-titration period, provides a mean maximum plasma concentration of befiradol greater than about 15 ng/mL.

Embodiment 60. A method of treating L-DOPA-induced dyskinesia in a patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the once daily oral dose of befiradol or a pharmaceutically acceptable salt thereof, if administered once to at least 6 male subjects and at least 6 female subjects in a clinical trial without a befiradol dose up-titration period, provides a mean maximum plasma concentration of befiradol greater than about 15 ng/ml, and
c. if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the parkinsonian symptoms of the first group significantly improve after the at least 14 days.

Embodiment 60a. A method of treating L-DOPA-induced dyskinesia in a human patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the once daily oral dose of befiradol or a pharmaceutically acceptable salt thereof, when administered once to at least 6 males and at least 6 females in a clinical trial without a befiradol dose up-titration period, provides a mean maximum plasma concentration of befiradol greater than about 15 ng/ml, and
c. if parkinsonian symptoms of at least 15 subjects with Parkinson's disease and L-DOPA-induced dyskinesia are evaluated at baseline and after treatment according to the method for at least 14 days in a double blind, placebo-controlled clinical trial, the parkinsonian symptoms of the treated subjects significantly improve compared to baseline.

Embodiment 60b. A method of treating L-DOPA-induced dyskinesia in a human patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the once daily oral dose of befiradol or a pharmaceutically acceptable salt thereof, when administered once to at least 6 males and at least 6 females in a clinical trial without a befiradol dose up-titration period, provides a mean maximum plasma concentration of befiradol greater than about 15 ng/ml, and
c. if parkinsonian symptoms of at least 15 subjects with Parkinson's disease and L-DOPA-induced dyskinesia are evaluated at baseline and after treatment according to the method for at least 14 days in a double blind, placebo-controlled clinical trial, the parkinsonian symptoms of the treated subjects do not become significantly more severe compared to baseline.

Embodiment 61. A method of treating L-DOPA-induced dyskinesia in a patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the once daily oral dose of befiradol or a pharmaceutically acceptable salt thereof, if administered once to at least 6 male subjects and at least 6 female subjects in a clinical trial without a befiradol dose up-titration period, provides a mean maximum plasma concentration of befiradol greater than about 15 ng/mL, and
c. if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the parkinsonian motor symptoms of the first group significantly improve after the at least 14 days.

Embodiment 61a. A method of treating L-DOPA-induced dyskinesia in a human patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the once daily oral dose of befiradol or a pharmaceutically acceptable salt thereof, when administered once to at least 6 males and at least 6 females in a clinical trial without a befiradol dose up-titration period, provides a mean maximum plasma concentration of befiradol greater than about 15 ng/ml, and
c. if parkinsonian motor symptoms of at least 15 subjects with Parkinson's disease and L-DOPA-induced dyskinesia are evaluated at baseline and after treatment according to the method for at least 14 days in a double blind, placebo-controlled clinical trial, the parkinsonian motor symptoms of the treated subjects significantly improve compared to baseline.

Embodiment 61b. A method of treating L-DOPA-induced dyskinesia in a human patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the once daily oral dose of befiradol or a pharmaceutically acceptable salt thereof, when administered once to at least 6 males and at least 6 females in a clinical trial without a befiradol dose up-titration period, provides a mean maximum plasma concentration of befiradol greater than about 15 ng/mL, and
c. if parkinsonian motor symptoms of at least 15 subjects with Parkinson's disease and L-DOPA-induced dyskinesia are evaluated at baseline and after treatment according to the method for at least 14 days in a double blind, placebo-controlled clinical trial, the parkinsonian motor symptoms of the treated subjects do not become significantly more severe compared to baseline.

Embodiment 62. A method of treating Parkinson's disease in a patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the method is safe and well tolerated, and
c. the once daily oral dose of befiradol or a pharmaceutically acceptable salt thereof, if administered daily for a period of at least 14 days to at least 6 male subjects and at least 6 female subjects in a clinical trial, provides on the last day of such period a mean maximum plasma concentration of befiradol greater than about 30 ng/mL.

Embodiment 62a. A method of treating Parkinson's disease in a human patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the method is safe and well tolerated, and
c. the once daily oral dose of befiradol or a pharmaceutically acceptable salt thereof, when administered daily for a period of at least 14 days to at least 6 males and at least 6 females in a clinical trial, provides on or after day 14 within such period a mean maximum plasma concentration of befiradol greater than about 30 ng/mL.

Embodiment 63. A method of treating Parkinson's disease motor disability in a patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the method is safe and well tolerated, and
c. the once daily oral dose of befiradol or a pharmaceutically acceptable salt thereof, if administered daily for a period of at least 14 days to at least 6 male subjects and at least 6 female subjects in a clinical trial, provides on the last day of such period a mean maximum plasma concentration of befiradol greater than about 30 ng/mL.

Embodiment 63a. A method of treating Parkinson's disease motor disability in a human patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the method is safe and well tolerated, and
c. the once daily oral dose of befiradol or a pharmaceutically acceptable salt thereof, when administered daily for a period of at least 14 days to at least 6 males and at least 6 females in a clinical trial, provides on or after day 14 within such period a mean maximum plasma concentration of befiradol greater than about 30 ng/mL.

Embodiment 64. A method of treating L-DOPA-induced dyskinesia in a patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the method is safe and well tolerated, and
c. the once daily oral dose of befiradol or a pharmaceutically acceptable salt thereof, if administered daily for a period of at least 14 days to at least 6 male subjects and at least 6 female subjects in a clinical trial, provides on the last day of such period a mean maximum plasma concentration of befiradol greater than about 30 ng/mL.

Embodiment 64a. A method of treating L-DOPA-induced dyskinesia in a human patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the method is safe and well tolerated, and
c. the once daily oral dose of befiradol or a pharmaceutically acceptable salt thereof, when administered daily for a period of at least 14 days to at least 6 males and at least 6 females in a clinical trial, provides on or after day 14 within such period a mean maximum plasma concentration of befiradol greater than about 30 ng/mL.

Embodiment 65. A method of treating L-DOPA-induced dyskinesia in a patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the once daily oral dose of befiradol or a pharmaceutically acceptable salt thereof, if administered daily for a period of at least 14 days to at least 6 male subjects and at least 6 female subjects in a clinical trial, provides on the last day of such period a mean maximum plasma concentration of befiradol greater than about 30 ng/ml, and
c. if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the parkinsonian symptoms of the first group significantly improve after the at least 14 days.

Embodiment 65a. A method of treating L-DOPA-induced dyskinesia in a human patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the once daily oral dose of befiradol or a pharmaceutically acceptable salt thereof, when administered daily for a period of at least 14 days to at least 6 males and at least 6 females in a clinical trial, provides on or after day 14 within such period a mean maximum plasma concentration of befiradol greater than about 30 ng/ml, and
c. if parkinsonian symptoms of at least 15 subjects with Parkinson's disease and L-DOPA-induced dyskinesia are evaluated at baseline and after treatment according to the method for at least 14 days in a double blind, placebo-controlled clinical trial, the parkinsonian symptoms of the treated subjects significantly improve compared to baseline.

Embodiment 65b. A method of treating L-DOPA-induced dyskinesia in a human patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the once daily oral dose of befiradol or a pharmaceutically acceptable salt thereof, when administered daily for a period of at least 14 days to at least 6 males and at least 6 females in a clinical trial, provides on or after day 14 within such period a mean maximum plasma concentration of befiradol greater than about 30 ng/mL, and
c. if parkinsonian symptoms of at least 15 subjects with Parkinson's disease and L-DOPA-induced dyskinesia are evaluated at baseline and after treatment according to the method for at least 14 days in a double blind, placebo-controlled clinical trial, the parkinsonian symptoms of the treated subjects do not become significantly more severe compared to baseline.

Embodiment 65c. A method of treating L-DOPA-induced dyskinesia in a human patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the once daily oral dose of befiradol or a pharmaceutically acceptable salt thereof, when administered daily for a period of at least 14 days to at least 6 males and at least 6 females in a clinical trial, provides on or after day 14 within such period a mean maximum plasma concentration of befiradol greater than about 30 ng/ml, and
c. if parkinsonian symptoms are evaluated at baseline and after treatment in a double blind, placebo-controlled clinical trial in which a first group comprising at least 15 subjects with Parkinson's disease and L-DOPA-induced dyskinesia is treated according to the method for at least 14 days and a second group comprising at least 7 subjects with Parkinson's disease and L-DOPA-induced dyskinesia is treated according to the method for the at least 14 days but using placebo instead of befiradol or a pharmaceutically acceptable salt thereof, the parkinsonian symptoms of the first group, but not the second group, significantly improve compared to baseline.

Embodiment 66. A method of treating L-DOPA-induced dyskinesia in a patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the once daily oral dose of befiradol or a pharmaceutically acceptable salt thereof, if administered daily for a period of at least 14 days to at least 6 male subjects and at least 6 female subjects in a clinical trial, provides on the last day of such period a mean maximum plasma concentration of befiradol greater than about 30 ng/ml, and
c. if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the parkinsonian motor symptoms of the first group significantly improve after the at least 14 days.

Embodiment 66a. A method of treating L-DOPA-induced dyskinesia in a human patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the once daily oral dose of befiradol or a pharmaceutically acceptable salt thereof, when administered daily for a period of at least 14 days to at least 6 males and at least 6 females in a clinical trial, provides on or after day 14 within such period a mean maximum plasma concentration of befiradol greater than about 30 ng/ml, and
c. if parkinsonian motor symptoms of at least 15 subjects with Parkinson's disease and L-DOPA-induced dyskinesia are evaluated at baseline and after treatment according to the method for at least 14 days in a double blind, placebo-controlled clinical trial, the parkinsonian motor symptoms of the treated subjects significantly improve compared to baseline.

Embodiment 66b. A method of treating L-DOPA-induced dyskinesia in a human patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the once daily oral dose of befiradol or a pharmaceutically acceptable salt thereof, when administered daily for a period of at least 14 days to at least 6 males and at least 6 females in a clinical trial, provides on or after day 14 within such period a mean maximum plasma concentration of befiradol greater than about 30 ng/ml, and
c. if parkinsonian motor symptoms of at least 15 subjects with Parkinson's disease and L-DOPA-induced dyskinesia are evaluated at baseline and after treatment according to the method for at least 14 days in a double blind, placebo-controlled clinical trial, the parkinsonian motor symptoms of the treated subjects do not become significantly more severe compared to baseline.

Embodiment 66c. A method of treating L-DOPA-induced dyskinesia in a human patient in need thereof, comprising administering to the patient a therapeutically effective amount of L-DOPA and befiradol or a pharmaceutically acceptable salt thereof, wherein:
a. the therapeutically effective amount of befiradol or a pharmaceutically acceptable salt thereof is administered to the patient as a once daily oral dose,
b. the once daily oral dose of befiradol or a pharmaceutically acceptable salt thereof, when administered daily for a period of at least 14 days to at least 6 males and at least 6 females in a clinical trial, provides on or after day 14 within such period a mean maximum plasma concentration of befiradol greater than about 30 ng/ml, and
c. if parkinsonian motor symptoms are evaluated at baseline and after treatment in a double blind, placebo-controlled clinical trial in which a first group comprising at least 15 subjects with Parkinson's disease and L-DOPA-induced dyskinesia is treated according to the method for at least 14 days and a second group comprising at least 7 subjects with Parkinson's disease and L-DOPA-induced dyskinesia is treated according to the method for the at least 14 days but using placebo instead of befiradol or a pharmaceutically acceptable salt thereof, the parkinsonian motor symptoms of the first group, but not the second group, significantly improve compared to baseline.

Embodiment 67. The method of any one of Embodiments 9c, 10c, 57-61, 60a, 60b, 61a, or 61b, wherein the once daily oral dose of befiradol or a pharmaceutically acceptable salt thereof, if administered once to at least 6 male subjects and at least 6 female subjects in a clinical trial without a befiradol dose up-titration period, provides a mean maximum plasma concentration of befiradol greater than about 20 ng/mL.

Embodiment 68. The method of any one of Embodiments 9c, 10c, 57-61, 60a, 60b, 61a, or 61b, wherein the once daily oral dose of befiradol or a pharmaceutically acceptable salt thereof, if administered once to at least 6 male subjects and at least 6 female subjects in a clinical trial without a befiradol dose up-titration period, provides a mean maximum plasma concentration of befiradol greater than about 21 ng/mL.

Embodiment 69. The method of any one of Embodiments 9c, 10c, 57-61, 60a, 60b, 61a, or 61b, wherein the once daily oral dose of befiradol or a pharmaceutically acceptable salt thereof, if administered once to at least 6 male subjects and at least 6 female subjects in a clinical trial without a befiradol dose up-titration period, provides a mean maximum plasma concentration of befiradol greater than about 22 ng/mL.

Embodiment 70. The method of any one of Embodiments 9c, 10c, 57-61, 60a, 60b, 61a, or 61b, wherein the once daily oral dose of befiradol or a pharmaceutically acceptable salt thereof, if administered once to at least 6 male subjects and at least 6 female subjects in a clinical trial without a befiradol dose up-titration period, provides a mean maximum plasma concentration of befiradol greater than about 23 ng/mL.

Embodiment 71. The method of any one of Embodiments 9c, 10c, 57-61, 60a, 60b, 61a, or 61b, wherein the once daily oral dose of befiradol or a pharmaceutically acceptable salt thereof, if administered once to at least 6 male subjects and at least 6 female subjects in a clinical trial without a befiradol dose up-titration period, provides a mean maximum plasma concentration of befiradol greater than about 24 ng/mL.

Embodiment 72. The method of any one of Embodiments 9c, 10c, 57-61, 60a, 60b, 61a, or 61b, wherein the once daily oral dose of befiradol or a pharmaceutically acceptable salt thereof, if administered once to at least 6 male subjects and at least 6 female subjects in a clinical trial without a befiradol dose up-titration period, provides a mean maximum plasma concentration of befiradol greater than about 25 ng/mL.

Embodiment 73. The method of any one of Embodiments 9c, 10c, 57-61, 60a, 60b, 61a, or 61b, wherein the once daily oral dose of befiradol or a pharmaceutically acceptable salt thereof, if administered once to at least 6 male subjects and at least 6 female subjects in a clinical trial without a befiradol dose up-titration period, provides a mean maximum plasma concentration of befiradol greater than about 30 ng/mL.

Embodiment 73a. The method of any one of Embodiments 9c, 10c, 60a, 60b, 61a, or 61b, wherein the at least 6 males and at least 6 females have Parkinson's disease.

Embodiment 73b. The method of any one of Embodiments 57-61 or 67-73, wherein the at least 6 male subjects and at least 6 female subjects have Parkinson's disease.

Embodiment 73c. The method of any one of Embodiments 9c, 10c, 60a, 60b, 61a, or 61b, wherein the at least 6 males and at least 6 females are aged 50 or older.

Embodiment 73d. The method of any one of Embodiments 57-61 or 67-73, wherein the at least 6 male subjects and at least 6 female subjects are aged 50 or older.

Embodiment 74. The method of any one of Embodiments 62-66, 62a, 63a, 64a, 65a-65c, or 66a-66c, wherein the once daily oral dose of befiradol or a pharmaceutically acceptable salt thereof, if administered daily for a period of at least 14 days to at least 6 male subjects and at least 6 female subjects in a clinical trial, provides on the last day of such period a mean maximum plasma concentration of befiradol greater than about 35 ng/mL.

Embodiment 75. The method of any one of Embodiments 62-66, 62a, 63a, 64a, 65a-65c, or 66a-66c, wherein the once daily oral dose of befiradol or a pharmaceutically acceptable salt thereof, if administered daily for a period of at least 14 days to at least 6 male subjects and at least 6 female subjects in a clinical trial, provides on the last day of such period a mean maximum plasma concentration of befiradol greater than about 40 ng/mL.

Embodiment 76. The method of any one of Embodiments 62-66, 62a, 63a, 64a, 65a-65c, or 66a-66c, wherein the once daily oral dose of befiradol or a pharmaceutically acceptable salt thereof, if administered daily for a period of at least 14 days to at least 6 male subjects and at least 6 female subjects in a clinical trial, provides on the last day of such period a mean maximum plasma concentration of befiradol greater than about 45 ng/mL.

Embodiment 77. The method of any one of Embodiments 62-66, 62a, 63a, 64a, 65a-65c, or 66a-66c, wherein the once daily oral dose of befiradol or a pharmaceutically acceptable salt thereof, if administered daily for a period of at least 14 days to at least 6 male subjects and at least 6 female subjects in a clinical trial, provides on the last day of such period a mean maximum plasma concentration of befiradol greater than about 50 ng/mL.

Embodiment 78. The method of any one of Embodiments 62-66, 62a, 63a, 64a, 65a-65c, or 66a-66c, wherein the once daily oral dose of befiradol or a pharmaceutically acceptable salt thereof, if administered daily for a period of at least 14 days to at least 6 male subjects and at least 6 female subjects in a clinical trial, provides on the last day of such period a mean maximum plasma concentration of befiradol greater than about 55 ng/mL.

Embodiment 79. The method of any one of Embodiments 62-66, 62a, 63a, 64a, 65a-65c, or 66a-66c, wherein the once daily oral dose of befiradol or a pharmaceutically acceptable salt thereof, if administered daily for a period of at least 14 days to at least 6 male subjects and at least 6 female subjects in a clinical trial, provides on the last day of such period a mean maximum plasma concentration of befiradol greater than about 60 ng/mL.

Embodiment 80. The method of any one of Embodiments 62-66, 62a, 63a, 64a, 65a-65c, or 66a-66c, wherein the once daily oral dose of befiradol or a pharmaceutically acceptable salt thereof, if administered daily for a period of at least 14 days to at least 6 male subjects and at least 6 female subjects in a clinical trial, provides on the last day of such period a mean maximum plasma concentration of befiradol greater than about 65 ng/mL.

Embodiment 81. The method of any one of Embodiments 62-66, 62a, 63a, 64a, 65a-65c, or 66a-66c, wherein the once daily oral dose of befiradol or a pharmaceutically acceptable salt thereof, if administered daily for a period of at least 14 days to at least 6 male subjects and at least 6 female subjects in a clinical trial, provides on the last day of such period a mean maximum plasma concentration of befiradol greater than about 70 ng/mL.

Embodiment 81a. The method of any one of Embodiments 62a, 63a, 64a, 65a-65c, or 66a-66c, wherein the at least 6 males and at least 6 females have Parkinson's disease.

Embodiment 81b. The method of any one of Embodiments 62-66 or 74-81, wherein the at least 6 male subjects and at least 6 female subjects have Parkinson's disease.

Embodiment 81c. The method of any one of Embodiments 62a, 63a, 64a, 65a-65c, or 66a-66c, wherein the at least 6 males and at least 6 females are aged 50 or older.

Embodiment 81d. The method of any one of Embodiments 62-66 or 74-81, wherein the at least 6 male subjects and at least 6 female subjects are aged 50 or older.

Embodiment 82. The method of any one of Embodiments 57-81, 60a, 60b, 61a, 61b, 62a, 63a, 64a, 65a-65c, 66a-66c, 73a-73d, or 81a-81d, wherein the once daily oral dose of befiradol or a pharmaceutically acceptable salt thereof is administered in the morning.

Embodiment 83. The method of any one of Embodiments 57-81, 60a, 60b, 61a, 61b, 62a, 63a, 64a, 65a-65c, 66a-66c, 73a-73d, or 81a-81d, wherein the once daily oral dose of befiradol or a pharmaceutically acceptable salt thereof is administered in the evening.

Embodiment 84. The method of any one of Embodiments 57-83, 60a, 60b, 61a, 61b, 62a, 63a, 64a, 65a-65c, 66a-66c, 73a-73d, or 81a-81d, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the proportion of patients in the first group who experience an adverse event is not more than 20% higher than the second group.

Embodiment 85. The method of any one of Embodiments 57-83, 60a, 60b, 61a, 61b, 62a, 63a, 64a, 65a-65c, 66a-66c, 73a-73d, or 81a-81d, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the proportion of patients in the first group who experience an adverse event is not more than 18% higher than the second group.

Embodiment 86. The method of any one of Embodiments 57-83, 60a, 60b, 61a, 61b, 62a, 63a, 64a, 65a-65c, 66a-66c, 73a-73d, or 81a-81d, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the proportion of patients in the first group who experience an adverse event is not more than 16% higher than the second group.

Embodiment 87. The method of any one of Embodiments 57-83, 60a, 60b, 61a, 61b, 62a, 63a, 64a, 65a-65c, 66a-66c, 73a-73d, or 81a-81d, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the proportion of patients in the first group who experience an adverse event is not more than 14% higher than the second group.

Embodiment 88. The method of any one of Embodiments 57-83, 60a, 60b, 61a, 61b, 62a, 63a, 64a, 65a-65c, 66a-66c, 73a-73d, or 81a-81d, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the proportion of patients in the first group who experience an adverse event is not more than 12% higher than the second group.

Embodiment 89. The method of any one of Embodiments 57-83, 60a, 60b, 61a, 61b, 62a, 63a, 64a, 65a-65c, 66a-66c, 73a-73d, or 81a-81d, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the proportion of patients in the first group who experience an adverse event is not more than 11% higher than the second group.

Embodiment 90. The method of any one of Embodiments 57-89, 60a, 60b, 61a, 61b, 62a, 63a, 64a, 65a-65c, 66a-66c, 73a-73d, or 81a-81d, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the number of adverse events per patient in the first group is not more than 20% higher than the second group.

Embodiment 91. The method of any one of Embodiments 57-89, 60a, 60b, 61a, 61b, 62a, 63a, 64a, 65a-65c, 66a-66c, 73a-73d, or 81a-81d, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the number of adverse events per patient in the first group is not more than 18% higher than the second group.

Embodiment 92. The method of any one of Embodiments 57-89, 60a, 60b, 61a, 61b, 62a, 63a, 64a, 65a-65c, 66a-66c, 73a-73d, or 81a-81d, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the number of adverse events per patient in the first group is not more than 16% higher than the second group.

Embodiment 93. The method of any one of Embodiments 57-89, 60a, 60b, 61a, 61b, 62a, 63a, 64a, 65a-65c, 66a-66c, 73a-73d, or 81a-81d, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the number of adverse events per patient in the first group is not more than 14% higher than the second group.

Embodiment 94. The method of any one of Embodiments 57-89, 60a, 60b, 61a, 61b, 62a, 63a, 64a, 65a-65c, 66a-66c, 73a-73d, or 81a-81d, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the number of adverse events per patient in the first group is not more than 12% higher than the second group.

Embodiment 95. The method of any one of Embodiments 57-89, 60a, 60b, 61a, 61b, 62a, 63a, 64a, 65a-65c, 66a-66c, 73a-73d, or 81a-81d, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the number of adverse events per patient in the first group is not more than 11% higher than the second group.

Embodiment 96. The method of any one of Embodiments 57-95, 60a, 60b, 61a, 61b, 62a, 63a, 64a, 65a-65c, 66a-66c, 73a-73d, or 81a-81d, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the proportion of patients in the first group who experience dizziness is not more than 20% higher than the second group.

Embodiment 97. The method of any one of Embodiments 57-95, 60a, 60b, 61a, 61b, 62a, 63a, 64a, 65a-65c, 66a-66c, 73a-73d, or 81a-81d, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the proportion of patients in the first group who experience dizziness is not more than 18% higher than the second group.

Embodiment 98. The method of any one of Embodiments 57-95, 60a, 60b, 61a, 61b, 62a, 63a, 64a, 65a-65c, 66a-66c, 73a-73d, or 81a-81d, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the proportion of patients in the first group who experience dizziness is not more than 15% higher than the second group.

Embodiment 99. The method of any one of Embodiments 57-95, 60a, 60b, 61a, 61b, 62a, 63a, 64a, 65a-65c, 66a-66c, 73a-73d, or 81a-81d, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the proportion of patients in the first group who experience dizziness is not more than 12% higher than the second group.

Embodiment 100. The method of any one of Embodiments 57-95, 60a, 60b, 61a, 61b, 62a, 63a, 64a, 65a-65c, 66a-66c, 73a-73d, or 81a-81d, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the proportion of patients in the first group who experience dizziness is not more than 10% higher than the second group.

Embodiment 101. The method of any one of Embodiments 57-95, 60a, 60b, 61a, 61b, 62a, 63a, 64a, 65a-65c, 66a-66c, 73a-73d, or 81a-81d, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the proportion of patients in the first group who experience dizziness is not more than 7% higher than the second group.

Embodiment 102. The method of any one of Embodiments 57-95, 60a, 60b, 61a, 61b, 62a, 63a, 64a, 65a-65c, 66a-66c, 73a-73d, or 81a-81d, wherein if a first group comprising at least 15 patients is treated according to the method for at least 14 days in a double blind, placebo-controlled clinical trial and in the clinical trial a second group comprising at least 7 other patients is treated according to the method for at least 14 days but using placebo instead of befiradol or a pharmaceutically acceptable salt thereof, the proportion of patients in the first group who experience dizziness is not more than 5% higher than the second group.

### VII. Examples

The following examples illustrate certain aspects and embodiments of the present disclosure and are not intended to limit the scope of the claims.

### Example 1—a Randomized, Double-Blind, Placebo-Controlled Study to Assess the Safety, Tolerability and Preliminary Efficacy of NLX-112 Versus Placebo in Levodopa-Induced Dyskinesia in Parkinson's Disease

### Study Design

This was an 8-week, double-blind, randomized, placebo-controlled Phase 2a proof-of-concept study evaluating the safety, tolerability, and preliminary efficacy of up to 2 mg/day (1 mg BID) of NLX-112 versus placebo in patients with moderate to severe L-DOPA-induced dyskinesia (LID) in Parkinson's disease (PD) with up- and down-titration. NLX-112 was up-titrated to either 2 mg/day or to the highest well-tolerated dose less than 2 mg/day over 4 weeks, maintained at the well-tolerated dose for an additional 2 weeks, and then down-titrated over 2 weeks. The study was performed at 5 clinical sites in Sweden (NCT05148884).

### Objectives

Primary objective: To evaluate the safety and tolerability of NLX-112, titrated up to a maximum of 2 mg/day, compared to placebo during 8 weeks of daily treatment in PD patients with LID. Safety/tolerability assessments included: medical history (screening), physical examination (screening and follow-up), 12-lead electrocardiogram (ECG, all clinic visits), vital signs (all clinic visits), frequency, intensity and seriousness of AEs (from baseline visit through follow up visit), assessment of suicidal ideation/behavior by the Columbia Suicide Severity Rating Scale (C-SSRS, all clinic visits), and clinical labs (hematology, blood chemistry, and urinalysis, all clinic visits).

Secondary objective: To assess the preliminary efficacy of NLX-112 treatment on motor symptoms-reducing troublesome LID and its effects on Parkinsonism. Patients were rated for dyskinesia at baseline and on days 28 (at end of up-titration period) and 42 (end of stable dosing period). Because the level of dyskinesia experienced by patients can vary over time, they were given 150% (i.e., 1.5×) of their regular L-DOPA dose (up to a maximum of 250 mg) as a challenge 30 min before each dyskinesia assessment to improve comparability between patients and between visits. Patients were filmed 30, 60 and 90 min after dosing with L-DOPA (˜5 min of filming each time). LID was assessed using the Unified Dyskinesia Rating Scale (UDysRS). Changes from baseline are shown for total score (i.e., Parts 1 to 4), for total objective score (i.e., Parts 3+4), and for the sum of Part 3 objective impairment assessment scores. Because objective LID assessment was carried out at 3 time-points (30, 60 and 90 min) following L-DOPA challenge, the above parameters were analyzed based on the most severe of the three scores as well as on the average score of the 3 time points. PD symptoms were assessed by Unified Parkinson's Disease Rating Scale (UPDRS) 30-90 min after the 150% L-DOPA challenge (i.e., when the patient was ON). Changes from baseline are shown for total score (i.e., Parts 1 to 4) and individual scores for each Part, 1, 2, 3 and 4. A PD Home Dyskinesia eDiary was completed by the patient and/or caregiver for two consecutive 24-hour periods prior to clinic visits on days 28 and 42. The outcome was change from baseline in ‘Good ON Time’ (i.e., ON Without Dyskinesia plus ON With Non-troublesome Dyskinesia).

Exploratory Objectives: To assess the preliminary efficacy of NLX-112 treatment in improving selected non-motor symptoms of PD, including pain assessed by the KPPS, bladder function assessed by the ICIQ-OAB, sleep function assessed by the ESS, mood assessed by the HADS and quality of living with PD assessed by the PDQ39. To collect blood samples for potential NLX-112 plasma concentration analysis. Clinical Global Impression of Severity (CGI-S), and Clinical Global Impression of Change (CGI C).

### Endpoints

### Primary Endpoints:

Frequency, intensity and seriousness of adverse events (AEs).
Clinically significant changes from baseline in electrocardiogram (ECG), vital signs, safety laboratory parameters and physical examinations.
Suicidal ideation/behavior as assessed by the Columbia Suicide Severity Rating Scale (C-SSRS).

### Secondary Endpoints:

Change from baseline at the final efficacy clinic visit (Day 42) in the Unified Dyskinesia Rating Scale (UDysRS) total score [Timeframe: Baseline to Day 42]. Patients were challenged with 150% of their standard L-DOPA dose (maximum L-DOPA dose 250 mg) 30 minutes prior to the first UDysRS assessment.
Change from baseline in UDysRS total score at Day 28 and Day 42, after a 150% L-DOPA dose challenge.
Change from baseline in total objective score (Parts III, IV) of the UDysRS at Day 28 and Day 42, after a 150% L-DOPA dose challenge.
Change from baseline in ON Time Without Troublesome Dyskinesia (ON Without Dyskinesia plus ON With Non-troublesome Dyskinesia) based on a PD Home Dyskinesia Diary.
Change from baseline in Unified Parkinson's Disease Rating Scale (UPDRS) scores (Part III, motor examination).
Change from baseline in UPDRS combined scores (Parts I, II, III and IV).
Clinical Global Impression of Change (CGI-C) in overall PD symptoms
Change from baseline in dyskinesia scores measured by the Kinesia 360 (Great Lakes Neurotechnologies, Inc) wearable dyskinesia assessment system.

### Exploratory Endpoints:

Change from baseline in the King's Parkinson's Disease Pain Scale (KPPS) total score and in each of the 7 pain domain sub-scores.
Change from baseline in the Parkinson's Disease Questionnaire (PDQ39) total score and domain scores.
Change from baseline in the Hospital Anxiety Depression Scale (HADS)
Change from baseline in the International Consultation on Incontinence Questionnaire (ICIQ) Overactive Bladder Module (ICIQ-OAB) total score.
Change from baseline in the Epworth Sleepiness Scale (ESS).
Plasma concentrations of NLX-112.

### Number of Patients

A total of 27 patients were randomized at 5 clinical sites. 22 patients (15 on NLX-112, 7 on placebo) completed the 8-week treatment according to the protocol.

### Inclusion Criteria

To be eligible to participate in the study, a patient must meet ALL of the following inclusion criteria:
Patient is 30-85 years old (inclusive) with a diagnosis of idiopathic PD according to the UK Parkinson's Disease Society Brain Bank Clinical Diagnosis criteria.
PD patient is stably and optimally treated with L-DOPA; other anti-PD treatments are allowed if used for at least 4 weeks of previous continuous treatment (amantadine continuation allowed). All antiparkinsonian medications, including L-DOPA preparations, unchanged for at least 30 days prior to screening.
Patient agrees to be challenged with 150% of their normal L-DOPA dose (maximum L-DOPA dose 250 mg) 30 minutes prior to efficacy assessments at baseline (Visit 2) and at the 2 efficacy clinic visits (Visits 6 and 7).
PD patient exhibits troublesome peak-dose LID, confirmed by a score of at least 1 on PartIV, item 33 (disability) of the Unified Parkinson's Disease Rating Scale (UPDRS) at screening (Visit 1) and at Day 1 (baseline, Visit 2).
At least 90 minutes in total for each 24-hour period during 2 days are indicated as “ON with troublesome dyskinesia” (according to the PD Home Dyskinesia Diary) prior to Day 1 (baseline, Visit 2).
Patient (and/or caregiver) demonstrates ability to accurately complete the PD Home Dyskinesia Diary entries during the screening visit.
Patient can read well enough to understand the informed consent document and other subject materials.
Female patients of child-bearing potential must have a negative urine pregnancy test at screening (Visit 1) and on Day 1 (Visit 2), must agree to avoid pregnancy during the study, and must practice abstinence (only allowed when this is the preferred and usual lifestyle of the subject) or must agree to use a highly effective method of contraception with a failure rate of <1% to prevent pregnancy (combined [oestrogen and progestogen containing] hormonal contraception associated with inhibition of ovulation [oral, intravaginal, transdermal], progestogen-only hormonal contraception associated with inhibition of ovulation [oral, injectable, implantable], intrauterine device [IUD] or intrauterine hormone-releasing system [IUS]) starting from 4 weeks prior to administration of the study drug and continuing during the course of the study until 4 weeks after last investigational medicinal product (IMP) administration. Female subjects must agree to refrain from donating eggs from the date of dosing until 3 months after dosing with the IMP. Their male partner must agree to use a condom during the same time frame if he has not undergone vasectomy.
Females of non-childbearing potential are defined as pre-menopausal females who are sterilized (tubal ligation or permanent bilateral occlusion of fallopian tubes); or females who have undergone hysterectomy or bilateral oophorectomy; or post-menopausal defined as 12 months of amenorrhea (in questionable cases a blood sample with detection of follicle stimulating hormone [FSH] 25-140 IE/L is confirmatory).
Male patients must be either vasectomized, consent to use condom or practice sexual abstinence to prevent pregnancy and drug exposure of a partner and refrain from donating sperm from the date of dosing until 3 months after dosing with the IMP. Their female partner of child-bearing potential must use highly effective contraceptive methods with a failure rate of <1% to prevent pregnancy (see above) during the same period.

### Exclusion Criteria

If the patient meets any of the following criteria, the patient MUST NOT be enrolled:
Patient has severe PD with a Hoehn and Yahr stage=5.
Patient has unstable medical status, prior brain surgery against tumors or haemorrhage (excluding deep brain stimulation [DBS], i.e., DBS patients would be allowed to be enrolled) or is scheduled to receive surgery during the trial period.
Patient has orthostatic hypotension: a decrease in systolic blood pressure (at least 20 mm Hg) or diastolic blood pressure (at least 10 mm Hg) within 3 minutes of the patient standing up, compared to pressures obtained while in a sitting position for at least 5 minutes. At screening and baseline visits (Visit 1 and Visit 2), vital signs to assess orthostatic hypotension would be conducted in triplicate, 15-20 minutes apart, with the average of the 3 assessments used for exclusion.
Patient has dementia (MMSE <20).
Patient has clinically significant renal or liver disorder.
Patient has history of seizures.
Patient had previous surgery for PD.
Patient has prolonged QTcF (>450 ms), cardiac arrhythmias, or any clinically significant abnormalities in the resting ECG at the time of screening.
Patient has ongoing or history of severe allergy/hypersensitivity; or history of hypersensitivity to drugs with a similar chemical structure or class to NLX-112.
Patient currently exhibits generalized obsessive-compulsive disorder, panic disorder, bipolar disorder, post-traumatic stress syndrome (PTSD), clinically significant parasomnias or any other psychotic disorder as established by structured clinical interview for DSM disorders (SCID). Visual hallucinations are allowed.
Any suicidal actions in the past 2 years (per investigator judgement i.e., actual attempt, interrupted attempt, aborted attempt, or preparatory acts or behavior).
Any suicidal ideation of type 4 or 5 in the C-SSRS in the past 3 months (i.e., active suicidal thought with intent but without specific plan, or active suicidal thought with plan and intent).
Patient has taken an anti-convulsant, an anti-psychotic (except quetiapine), pindolol, tertatolol or buspirone within 4 weeks of baseline (Day 1, Visit 2).
Patient has taken, within 4 weeks of baseline (Day 1, Visit 2), any medication that inhibits or up-regulates CYP450 3A4.
Patient is concurrently participating in another investigational drug trial or has participated in another investigational drug trial within the past 3 months.
Patient is at high risk of non-compliance in the Investigator's opinion.
Patient has abnormal ECG.

### Restrictions During the Study

The patients must be willing to comply to the following restrictions during the entire study duration i.e., from screening to the end-of-study visit.

### General Restrictions

All females of child-bearing potential must use highly effective contraception (defined in inclusion criteria) or practice abstinence during the study and for 4 weeks after IMP administration and refrain from donating eggs from the date of dosing until 3 months after dosing with the IMP. Their male partner is expected to use a condom during the same time frame if he has not undergone vasectomy.

The male patients must be either vasectomised, consent to use condom or practice sexual abstinence to prevent pregnancy and drug exposure of a partner and refrain from donating sperm from the date of dosing until 3 months after dosing with the IMP. Their fertile female partner is expected to use highly effective contraceptive methods to prevent pregnancy (for details, refer to inclusion criteria) during the same period.

The IMPs should be taken during a meal and swallowed with approximately 240 mL of tap water.

Consumption of grapefruit and/or grapefruit containing products or star fruit is not allowed during the study.

The patients must not donate blood or plasma during the study until 3 months after the final medical examination at the end-of-study visit.

### Prior and Concomitant Therapy

Use of an anti-convulsant, an anti-psychotic (except quetiapine), pindolol, tertatolol or buspirone within 4 weeks of baseline (Day 1, Visit 2) and during the study is prohibited.

In addition, the following drugs which may affect CYP450 3A4 activity are prohibited from 4 weeks prior to baseline (Day 1, Visit 2) and during the study:
Inducers: barbiturates, carbamazepine, efavirenz, glucocorticoids, modafinil, nevirapine, phenobarbital, phenytoin, rifampin, Saint-John's wort, troglitazone, oxcarbazepine, pioglitazone, rifabutin.
Inhibitors: amiodarone, aprepitant, chloramphenicol, cimetidine, clarithromycin, delaviridine, diethyl-dithiocarbamate, diltiazem, erythromycin, fluconazole, fluvoxamine, gestodene, imatinib, indinavir, itraconazole, ketoconazole, mifepristone, nefazodone, nelfinavir, norfloxacine, norfluoxetine, mibefradil, verapamil, ritonavir, voriconazole.

Prohibited concomitant medications also include those, which in the Investigator's opinion (with Sponsor agreement), may negatively interact with the study medication NLX-112 and lead to untoward side effects or otherwise affect patient safety.

Medications considered necessary for the patient's safety and wellbeing may be given at the discretion of the Investigator. Following consultation with the Sponsor, the Investigator will determine whether or not the patient should continue in the study.

Use of selective serotonin reuptake inhibitors (SSRIs), benzodiazepines and amantadine are allowed as is occasional use of apomorphine as judged by the Investigator. In addition, Covid-19 vaccination is allowed.

### Patient Withdrawal

### General Withdrawal Criteria

Patients were free to discontinue their participation in the study at any time and for whatever reason without affecting their right to an appropriate follow-up investigation or their future care. If possible, the reason for withdrawal of consent would be documented.

Patients could be discontinued from the study at any time at the discretion of the Investigator. Reasons for discontinuation include:
d. Patient decision (withdrawal of consent)
e. Severe non-compliance to study protocol procedures, as judged by the Investigator and/or Sponsor.
f. Permanent discontinuation of IMP. Temporarily treatment interruptions of ≥3 days may result in study discontinuation as judged by the Investigator and Sponsor.
g. Patient is lost to follow-up. If a patient misses a scheduled visit and is repeatedly unable to be contacted, the patient would be withdrawn from the study.
h. Significant AEs posing a risk for the patient, as judged by the Investigator and/or Sponsor.
i. Withdrawal of informed consent to the use of biological samples
j. Pregnancy
k. Death-Request an autopsy report.
l. Use of prohibited medication as judged by the Investigator and/or Sponsor.
m. The patient exhibits serious suicidality, in the clinical judgement of the investigator or according to criteria below:
n. Any suicidal behavior (i.e., actual attempt, interrupted attempt, aborted attempt, or preparatory acts or behavior)
o. Any suicidal ideation of type 4 or 5 in the C-SSRS (i.e., active suicidal thought with intent but without specific plan, or active suicidal thought with plan and intent)

### QTc Withdrawal Criteria

A patient meeting the criteria below would be withdrawn from the study. The same QT correction formula would be used to determine discontinuation throughout the study.
p. QTcF >500 msec
q. Change from baseline: QTc >60 ms

Withdrawal decisions would be based on an average QTc value of triplicate ECGs. If an ECG demonstrated a prolonged QT interval, 2 more ECGs would be obtained over a brief period and the averaged QTc values of the 3 ECGs used to determine whether the patient should be discontinued from the study.

### Liver Chemistry Withdrawal Criteria

Liver chemistry threshold stopping criteria have been designed to assure patient safety and to evaluate liver event etiology. Study treatment would be stopped for the individual patient if any of the following liver chemistry stopping criteria, defined in the U.S. Food and Drug Administration (FDA) Guidance on Drug-Induced Liver Injury, is met:
Alanine aminotransferase (ALT) 3×upper limit of normal (ULN) and total bilirubin≥2×ULN (>35% direct bilirubin); or ALT 3×ULN and INR>1.5)
NOTE: plasma bilirubin fractionation would be performed. Bilirubin is also measured via urine dipstick (a measurement of direct bilirubin, which would suggest liver injury).
ALT 5×ULN
ALT 3×ULN if associated with symptoms (new or worsening) believed to be related to hepatitis (such as fatigue, nausea, vomiting, right upper quadrant pain or tenderness or jaundice) or believed to be related to hypersensitivity (such as fever, rash or eosinophilia).
Patients with ALT 3×ULN and <5×ULN and bilirubin<2×ULN, who do not exhibit hepatitis symptoms or rash, would be allowed to continue study treatment as long as they are monitored weekly for 4 weeks.

Withdrawal decisions would be based on an average QTc value of triplicate ECGs. If an ECG demonstrated a prolonged QT interval, 2 more ECGs would be obtained over a brief period and the averaged QTc values of the 3 ECGs used to determine whether the patient should be discontinued from the study.

Procedures for Discontinuation of a Patient from the Study

A patient who prematurely discontinues participation in the study will always be asked about the reason(s) for discontinuation and the presence of any AEs. If a patient withdraws consent, the Investigator must ask the patient if he/she is willing, as soon as possible, to be assessed according to the procedures scheduled for the final follow-up visit (Visit 9). If the patient withdrew consent, was non-compliant, or was lost to follow-up, no further follow-up will take place after the final clinical evaluation. The Sponsor will determine on a case-by-case basis if a patient who withdraws for other reasons requires follow-up after the final clinical evaluation. Any ongoing AEs and pregnancies would be followed.

The primary reason for discontinuation/early withdrawal must be specified in the eCRF and final drug accountability must be performed.

### Methodology

Patients will report to the study clinic for a screening visit (Visit 1), followed by a baseline visit on Day 1 (Visit 2) where patients would be randomized and begin treatment. Two remote safety visits over telephone (Days 7 and 49 [Visit 3 and Visit 8]) would be conducted. Once treatment has commenced, there would be 2 in-person safety visits to the clinic (Days 14 and 21 [Visit 4 and Visit 5]), 2 in-person efficacy visits to the clinic (Days 28 and 42 [Visit 6 and Visit 7]) and one follow-up in-person final safety visit (Day 70 [Visit 9]). In total, patients will report to the clinic for 7 in-person visits.

Patients entering the study were randomized in a 2:1 ratio to receive either NLX-112 or placebo. The randomization list was generated by the Clinical Research Organization (CRO), Clinical Trial Consultants (CTC) AB, Uppsala, Sweden. The study utilized competitive enrollment between sites. The original randomization list was kept in a sealed envelope at the CRO and a copy at the hospital pharmacy. Sealed treatment code envelopes were kept by each site. The code envelope was broken when unblinding a study participant who experienced several severe adverse events (SAEs) during the study. The individual doses were dispensed to the patients at the clinics as per the randomization list. Patients self-administered the IMP at home. At each visit to the clinic, patients were asked to return any unused IMP and all empty/partially used IMP containers.

At baseline, symptoms of parkinsonism, depression, anxiety and vital signs were assessed. Electrocardiogramand blood draws for haematology and clinical chemistry were also performed, as well as screening for significant LIDs conducted by a suprathreshold challenge dose of L-DOPA (calculated as 150% of patient's regular dose up to a maximum of 250 mg). L-DOPA was given as Sinemet® (L-DOPA combined with carbidopa in a fixed ratio of 4:1). Patients (and/or caregivers) were required to demonstrate the ability to complete an electronic PD Home Dyskinesia Diary (Kinesia 360 system based on (Hauser et al 2004)), with concordance in ON time with dyskinesia between study staff and patient. Two consecutive 24-hour diaries were completed.

At Visits 2, 6 and 7, efficacy assessments started 30 minutes after the patient had taken 150% of his or her regular L-DOPA dose, when the patient is ON and experiencing typical dyskinesia.

A PD Home Dyskinesia Diary (electronic) would be completed by the patients and/or caregiver with concordance in ON time with dyskinesia between study staff and patient. Two consecutive 24-hour diaries would be completed prior to randomization (baseline, Visit 2) and prior to the clinic visits on Days 28 and 42 (Visits 6 and 7).

A wearable dyskinesia assessment device would be used to monitor dyskinesias during a 2-day period prior to the baseline visit (Day 1, Visit 2) and a 2-day period prior to the clinic visits on Days 28 and 42 (Visits 6 and 7, respectively).

Blood was collected for NLX-112 plasma concentration measurements on Days 14, 21, 28, 42 and 70 (Visits 4, 5, 6, 7 and 9).

Patients self-administered NLX-112 or placebo 2 times each day, once in the morning and once in the evening. Tablets were taken with approximately 240 mL water during a meal. Active tablets contained 0.25 mg NLX-112 and were prepared as described in Example 6 (below). Dose escalation was as follows:

### Up-Titration Period (4 Weeks):

Days 1-4 (0.25 mg/day): 1 tablet in the morning, none in the evening
Days 5-8 (0.5 mg/day): 1 tablet in the morning and 1 tablet in the evening
Days 9-12 (0.75 mg/day): 2 tablets in the morning, 1 tablet in the evening
Days 13-16 (1.0 mg/day): 2 tablets in the morning, 2 tablets in the evening
Days 17-20 (1.25 mg/day): 3 tablets in the morning, 2 tablets in the evening
Days 21-24 (1.5 mg/day): 3 tablets in the morning, 3 tablets in the evening
Days 25-28 (1.75 mg/day): 4 tablets in the morning, 3 tablets in the evening

### Constant Dose Period (2 Weeks):

Days 29-42 (2 mg/day): 4 tablets in the morning, 4 tablets in the evening
   Down-Titration Period (2 Weeks, Down-Titration by 0.25 mg/Day Every 2 Days):
Days 43-44 (start of down-titration period [1.75 mg/day])
Days 45-46 (1.5 mg/day)
Days 47-48 (1.25 mg/day)
Days 49-50 (1 mg/day)
Days 51-52 (0.75 mg/day)
Days 53-54 (0.5 mg/day)
Days 55-56 (0.25 mg/day)

Patients in the placebo arm followed the same dose escalation, constant dose and down-titration schedule, but would be taking the matching placebo tablets. Patients who have intolerable adverse events (AEs) during the 28-day up-titration period would be allowed to return to the previous tolerated dose at the discretion of the Investigator, or they may withdraw from the study if they so choose. A slow down-titration was used to reduce potential withdrawal symptoms.

### Investigational Medicinal Product (IMP), Dosage and Mode of Administration

NLX-112, also known as F13640 and Befiradol, in tablets of 0.25 mg (see Example 6, below) administered orally. NLX-112 was up-titrated to 2 mg/day (or to the highest well-tolerated dose less than 2 mg/day) over 4 weeks, maintained at that dose for 2 weeks, then followed by a 2 week down-titration period.

Placebo was matching tablets (identical weight, shape and color) administered orally using the same dose escalation, constant dose and down-titration schedule.

### Duration of Treatment

Patients were treated for a total of 8 weeks (56 days), receiving the IMP twice daily, once in the morning and once in the evening.

### Duration of Each Patient's Involvement in the Study

The total duration of participation for each patient would be approximately 10 weeks, not including screening.

### Safety Assessments

Safety/tolerability assessments include: medical history (screening), physical examination (screening and follow-up), 12-lead ECG (all clinic visits), vital signs (all clinic visits), AEs (from baseline visit through follow up visit), assessment of suicidal ideation/behavior (C-SSRS; all clinic visits), and clinical labs (hematology, blood chemistry, and urinalysis; all clinic visits).

### Adverse Events

An adverse event (AE) is defined as any untoward medical occurrence in a patient administered a medicinal product and which does not necessarily have a causal relationship with this treatment. An AE can therefore be any unfavorable and unintended sign (including an abnormal laboratory finding), symptom, or disease temporally associated with the use of a medicinal product, whether or not considered related to the medicinal product.

Worsening of dyskinesias (except those associated with the 150% L-DOPA challenge) or PD symptoms compared to baseline would be reported as AEs.

A serious adverse event (SAE) is any AE which:
results in death,
is life-threatening (this refers to a reaction in which the patient was at risk of death at the time of the reaction; it does not refer to a reaction that hypothetically might have led to death if the reaction was more severe),
requires in-patient hospitalization or prolongation of existing hospitalization,
results in persistent or significant disability/incapacity,
is a congenital anomaly/birth defect,
is an important medical event (IME) (this refers to a reaction that may not be immediately life-threatening or result in death or hospitalization, but may jeopardize the patient or may require intervention to prevent any of the other outcomes defined above).

Examples of IMEs are intensive treatment in an emergency room for allergic bronchospasm or blood dyscrasias, convulsions that do not result in hospitalization, development of drug dependency, and drug abuse.

Planned hospitalizations or surgical interventions for a condition that existed before the patient signed the ICF and that did not change in intensity are not SAEs.

If there is any doubt as to whether an AE meets the definition of an SAE, a conservative viewpoint must be taken, and the AE must be reported as an SAE.

The term adverse drug reaction (ADR) is to be used whenever either the Investigator or Sponsor or designee assessed the AE as at least possibly related to the IMP.

The term Serious Adverse Drug Reaction (SADR) is to be used whenever either the Investigator or Sponsor or designee assessed the SAE as at least possibly related to the IMP.

All AEs (including SAEs) would be collected from the start of IMP administration until the end-of-study visit.

Any AE with start date on the day of IMP administration will, if possible, be recorded with start time.

At the end-of-study visit, information on new AEs or SAEs, if any, and stop dates for ongoing events must be recorded as applicable.

Investigators are not obligated to actively seek AEs or SAEs after conclusion of the study participation. However, if the Investigator learns of any SAE, including a death, at any time after a participant has been discharged from the study, and he/she considers the event to be reasonably related to the study intervention or study participation, the Investigator must promptly notify the Sponsor.

The grading of the intensity of AEs follow the common terminology criteria for AEs (CTCAE) v5.0. Grade refers to the severity of the AE. The CTCAE displays Grades 1 through 5 with unique clinical descriptions of severity for each AE based on this general guideline.

The Investigator must assess the intensity of an AE using the following definitions, and record it on the AE Log in the eCRF:
Grade 1 Mild; asymptomatic or mild symptoms; clinical or diagnostic observations only; intervention not indicated.
Grade 2 Moderate; minimal, local or non-invasive intervention indicated; limiting age-appropriate instrumental ADL*.
Grade 3 Severe or medically significant but not immediately life-threatening; hospitalization or prolongation of hospitalization indicated; disabling; limiting self-care ADL**.
Grade 4 Life-threatening consequences; urgent intervention indicated.
Grade 5 Death related to AE. *Instrumental ADL refer to preparing meals, shopping for groceries or clothes, using the telephone, managing money, etc.**Self-care ADL refer to bathing, dressing and undressing, feeding self, using the toilet, taking medications, and not bedridden.

The Investigator must assess the causal relationship between an AE and the IMP using the definitions below and record it the AE Log of the eCRF:
Probable The event has a strong temporal relationship to the IMP or recurs on re-challenge, and another etiology is unlikely or significantly less likely.
Possible The event has a suggestive temporal relationship to the IMP, and an alternative etiology is equally or less likely.
Unlikely The event has no temporal relationship to the IMP or is due to underlying/concurrent illness or effect of another drug (that is, there is no causal relationship between the IMP and the event).

An AE is considered causally related to the use of the IMP when the causality assessment is probable or possible.

The Investigator must assess the outcome of an AE using the definitions below and record it on the AE Log of the eCRF:
Recovered/resolved The patient has recovered completely, and no symptoms remain.
Recovering/resolving The patient's condition is improving, but symptoms still remain.
Recovered/resolved The patient has recovered, but some symptoms remain (for
with sequelae example, the patient had a stroke and is functioning normally but has some motor impairment).
Not recovered/not The patient's condition has not improved and the symptoms are resolved unchanged (for example, an atrial fibrillation has become chronic).
Fatal
Unknown

The Investigator must report the action taken with study treatment using the definitions below and record it on the AE Log of the eCRF:
Dose increased
Dose not changed
Dose rate reduced
Dose reduced
Drug interrupted
Drug withdrawn
Not applicable
Unknown

AEs identified using any of the following methods would be recorded:
AEs spontaneously reported by the patient.
AEs observed by the Investigator or medical personnel.
AEs elicited based on non-leading questions from the Investigator or medical personnel.

AEs must be recorded in the AE Log of the eCRF. The Investigator must provide information on the AE, preferably with a diagnosis or at least with signs and symptoms; start and stop dates, start and stop time; intensity; causal relationship to IMP; action taken, and outcome.

If the AE is serious, this must be indicated in the eCRF.

AEs, including out-of-range clinically significant clinical safety laboratory values, must be recorded individually, except when considered manifestations of the same medical condition or disease state; in such cases, they must be recorded under a single diagnosis.

### Physical Examination

The physical examination should include examination of the following: general appearance, head, ears, eyes, nose, throat, neck, skin, cardiovascular system, respiratory system, abdominal system, and nervous system.

Any abnormalities would be specified and documented as clinically significant (CS) or not clinically significant (NCS). Abnormal post-dose findings assessed by the Investigator as clinically significant would be reported as AEs.

### Vital Signs

Blood pressure (systolic and diastolic) and pulse would be measured in sitting position after at least 5 minutes of rest and then again 3 minutes after standing upright to determine whether the patient has orthostatic hypotension (decrease in systolic blood pressure of at least 20 mm Hg or diastolic blood pressure of at least 10 mm Hg within 3 minutes of the patient standing up, compared to the blood pressure obtained while in the sitting position). At screening and baseline (Visit 1 and Visit 2), the investigator should collect 3 assessments, each taken 15 to 20 minutes apart, in order to obtain an average value. All measurements and the calculated average value should be recorded in the eCRF. At other visits, one assessment is sufficient.

Respiratory rate would be assessed by routine methods. Body temperature would be measured orally using a digital thermometer.

Any vital signs outside the normal ranges at each site would be judged as NCS or CS. The assessment would be recorded in the eCRF. Post-dose vital signs judged as “abnormal, clinically significant” by the Investigator would be reported as AEs.

### Electrocardiogram

Single 12-lead ECG would be recorded in supine position after at least 5 minutes of rest using an ECG machine. The following parameters would be recorded: rhythm, ventricular rate, PR interval, QRS duration, QT, QTcB, and QTcF. In case of an abnormal ECG, the ECG would be repeated once as judged by the Investigator. In case of a QTcF ≥500 ms, refer to the withdrawal criteria.

Any abnormalities would be specified and documented as clinically significant or not clinically significant. Abnormal post-dose findings assessed by the Investigator as clinically significant would be reported as AEs.

### Columbia Suicide Severity Rating Scale (C-SSRS)

The C-SSRS is an assessment tool designed to quantify the severity of suicidal ideation and behavior as rated by the investigator. The baseline scale would be used at screening (Visit) and the follow-up scale at all subsequent visits.

The scale contains 6 “yes” or “no” questions in which respondents are asked to indicate whether they have experienced several thoughts or feelings relating to suicide over the past month and behavior over their lifetime and past 3 months. Each question addresses a different component of the respondent's suicide ideation severity and behavior.
Question 1: wish to be dead
Question 2: non-specific suicidal thoughts
Questions 3-5: more specific suicidal thoughts and intent to act
Question 6: suicidal behavior over the respondent's lifetime and past 3 months
If the respondent answers “yes” to Question 2, he/she is instructed to answer Questions 3-5. If the respondent answers “no” to Question 2, he/she may skip to Question 6.

An answer of “yes” to any of the 6 questions may indicate a need for referral to a trained mental health professional and an answer of “yes” to questions 4, 5 or 6 indicate high-risk.

### Laboratory Safety Assessments

Blood samples for analysis of clinical chemistry, hematology and coagulation parameters would be collected through venipuncture or an indwelling venous catheter and analyzed by routine analytical methods by local hospital laboratories. Urine analysis would be performed at the research clinic using dip sticks. Pregnancy test (urine) would be performed at the research clinic at screening.

Any lab values outside the normal ranges would be judged as NCS or CS. The assessment would be recorded in the eCRF. Abnormal values assessed by the Investigator as clinically significant would be reported as AEs. If an abnormal value is associated with corresponding clinical signs or symptoms, the sign/symptom should be reported as the AE.

TABLE 1
Safety Laboratory Parameters

| Category | Parameter |
|---|---|
| Clinical | Alanine aminotransferase (ALT) |
| chemistry | Albumin |
| | Alkaline phosphatase (ALP) |
| | Aspartate aminotransferase (AST) |
| | Bilirubin (total and conjugated) |
| | Calcium |
| | Chloride |
| | C-reactive protein (CRP) (screening only) |
| | Creatine phosphokinase (CPK) |
| | Creatinine (eGFR included) |
| | Gamma glutamyl transpeptidase (GGT) |
| | Glucose |
| | Phosphate |
| | Potassium |
| | Sodium |
| | Urea (nitrogen) |
| | Uric acid |
| Hematology | Hematocrit |
| | Hemoglobin (Hb) |
| | Platelet count |
| | Red blood cell (RBC) count |
| | White blood cell (WBC) count with differential count |
| | Mean corpuscular volume (MCV) |
| | Mean corpuscular hemoglobin (MCH) |
| | Mean corpuscular hemoglobin concentration (MCHC) |
| Coagulation | Activated Partial Thromboplastin Time (APTT) |
| | Prothrombin Complex International Normalized Ratio |
| | (PK[INR]) |
| Urinalysis | Bilirubin |
| (dip stick) | Blood |
| | Erythrocytes |
| | Glucose |
| | Ketones |
| | Leucocytes |
| | Nitrites |
| | pH |
| | Albumin |
| Pregnancy | Urine pregnancy test at screening¹ |
| test | |

¹Females of child-bearing potential only

### Efficacy Assessments

The following efficacy assessments were performed:
UDysRS
PD Home Dyskinesia Diary (ON time without troublesome dyskinesia)
UPDRS, parts I-IV
CGI-S, CGI-C
Wearable dyskinesia assessment device data collection
KPPS
PDQ39
HADS
ICIQ-OAB
ESS

Efficacy in treating LID, as well as other non-motor symptoms of PD, would be assessed with the UDysRS, UPDRS, CGI-S, CGI-C, PDQ39, and a PD Home Dyskinesia Diary (electronic). Dyskinesia will also be monitored using a wearable dyskinesia assessment device. In addition, 2 exploratory scales, the KPPS and the ICIQ-OAB will assess potential treatment effects on various types of pain and on overactive bladder, respectively, and one assessment of sleep (the ESS) would be used to evaluate sleep quality, sleep disturbances and sleepiness while engaged in 8 activities.

Efficacy assessments were collected on Days 1, 28 and 42 (Visits 2, 6 and 7, respectively). The efficacy assessments at Visits 2, 6 and 7 should be performed in the order outlined in Table 2.

TABLE 2
Order of Efficacy Assessments

Assessor

| Assessment | Investigator | Patient |
|---|---|---|
| UDysRS (Parts III and IV repeated | X | |
| 3 times at approximately 30, 60 | | |
| and 90 minutes after the 150% L- | | |
| DOPA challenge) | | |
| UPDRS PartIII in ON (within 30-90 | X | |
| minutes after the 150% L-dopa | | |
| challenge) | | |
| UPDRS parts I, II and IV | X | X¹ |
| CGI-C | X | |
| KPPS | X | |
| UDysRS (Parts I and II) | | X |
| PDQ39 | | X |
| HADS | | X |
| ESS | | X |
| ICIQ-OAB | | X |

CGI-C = Clinical Global Impression of Change,
ESS = the Epworth Sleepiness Scale,
HADS = the Hospital Anxiety Depression Scale,
ICIQ-OAB = the International Consultation on Incontinence Questionnaire Overactive Bladder Module,
KPPS = the King’s Parkinson’s disease Pain Scale,
UPDRS = Unified Parkinson’s Disease Rating Scale,
PDQ39 = the Parkinson’s disease Quality of Life Scale (39-item),
UDysRS = the Unified Dyskinesia Rating Scale
¹End of Part I and Part II

Diary data would be based on 2 consecutive 24-hour diaries taken prior to the baseline visit (Visit 2) and prior to Days 28 and 42 (Visits 6 and 7, respectively). Similarly, wearable data collection will take place during a 2-day period prior to the baseline visit (Visit 2) and a 2-day period prior to the clinic visits on Days 28 and 42 (Visits 6 and 7, respectively).

### L-DOPA Challenge

The patient took 150% of their normal L-DOPA dose 30 minutes prior to the start of efficacy assessments (UDysRS) at Day 1 (Visit 2), Day 28 (Visit 6) and Day 42 (Visit 7).

The challenge should be performed approximately 2 to 2.5 hours after the patient's morning dose.

Patients who use continuous intestinal L-DOPA infusion (Duodopa or Lecigon) should instead stop the infusion for 15-30 minutes before the administration of an oral L-DOPA dose equivalent to 300% of their hourly infusion L-DOPA equivalent dose (LED*). The time of the day and the time between stopping the pump and administering the oral dose should be the same on all assessments. (*100 mg Duodopa=111 mg LED, 100 mg Lecigon=148 mg LED)

The maximum L-DOPA dose to be given is 250 mg.

The UDysRS (Parts III and IV) was repeated 3 times after each L-DOPA challenge, 30 minutes apart, and hence performed approximately 30, 60 and 90 minutes after each challenge.

Should the Investigator consider it medically warranted to reduce the L-DOPA challenge in a specific patient for safety or tolerability reasons, this should be approved by the Sponsor prior to implementation. It is critical that the same dose is given at all visits with L-DOPA challenges for a given patient.

### Unified Dyskinesia Rating Scale (UDysRS)

The UDysRS is a rating instrument designed to assess the core features of dyskinesia in Parkinson's Disease. The UDysRS consists of 4 parts:
r. Part I, historical disability with regard to the patient's perceptions of the impact on activities of ADL of on-dyskinesia.
s. Part II, historical disability with regard to the patient's perceptions of the impact on ADL of off-dystonia.
t. Part III, objective impairment, which assesses severity of dyskinesia, affected body parts, and type of impairment (choreic vs. dystonic).
u. Part IV, objective disability, based on an evaluation of Part III activities.

Each item in the UDysRS is scored from 0 to 4, with a possible maximum total score of 104.

The UDysRS assessment would be performed at baseline (Day 1, Visit 2), Day 28 and Day 42 (Visits 6 and 7) starting approximately 30 minutes after the L-DOPA challenge on these days. The UDysRS (Parts III and IV) was repeated 3 times after each L DOPA challenge, 30 minutes apart, and hence performed approximately 30, 60 and 90 minutes after each challenge.

The data for all 3 assessments performed at each visit would be entered in the eCRF and the Investigator will highlight which of the assessments, corresponding to the assessment where the patient had most severe dyskinesia, that would be used in the descriptive summaries of data.

The UDysRS assessments would be videotaped at Visits 2, 6 and 7. The video recordings are planned to be used as a backup to the standard investigator rating; for example, to ensure consistency between assessments in case several assessors are involved, or if other issues arise. It may be possible that all video recordings are assessed by a common central rater.

### PD Home Dyskinesia Diary

A PD Home Dyskinesia Diary (electronic) would be completed by the patients and/or caregiver with concordance in ON time with dyskinesia between study staff and patient. The diary is integrated in the Kinesia 360 wearable dyskinesia assessment system and is based on the PD Home Diary developed by Hauser (Hauser R. A., Deckers F., Lehert P. Parkinson's Disease Home Diary: Further Validation and Implications for Clinical Trials. Mov. Discord. 19 (12), 2004). The diary would be used to score 5 different conditions in 30-minute time intervals during 2×24 hours prior to Visit 2, Visit 6 and Visit 7:
ASLEEP;
OFF;
ON (i.e., adequate control of PD symptoms) without dyskinesia;
ON with non-troublesome dyskinesia;
ON with troublesome dyskinesia.

### Unified Parkinson's Disease Rating Scale (UPDRS)

The UPDRS is one of the most widely used rating scales employed in the assessment of Parkinson's disease. The UPDRS consists of 4 parts:
v. Part I assesses non-motor experiences of daily living, such as cognitive impairment and depressed mood.
w. Part II assesses motor experiences of daily living, such as speech and eating tasks.
x. Part III is a motor examination conducted by the clinician, including assessments of symptoms such as rigidity and tremor.
y. Part IV is an assessment of motor complications, such as time spent with dyskinesia and functional impact of dyskinesias.

Each item in the UPDRS is scored from 0 to 4, and the individual scores are summed to give a total score that indicates the severity of the disease, with a score of 0 indicating no disability and a score of 199 being the most severe (indicating total disability).

### Clinical Global Impression of Severity and Change (CGI-S, CGI-C)

The CGI-C is a clinician-oriented scale that assesses the total improvement in the patient's condition relative to the clinical global impression of severity (CGI-S) scale conducted at baseline. The CGI-C rates the patient's condition from 0 to 7, with a rating of 0 indicating “no assessment”, a rating of 1 indicating “very much better”, and a rating of 7 indicating “very much worse”.

### Wearable Dyskinesia Assessment Device

The Kinesia 360 (Great Lakes Neurotechnologies, Inc) wearable dyskinesia assessment system would be used to monitor dyskinesias. Wearable data collection of dyskinesias will take place during a 2-day period prior to the baseline visit (Visit 2) and a 2-day period prior to the clinic visits on Days 28 and 42 (Visits 6 and 7, respectively).

The Kinesia 360 motor assessment system provides low burden method for remote, continuous measurement of patient symptoms. Sensors worn on the wrist and ankle combined with a mobile application continuously record data for assessment of tremor, slowness, dyskinesia and mobility. Data from the motion sensors is uploaded to the Kinesia Web Portal and algorithms are used to detect symptoms and calculate severity scores every 2 minutes on a scale shown to be highly correlated with clinician ratings. Sensors record data all day and recharge overnight for extended home use.

Each patient would be trained and provided with a user guide in Swedish, sensors and a smartphone that is preloaded with the Kinesia 360 software app. The app guides patients through daily use and includes electronic diaries for capturing patient-reported outcomes and a medication diary. The system also includes mobile device management to ensure that the software is always current.

Motion and diary data are transmitted to a secure cloud via mobile broadband for processing and storage in a 21 CFR Part 11 compliant database. Clinicians and researchers can access reports via the Kinesia web portal and view real-time patient compliance.

### Assessments Related to Exploratory Endpoints

### King's Parkinson's Disease Pain Scale (KKPS)

The KPPS is a rater-interview-based scale conducted with the patient and caregiver (if available). It is a 14-item scale covering 7 domains of pain:
z. Musculoskeletal pain
aa. Chronic pain
bb. Fluctuation-related pain
cc. Nocturnal pain
dd. Orofacial pain
ee. Edema/swelling; discoloration
ff. Radicular pain

Each item is scored by severity (0-3; none to very severe) multiplied by frequency (0-4; never to all the time), for an item sub score of 0-12, resulting in a total score from 0 to 168. Ratings are based on the previous month, or in the case of this study, the intervening period since the previous clinic visit (about 3 weeks). Treatment effects would be analyzed as change from baseline for total score and for individual pain domains.

### Parkinson's Disease Questionnaire (PDQ39)

The PDQ39-item patient reported outcome of health status and quality of life and is the most frequently used disease-specific health status measure for PD. The questionnaire assesses how often people affected by PD experience difficulties across 8 dimensions of daily living. The standard PDQ39 assesses quality of life over the previous month. In this study the initial time period assessed at the Baseline Visit would be the previous month, with subsequent assessments for the intervening period since the last clinic visit (about 3 weeks).

The 8 quality of life dimensions covered in the questionnaire are:
gg. Mobility (10 items #1-10)
hh. ADL (6 items #11-16)
ii. Emotional Well Being (6 items #17-22)
jj. Stigma (4 items #23-26)
kk. Social Support (3 items #27-29)
ll. Cognition (4 items #30-33)
mm. Communications (3 items #34-36)
nn. Bodily Discomfort (3 items #37-39)

Individual items are rated by the patient on a scale of 0 to 5. Dimension scores are calculated on a scale of 0 to 100. The total score and individual dimension scores would be assessed for change from baseline.

### Hospital Anxiety Depression Scale (HADS)

The HADS is used to determine the levels of anxiety and depression that a person is experiencing. The questionnaire consists of 7 items related to anxiety and 7 related to depression. Each item on the questionnaires is scored from 0-3, which means that a person can score between 0 and 21 for either anxiety or depression.

### International Consultation on Incontinence Questionnaire Overactive Bladder Module (ICIQ-OAB)

The ICIQ-OAB provides a brief and robust measure to assess the impact of symptoms of overactive bladder on quality of life and outcome of treatment. It is a 4-item patient reported outcome covering 4 domains of bladder function: frequency, nocturia, urgency, and urge urinary incontinence. Each item is scored by frequency of 0-4, for a total score of 0-16. Ratings would be based on the previous month or since the last clinic visit. Efficacy would be assessed as change from the baseline total score.

### Epworth Sleepiness Scale (ESS)

The ESS is a self-administered questionnaire with 8 questions. Respondents are asked to rate, on a 4-point scale (0-3), their usual chances of dozing off or falling asleep while engaged in 8 different activities. The ESS score (the sum of 8 item scores, 0-3) can range from 0 to 24. Ratings are based on the previous month, or in the case of this study, the intervening period since the previous clinic visit (about 3 weeks).

### Rater and Self-Assessments

An independent neurologist or the treating physician will rate patients at each clinical site. At each applicable visit (Visits 2, 6 and 7) the same rater (if possible) will conduct efficacy assessments beginning 30 minutes after the patient has taken 150% of his or her regular L-DOPA dose, when the patient is ON and experiencing typical dyskinesia. The UDysRS assessments would be videotaped at Visits 2, 6 and 7.

Patients (and/or caregivers) need to demonstrate the ability to complete the electronic PD Home Dyskinesia Diary, with concordance in ON time with dyskinesia between study staff and patient.

A wearable dyskinesia assessment device would be used to monitor dyskinesias during a 2-day period prior to the baseline visit (Day 1, Visit 2) and a 2-day period prior to the clinic visits on Days 28 and 42 (Visits 6 and 7, respectively).

### Statistical Methods and Determination of Sample Size

### General

Continuous data would be presented in terms of evaluable and missing observations, arithmetic mean, standard deviation (SD), median, minimum and maximum value.

Categorical data would be presented as counts and percentages. When applicable, summary data would be presented by treatment, and by assessment time. Individual patient data would be listed by patient number, treatment, and, where applicable, by assessment time.

All descriptive summaries and statistical analyses would be performed using SAS Version 9.4 or later (SAS Institute, Inc., Cary, NC).

Baseline would be defined as the last non-missing data point prior to the first administration of IMP.

The primary objective of this study is to evaluate the safety and tolerability of NLX-112. The secondary objective is to evaluate preliminary efficacy endpoints, hence, statistical tests of efficacy outcomes would be for descriptive purposes and no adjustment for multiple tests would be performed.

No imputation of missing data would be performed.

All data would be listed by treatment and patient.

### Determination of Sample Size

No formal sample size calculation has been performed. A sample size of 24 patients is considered sufficient to evaluate the primary objective and to allow for a preliminary efficacy evaluation.

### Analysis Populations

The safety analysis set will comprise all patients who received at least one dose of the IMP.

The full analysis set (FAS) will comprise all randomized patients who were dosed and who provided at least one post-baseline measurement at any timepoint and for any data type.

The per protocol set (PPS) will consist of all patients included in the FAS population who do not have any major protocol violations assessed to compromise the analysis of study data. All protocol violations would be judged as major or minor prior to database lock.

### Description of Study Population

Descriptive statistics for demographics, weight and height would be presented by treatment.

Medical/psychiatric history would be presented by system organ class (SOC) and preferred term (PT) by treatment. Prior/concomitant medications would be presented by ATC level 1, 3 and 5 by treatment.

The number of patients treated in each treatment period and their individual dose would be listed. Compliance would be presented using summary statistics per treatment (1-[number of study drugs returned/number of study drug delivered]).

### Analysis of Primary Endpoints

The safety analyses would be based on the safety dataset.

An overview of all AEs, including SAEs, intensity, relationship to IMP, and deaths would be presented by treatment. Incidence of AEs and SAEs would be summarized by SOC and PT by treatment. Separate tables would be provided, if relevant, for serious AEs (SAEs) or events leading to withdrawal from study. All AE data would be listed by patient and include the verbatim term entered by the Investigator.

Clinically significant and non-clinically significant abnormal findings would be specified and presented by patient and summarized by treatment. Changes over time would be presented using shift tables, if considered appropriate.

Vital signs (systolic/diastolic blood pressure, orthostatic blood pressure, pulse, respiratory rate and body temperature) would be summarized by treatment. Data would be presented with absolute and percent change from baseline.

All ECGs would be categorized as “normal,” “abnormal, not clinically significant,” or “abnormal, clinically significant” (as judged by the Investigator) and summarized by treatment using frequency tables. Changes over time would be presented using shift tables, if considered appropriate.

C-SSRS data would be listed by patient and visit and summarized by treatment. Changes over time would be presented using shift tables, if considered appropriate.

Safety laboratory data would be summarized by treatment with absolute and percent change from baseline at each visit. Abnormal, clinically significant values would be summarized separately, if considered appropriate.

### Analysis of Secondary Endpoints

The efficacy analyses would be performed separately on both the FAS and PPS if deemed appropriate.

Change from baseline in the UDysRS total score to Visit 6 (Day 28) and Visit 7 (Day 42) respectively would be summarized using descriptive statistics. A Wilcoxon signed-rank test would be used to assess statistically significant differences between active treatment and placebo for each time point. The same analysis would be done for UDysRS total objective score Part III and IV only.

Change from baseline in ON Time Without Troublesome Dyskinesia (ON Without Dyskinesia plus ON With Non-troublesome Dyskinesia) based on a PD Home Dyskinesia Diary would be summarized using descriptive statistics for all assessment time points. A Chi-Square test would be used to assess statistically significant differences between active treatment and placebo for the whole study period.

Change from baseline in UPDRS combined scores (Parts I, II, III and IV) to Visit 6 (Day 28) and Visit 7 (Day 42) respectively would be summarized using descriptive statistics. A Wilcoxon signed-rank test would be used to assess statistically significant differences between active treatment and placebo for each time point. The same analysis would be done for UPDRS Part III only.

Change from baseline in CGI-C in overall PD symptoms to Visit 6 (Day 28) and Visit 7 (Day 42) respectively would be summarized using descriptive statistics. A Wilcoxon signed-rank test would be used to assess statistically significant differences between active treatment and placebo for each time point.

Change from baseline in dyskinesia scores measured by the Kinesia 360 (Great Lakes Neurotechnologies, Inc) wearable dyskinesia assessment system to Visit 6 (Day 28) and Visit 7 (Day 42) respectively would be summarized using descriptive statistics. A Wilcoxon signed-rank test would be used to assess statistically significant differences between active treatment and placebo for each time point.

Hypotheses tests were performed at the 5% alpha level with the aim of detecting superiority of NLX-112 compared to placebo on the change from baseline on a number of efficacy parameters. Any inference of superiority of NLX-112 compared to placebo was to be based on these tests. Further testing within each treatment (NLX-112 or placebo respectively) at the 5% alpha level was done with the aim of detecting a beneficial shift from baseline, as a result of the IMP intervention. Statistical tests of efficacy outcomes were for explorative purposes and no adjustment for multiple tests was performed.

No formal sample size calculation had been performed. A sample size of 24 patients was considered sufficient to evaluate the primary objective and to allow for a preliminary efficacy evaluation. Data are presented in terms of arithmetic mean and standard deviation (SD). When applicable, summary data are presented by treatment, and by assessment time.

Data analyzers were blinded until after the database was locked.

### Analysis of Exploratory Endpoints

Change from baseline in the KPPS total score to Visit 6 (Day 28) and Visit 7 (Day 42) respectively would be summarized using descriptive statistics. A Wilcoxon signed-rank test would be used to assess statistically significant differences between active treatment and placebo for each time point.

Change from baseline in the PDQ39 total score to Visit 6 (Day 28) and Visit 7 (Day 42) respectively would be summarized using descriptive statistics. A Wilcoxon signed-rank test would be used to assess statistically significant differences between active treatment and placebo for each time point.

Change from baseline in the HADS to Visit 6 (Day 28) and Visit 7 (Day 42) respectively would be summarized using descriptive statistics. A Wilcoxon signed-rank test would be used to assess statistically significant differences between active treatment and placebo for each time point.

Change from baseline in the ICIQ-OAB total score to Visit 6 (Day 28) and Visit 7 (Day 42) respectively would be summarized using descriptive statistics. A Wilcoxon signed-rank test would be used to assess statistically significant differences between active treatment and placebo for each time point.

Change from baseline in the ESS to Visit 6 (Day 28) and Visit 7 (Day 42) respectively would be summarized using descriptive statistics. A Wilcoxon signed-rank test would be used to assess statistically significant differences between active treatment and placebo for each time point.

### Rater and Self-Assessments

An independent neurologist or the treating physician will rate patients at each clinical site. At each applicable visit (Visits 2, 6 and 7) the same rater (if possible) will conduct efficacy assessments beginning 30 minutes after the patient has taken 150% of his or her regular L-DOPA dose, when the patient is ON and experiencing typical dyskinesia. The UDysRS assessments would be videotaped at Visits 2, 6 and 7.

### Results

35 patients were screened, 27 patients were randomized (18 to NLX-112, 9 to placebo). 3 NLX-112 patients discontinued (withdrawal of consent) and 15 completed. 1 placebo patient discontinued (DBS surgery) and 8 completed. 23 subjects completed the study.

The study was conducted during the Covid-19 pandemic. The first patient was screened on 9 Nov. 2021, the first patient was randomized and dosed on 24 Nov. 2021 and the last patient completed the last visit on 18 Jan. 2023. One patient in the placebo group was diagnosed with Covid-19 during the down-titration period but remained in the study. For a few patients, intended visits were postponed due to Covid-19-like symptoms, which in some cases resulted in the down-titration period starting prior to Visit 7 (Day 42±2 days). A continuous risk assessment, comprising assessment of potential risks associated with Covid-19, was performed and mitigating actions were implemented accordingly to preserve patient safety and data quality/integrity in accordance with EMA guidelines as well as guidelines and restrictions from local authorities.

Four patients dropped-out during the up-titration period, 3 on NLX-112 (all due to withdrawal of consent) and 1 on placebo (due to deep brain stimulation operation). All drop-outs were included in the Full Analysis Set (FAS) but were excluded from the Per Protocol Set (PPS). One additional placebo-patient was excluded from the PPS because of a protocol violation (use of gabapentin, prohibited medication) prior to unblinding. Evaluation of safety and tolerability was based on the FAS (N=27) while efficacy evaluation was based on the PPS (N=22). The baseline characteristics of enrolled patients are summarized in Table 3. Information regarding the highest dose achieved by each patient is provided in Table 4.

TABLE 3
Patient Baseline Characteristics

| Assessment | Unit | NLX-112 (N = 18) | | Placebo (N = 9) | |
|---|---|---|---|---|---|
| Age | Years, mean (SD) | 65.7 | (9.7) | 64.6 | (6.3) |
| Sex | ‘n’ (male %) | 10/18 | (56%) | 5/9 | (56%) |
| Ethnicity | Hispanic or Latino | 1/18 | (5.6%) | 0 | |
| | Not Hispanic or Latino | 17/18 | (94%) | 9/9 | (100%) |
| Race | ‘n’ (white %) | 17/18 | (94%) | 9/9 | (100%) |
| Time since PD diagnosis | Years, mean (SD) | 11.4 | (4.2) | 9.9 | (4.6) |
| Hoehn & Yahr score | Mean (SD) | 2.41 | (0.65) | 2.39 | (0.39) |
| MMSE score | Mean (SD) | 29.3 | (0.9) | 29.1 | (1.1) |
| LEDD^{a} | Mean (SD) | 1128 | (445) | 1759 | (1095) |
| UPDRS total | Mean (SD) | 38.3 | (14.9) | 42.4 | (13.2) |
| UPDRS Part3 | Mean (SD) | 17.8 | (10.3) | 16.9 | (9.0) |
| UDysRS total | Mean (SD) | 31.8 | (8.9) | 43.7 | (14.8) |
| (Pt3 max)^{b} | | | | | |
| UDysRS total | Mean (SD) | 30.4 | (9.6) | 42.7 | (14.7) |
| (Pt3 average)^{c} | | | | | |

^{a}L-DOPA Equivalent Daily Dose (Jost S T et al., “Levodopa dose equivalency in Parkinson’s disease: updated systematic review and proposals,” Movement Disorders, 2023, 38, 1236-1252).
^{b}UDysRS total score, with Part 3 based on the highest score of the 3 assessment sessions (30, 60, 90 min after L-DOPA dose).
^{c}UDysRS total score, with Part 3 based on the average score of the 3 assessment sessions (30, 60, 90 min after L-DOPA dose).

TABLE 4
Patient Highest Titration Dose

| | |
|---|---|
| NLX-112 (n = 15) | Placebo (n = 8) |

| Dose (mg) | n | % | n | % |
|---|---|---|---|---|
| 2.0 | 12 | 80 | 5 | 62.5 |
| 1.75 | 2 | 13.3 | — | — |
| 1.5 | — | — | 1 | 12.5 |
| 1.25 | — | — | 1 | 12.5 |
| 1.0 | 1 | 6.7 | 1 | 12.5 |
| 0.75 | — | — | — | — |
| 0.5 | — | — | — | — |
| 0.25 | — | — | — | — |

### Determination of NLX-112 in Human Plasma Using UPLC-MS/MS

The concentration of NLX-112 was determined in 120 human plasma samples collected from the clinical study. The bioanalysis was performed in the calibration range of 1.0 ng/ml to 1620 ng/ml using a 35 μL sample volume.

The Qualification Runs 1 and 2 contained two calibration curves, six sets of QC samples at eight levels (QC1 to QC8), as well as UPLC and Tecan Carry-over test samples. The analytical run 1 contained two calibration curves, four sets of QC samples at three levels (QC L, M and H), as well as UP>C and Tecan Carry-over test samples. The qualification and analysis was performed in an exploratory setting.

The analytical instrumentation consisted of an Acquity UPLC system coupled to a Xevo-TQS micro tandem quadrupole mass spectrometer (Waters Corp., Milford, MA, USA). The ionization technique used was electrospray ionization (ESI) in positive mode.

A combined calibration curve was constructed from the two sets of calibration samples for each run. A quadratic calibration model (y=ax²+bx+c) with a weight of 1/x was used. The established calibration curve from each run was used for the calculation of the QC, and study sample concentrations. The lower limit of quantification (LLOQ) for the method was 1.0 ng/mL and the upper limit of quantification (ULOQ) was 1620 ng/mL of NLX-112 in human plasma.

The results and evaluation of the method performance show that the bioanalytical method for the determination of NLX-112 in human plasma in the range 1.0 to 1620 ng/ml is sufficiently sensitive, robust, and accurate for the determination of NLX-112 in human plasma samples.

The mean NLX-112 blood plasma concentration measured in treated patients on study days 14 (Visit 4), 25 (Visit 5), 28 (Visit 6), 42 (Visit 7), and 70 (Visit 9) is shown in Table 5. SEM=standard error of the mean.

TABLE 5
NLX-112 Blood Plasma Concentrations

| | Visit 4 | Visit 5 | Visit 6 | Visit 7 | Visit 9 |
|---|---|---|---|---|---|
| Mean (ng/mL) | 32.3 | 38.7 | 71.0 | 72.0 | 3.9 |
| SEM (ng/mL) | 4.3 | 4.6 | 9.9 | 13.2 | 3.9 |

### Safety/Tolerability

Adverse events observed during the clinical study are summarized in Tables 6-9. In each Table, the data based on the full analysis set; n=number of subjects, m=number of events; percentages are based on the number of subjects receiving respective treatment; and the following AEs are coded with multiple MedDRA terms and are represented as separate AEs in tables and listings: ‘Pain, left elbow due to fall’, ‘Redness of eyelid and swelling of eyelid’, ‘Worsening of Restless legs symtom’. In Table 8, NU=NLX-112 up-titration; NS=NLX-112 steady state; ND=NLX-112 down-titration; PU=Placebo up-titration; PS=Placebo steady state; PD=Placebo down-titration; FU=Follow-up; T=Total; CA or BD=Congenital Anomaly or Birth Defect; PSD/I=Persistent or Significant Disability/Incapacity; RPH=Requires or Prolongs Hospitalization; ILT=Is Life Threatening; OMISE=Other Medically Important Serious Event; WFS=withdrawal from study.

Twenty-three (85%) of the 27 randomized patients, 16 (89%) on NLX-112 and 7 (78%) on placebo, reported 103 AEs over the course of the study. Overall, there was no obvious difference in AE-reporting frequency, intensity or causality assessments between patients on NLX-112 and patients on placebo (Tables 6-9). AEs were more frequently reported during the up-titration period in both treatment groups (Tables 8, 9). The safety data collected during the study indicates that the treatment of Parkinson's disease patients with NLX-112 is safe and well tolerated at the blood plasma concentrations recorded in Table 5. Concerning laboratory assessments, there were no clinically significant changes in any clinical chemistry, hematology, urinalysis, or coagulation parameters. Concerning ECG assessments, there were no changes in HR, PR, QRS, QT, or QTc measures at any post-baseline visit. Concerning vital signs, there were no changes in HR, BP, body temperature, etc. There were no serious adverse events in the NLX-112 group. One SAE (syncope, severe, possibly related) occurred in 1 patient treated with placebo. One patient treated with NLX-112 withdrew consent due to AEs (on and off phenomenon, vomiting and stress, possibly related) and prematurely discontinued the study. Approximately two thirds of the AEs (66 out of 103) were assessed as possibly (m=53) or probably (m=13) related to treatment while the remaining third (m=37) were assessed as unlikely related to treatment. Most AEs (100 out of 103) were assessed as mild (m=67) or moderate (m=33) in intensity. The most common AEs reported by patients on NLX-112 were nausea, parkinsonism and headache. Insomnia, vomiting, fatigue, orthostatic hypotension, dizziness, vertigo, fall, back pain dissociation and restless legs syndrome were all reported by 2 patients (11%) on NLX-112. Headache, parkinsonism, vomiting, fatigue, orthostatic hypotension, dizziness and vertigo also occurred in the placebo-group. There were no clinically significant changes from baseline in overall ECG evaluations or parameters, mean safety laboratory parameters, and physical examination findings and no obvious differences between the active treatment group and the placebo group in the change from baseline of these variables. Isolated abnormal parameters were assessed as clinically significant in both treatment groups but there were no trends associated with any safety variable in either treatment group. Eighteen (67%) of the 27 randomized patients, 12 (67%) on NLX-112 and 6 (67%) on placebo, had abnormal vital signs assessed as not clinically significant on one or more occasions during the study. Of these, 2 patients, both on NLX-112, had abnormal vital signs assessed as clinically significant: one patient had systolic hypertension (mild, unlikely related). The patient had high systolic blood pressure also at screening (sitting and standing) and increased respiratory rate at the follow-up visit (Day 70). Another patient had elevated systolic and diastolic blood pressure (sitting and standing) at screening and baseline and at each subsequent visit. In addition, the patient had high pulse (sitting and standing) at screening, days 14 and 21 (safety visits), assessed as not clinically significant. Overall, 13 out of 18 patients in the NLX-group and 6 out of 9 patients in the placebo group had occurrences of orthostatic hypotension on one or more occasions during the study. There was no indication of increased suicidal ideation/behavior in any patient during the study.

Seven patients experienced AEs that led to dose reductions or dose interruptions, 4 on NLX-112 and 3 on placebo. One patient on NLX-112 with dry mouth, headache, palpitations and another on NLX-112 with apathy, fatigue, parkinsonism, headache, nausea, tinnitus, vertigo, arthralgia fall, discontinued the study prematurely, while the other patients with dose reductions or interruptions completed the study

A similar number of AEs were reported by subjects receiving placebo and NLX-112, and overall safety and tolerability was similar to placebo, even for patients co-treated with amantadine. For example, the proportion of patients in the NLX-112 group experiencing an adverse event was 11% higher than the placebo group (89% vs 78%, Table 6). The number of adverse events per patient in the NLX-112 group was about 11% higher than in the placebo group (71/18 vs 32/9, Table 6). The proportion of patients in the NLX-112 group experiencing dizziness was the same as the placebo group (11%, Table 7). The number of dizziness adverse events per patient in the NLX-112 group was the same as the placebo group (2/18 vs 1/9, Table 7). Adverse events were either mild or moderate, and they mostly occurred during the 4-week dose up-titration phase. Safety, as assessed by changes in vital signs, ECG or laboratory measures was good and did not differ between NLX-112 and placebo groups.

TABLE 6
Overview of adverse events (Full analysis set)

| | NLX-112 | | | Placebo | | | Total | | |
|---|---|---|---|---|---|---|---|---|---|
| | (N = 18) | | | (N = 9) | | | (N = 27) | | |
| | n (%) | | m | n (%) | | m | n (%) | | m |
| Any AE | 16 | (89%) | 71 | 7 | (78%) | 32 | 23 | (85%) | 103 |

| Any SAE | 0 | 0 | 1 | (11%) | 1 | 1 | (3.7%) | 1 |
|---|---|---|---|---|---|---|---|---|
| Congenital Anomaly or Birth Defect | 0 | 0 | 0 | | 0 | 0 | | 0 |
| Persistent or Significant Disability/Incapacity | 0 | 0 | 0 | | 0 | 0 | | 0 |
| Requires or Prolongs Hospitalization | 0 | 0 | 1 | (11%) | 1 | 1 | (3.7%) | 1 |

| Is Life Threatening | 0 | | 0 | 0 | 0 | 0 | | 0 |
|---|---|---|---|---|---|---|---|---|
| Other Medically Important Serious Event | 0 | | 0 | 0 | 0 | 0 | | 0 |
| Any AE leading to withdrawal from study | 1 | (5.6%) | 3 | 0 | 0 | 1 | (3.7%) | 3 |

| | | | | | | |
|---|---|---|---|---|---|---|
| Any AE leading to death | 0 | 0 | 0 | 0 | 0 | 0 |

Causality

| | | | | | | | | | |
|---|---|---|---|---|---|---|---|---|---|
| Unlikely Related | 11 | (61%) | 24 | 4 | (44%) | 13 | 15 | (56%) | 37 |
| Possibly Related | 15 | (83%) | 43 | 5 | (56%) | 10 | 20 | (74%) | 53 |
| Probably Related | 3 | (17%) | 4 | 3 | (33%) | 9 | 6 | (22%) | 13 |

Action taken with study drug

| Dose Not Changed | 12 | (67%) | 46 | 6 | (67%) | 21 | 18 | (67%) | 67 |
|---|---|---|---|---|---|---|---|---|---|
| Dose Reduced | 4 | (22%) | 14 | 3 | (33%) | 4 | 7 | (26%) | 18 |
| Drug Interrupted | 0 | | 0 | 1 | (11%) | 4 | 1 | (3.7%) | 4 |
| Not Applicable | 5 | (28%) | 11 | 2 | (22%) | 3 | 7 | (26%) | 14 |

Severity

| | | | | | | | | | |
|---|---|---|---|---|---|---|---|---|---|
| Mild | 14 | (78%) | 45 | 7 | (78%) | 22 | 21 | (78%) | 67 |
| Moderate | 10 | (56%) | 26 | 4 | (44%) | 7 | 14 | (52%) | 33 |

| | | | | | | | | |
|---|---|---|---|---|---|---|---|---|
| Severe | 0 | 0 | 1 | (11%) | 3 | 1 | (3.7%) | 3 |

| | | | | | | |
|---|---|---|---|---|---|---|
| Life-Threatening | 0 | 0 | 0 | 0 | 0 | 0 |
| Death | 0 | 0 | 0 | 0 | 0 | 0 |

TABLE 7
Adverse events by system organ class and preferred term (Full analysis set)

| | NLX-112 | | | Placebo | | | Total | | |
|---|---|---|---|---|---|---|---|---|---|
| System organ class | (N = 18) | | | (N = 9) | | | (N = 27) | | |
| Preferred term | n (%) | | m | n (%) | | m | n (%) | | m |
| Total | 16 | (89%) | 71 | 7 | (78%) | 32 | 23 | (85%) | 103 |
| Nervous system disorders | 10 | (56%) | 16 | 5 | (56%) | 14 | 15 | (56%) | 30 |
| Dizziness | 2 | (11%) | 2 | 1 | (11%) | 1 | 3 | (11%) | 3 |
| Dyskinesia | 2 | (11%) | 2 | 0 | | 0 | 2 | (7.4%) | 2 |
| Headache | 3 | (17%) | 4 | 2 | (22%) | 3 | 5 | (19%) | 7 |
| On and off phenomenon | 1 | (5.6%) | 1 | 0 | | 0 | 1 | (3.7%) | 1 |
| Parkinsonism | 3 | (17%) | 3 | 3 | (33%) | 7 | 6 | (22%) | 10 |
| Parosmia | 1 | (5.6%) | 1 | 0 | | 0 | 1 | (3.7%) | 1 |
| Restless legs syndrome | 2 | (11%) | 2 | 0 | | 0 | 2 | (7.4%) | 2 |
| Somnolence | 1 | (5.6%) | 1 | 1 | (11%) | 1 | 2 | (7.4%) | 2 |
| Syncope | 0 | | 0 | 1 | (11%) | 1 | 1 | (3.7%) | 1 |
| Taste disorder | 0 | | 0 | 1 | (11%) | 1 | 1 | (3.7%) | 1 |
| Psychiatric disorders | 6 | (33%) | 9 | 3 | (33%) | 3 | 9 | (33%) | 12 |
| Apathy | 1 | (5.6%) | 1 | 0 | | 0 | 1 | (3.7%) | 1 |
| Depression | 0 | | 0 | 1 | (11%) | 1 | 1 | (3.7%) | 1 |
| Dissociation | 2 | (11%) | 2 | 0 | | 0 | 2 | (7.4%) | 2 |
| Hallucination | 1 | (5.6%) | 1 | 0 | | 0 | 1 | (3.7%) | 1 |
| Illusion | 0 | | 0 | 1 | (11%) | 1 | 1 | (3.7%) | 1 |
| Insomnia | 2 | (11%) | 2 | 0 | | 0 | 2 | (7.4%) | 2 |
| Irritability | 0 | | 0 | 1 | (11%) | 1 | 1 | (3.7%) | 1 |
| Rapid eye movement sleep | 1 | (5.6%) | 1 | 0 | | 0 | 1 | (3.7%) | 1 |
| behaviour disorder | | | | | | | | | |
| Sleep disorder | 1 | (5.6%) | 1 | 0 | | 0 | 1 | (3.7%) | 1 |
| Stress | 1 | (5.6%) | 1 | 0 | | 0 | 1 | (3.7%) | 1 |
| Gastrointestinal disorders | 7 | (39%) | 10 | 1 | (11%) | 1 | 8 | (30%) | 11 |
| Diarrhoea | 1 | (5.6%) | 1 | 0 | | 0 | 1 | (3.7%) | 1 |
| Dry mouth | 1 | (5.6%) | 1 | 0 | | 0 | 1 | (3.7%) | 1 |
| Gastritis | 1 | (5.6%) | 1 | 0 | | 0 | 1 | (3.7%) | 1 |
| Nausea | 4 | (22%) | 4 | 0 | | 0 | 4 | (15%) | 4 |
| Oral pain | 1 | (5.6%) | 1 | 0 | | 0 | 1 | (3.7%) | 1 |
| Vomiting | 2 | (11%) | 2 | 1 | (11%) | 1 | 3 | (11%) | 3 |
| Infections and infestations | 3 | (17%) | 5 | 4 | (44%) | 4 | 7 | (26%) | 9 |
| COVID-19 | 0 | | 0 | 1 | (11%) | 1 | 1 | (3.7%) | 1 |
| Influenza | 1 | (5.6%) | 1 | 0 | | 0 | 1 | (3.7%) | 1 |
| Nasopharyngitis | 1 | (5.6%) | 1 | 2 | (22%) | 2 | 3 | (11%) | 3 |
| Respiratory tract infection | 1 | (5.6%) | 1 | 0 | | 0 | 1 | (3.7%) | 1 |
| Rhinitis | 0 | | 0 | 1 | (11%) | 1 | 1 | (3.7%) | 1 |
| Urinary tract infection | 1 | (5.6%) | 2 | 0 | | 0 | 1 | (3.7%) | 2 |
| Musculoskeletal and connective | 5 | (28%) | 6 | 1 | (11%) | 1 | 6 | (22%) | 7 |
| tissue disorders | | | | | | | | | |
| Arthralgia | 1 | (5.6%) | 1 | 0 | | 0 | 1 | (3.7%) | 1 |
| Back pain | 2 | (11%) | 2 | 0 | | 0 | 2 | (7.4%) | 2 |
| Muscle spasms | 1 | (5.6%) | 1 | 0 | | 0 | 1 | (3.7%) | 1 |
| Musculoskeletal stiffness | 1 | (5.6%) | 1 | 0 | | 0 | 1 | (3.7%) | 1 |
| Neck pain | 0 | | 0 | 1 | (11%) | 1 | 1 | (3.7%) | 1 |
| Pain in extremity | 1 | (5.6%) | 1 | 0 | | 0 | 1 | (3.7%) | 1 |
| General disorders and | 3 | (17%) | 3 | 2 | (22%) | 3 | 5 | (19%) | 6 |
| administration site conditions | | | | | | | | | |
| Condition aggravated | 1 | (5.6%) | 1 | 0 | | 0 | 1 | (3.7%) | 1 |
| Fatigue | 2 | (11%) | 2 | 2 | (22%) | 3 | 4 | (15%) | 5 |
| Vascular disorders | 3 | (17%) | 3 | 2 | (22%) | 2 | 5 | (19%) | 5 |
| Hypertension | 0 | | 0 | 1 | (11%) | 1 | 1 | (3.7%) | 1 |
| Orthostatic hypotension | 2 | (11%) | 2 | 1 | (11%) | 1 | 3 | (11%) | 3 |
| Systolic hypertension | 1 | (5.6%) | 1 | 0 | | 0 | 1 | (3.7%) | 1 |
| Eye disorders | 2 | (11%) | 3 | 2 | (22%) | 2 | 4 | (15%) | 5 |
| Binocular eye movement disorder | 1 | (5.6%) | 1 | 0 | | 0 | 1 | (3.7%) | 1 |
| Dry eye | 0 | | 0 | 1 | (11%) | 1 | 1 | (3.7%) | 1 |
| Erythema of eyelid | 1 | (5.6%) | 1 | 0 | | 0 | 1 | (3.7%) | 1 |
| Eye pain | 0 | | 0 | 1 | (11%) | 1 | 1 | (3.7%) | 1 |
| Swelling of eyelid | 1 | (5.6%) | 1 | 0 | | 0 | 1 | (3.7%) | 1 |
| Ear and labyrinth disorders | 2 | (11%) | 3 | 1 | (11%) | 1 | 3 | (11%) | 4 |
| Tinnitus | 1 | (5.6%) | 1 | 0 | | 0 | 1 | (3.7%) | 1 |
| Vertigo | 2 | (11%) | 2 | 1 | (11%) | 1 | 3 | (11%) | 3 |

| | | | | | | | | |
|---|---|---|---|---|---|---|---|---|
| Injury, poisoning and procedural | 3 | (17%) | 6 | 0 | 0 | 3 | (11%) | 6 |
| complications | | | | | | | | |
| Fall | 2 | (11%) | 3 | 0 | 0 | 2 | (7.4%) | 3 |
| Incorrect dose administered | 1 | (5.6%) | 1 | 0 | 0 | 1 | (3.7%) | 1 |
| Lip injury | 1 | (5.6%) | 1 | 0 | 0 | 1 | (3.7%) | 1 |
| Product communication issue | 1 | (5.6%) | 1 | 0 | 0 | 1 | (3.7%) | 1 |
| Renal and urinary disorders | 2 | (11%) | 2 | 0 | 0 | 2 | (7.4%) | 2 |
| Micturition urgency | 1 | (5.6%) | 1 | 0 | 0 | 1 | (3.7%) | 1 |
| Pollakiuria | 1 | (5.6%) | 1 | 0 | 0 | 1 | (3.7%) | 1 |
| Cardiac disorders | 1 | (5.6%) | 1 | 0 | 0 | 1 | (3.7%) | 1 |
| Palpitations | 1 | (5.6%) | 1 | 0 | 0 | 1 | (3.7%) | 1 |
| Investigations | 1 | (5.6%) | 1 | 0 | 0 | 1 | (3.7%) | 1 |
| Inflammatory marker increased | 1 | (5.6%) | 1 | 0 | 0 | 1 | (3.7%) | 1 |
| Metabolism and nutrition | 1 | (5.6%) | 1 | 0 | 0 | 1 | (3.7%) | 1 |
| disorders | | | | | | | | |
| Hyperglycaemia | 1 | (5.6%) | 1 | 0 | 0 | 1 | (3.7%) | 1 |

| | | | | | | | | |
|---|---|---|---|---|---|---|---|---|
| Skin and subcutaneous tissue | 0 | 0 | 1 | (11%) | 1 | 1 | (3.7%) | 1 |
| disorders | | | | | | | | |
| Alopecia | 0 | 0 | 1 | (11%) | 1 | 1 | (3.7%) | 1 |

| | | | | | | | | |
|---|---|---|---|---|---|---|---|---|
| Surgical and medical procedures | 1 | (5.6%) | 2 | 0 | 0 | 1 | (3.7%) | 2 |
| Cataract operation | 1 | (5.6%) | 2 | 0 | 0 | 1 | (3.7%) | 2 |

TABLE 8
Overview of adverse events by treatment period (Full analysis set)

| | NU | | | NS | | | ND | | | PU | | PS | | PD | | FU | | | T | | |
|---|---|---|---|---|---|---|---|---|---|---|---|---|---|---|---|---|---|---|---|---|---|
| | (N = 18) | | | (N = 15) | | | (N = 15) | | | (N = 9) | | (N = 8) | | (N = 8) | | (N = 27) | | | (N = 27) | | |
| | n (%) | | m | n (%) | | m | n (%) | | m | n (%) | m | n (%) | m | n (%) | m | n (%) | | m | n (%) | | m |
| Any AE | 14 | (78) | 52 | 8 | (53) | 11 | 4 | (27) | 6 | 7 (78) | 17 | 4 (50) | 7 | 4 (50) | 7 | 3 | (11) | 3 | 23 | (85) | 103 |

| Any SAE | 0 | | 0 | 0 | 0 | 0 | 0 | 0 | 0 | 1 (13) | 1 | 0 | 0 | 0 | 0 | 1 | (3.7) | 1 |
|---|---|---|---|---|---|---|---|---|---|---|---|---|---|---|---|---|---|---|
| CA or BD | 0 | | 0 | 0 | 0 | 0 | 0 | 0 | 0 | 0 | 0 | 0 | 0 | 0 | 0 | 0 | | 0 |
| PSD/I | 0 | | 0 | 0 | 0 | 0 | 0 | 0 | 0 | 0 | 0 | 0 | 0 | 0 | 0 | 0 | | 0 |
| RPH | 0 | | 0 | 0 | 0 | 0 | 0 | 0 | 0 | 1 (13) | 1 | | 0 | 0 | 0 | 1 | (3.7) | 1 |
| ILT | 0 | | 0 | 0 | 0 | 0 | 0 | 0 | 0 | 0 | 0 | 0 | 0 | 0 | 0 | 0 | | 0 |
| OMISE | 0 | | 0 | 0 | 0 | 0 | 0 | 0 | 0 | 0 | 0 | 0 | 0 | 0 | 0 | 0 | | 0 |
| Any AE leading | 1 | (5.6) | 3 | 0 | 0 | 0 | 0 | 0 | 0 | 0 | 0 | 0 | 0 | 0 | 0 | 1 | (3.7) | 3 |
| to WFS | | | | | | | | | | | | | | | | | | |

| | | | | | | | | | | | | | | | | |
|---|---|---|---|---|---|---|---|---|---|---|---|---|---|---|---|---|
| Any AE leading | 0 | 0 | 0 | 0 | 0 | 0 | 0 | 0 | 0 | 0 | 0 | 0 | 0 | 0 | 0 | 0 |
| to death | | | | | | | | | | | | | | | | |

Causality

| | | | | | | | | | | | | | | | | | | | | | |
|---|---|---|---|---|---|---|---|---|---|---|---|---|---|---|---|---|---|---|---|---|---|
| Unlikely Related | 10 | (56) | 17 | 3 | (20) | 3 | 2 | (13) | 2 | 3 (33) | 4 | 2 (25) | 2 | 3 (38) | 6 | 3 | (11) | 3 | 15 | (56) | 37 |

| | | | | | | | | | | | | | | | | | | | | |
|---|---|---|---|---|---|---|---|---|---|---|---|---|---|---|---|---|---|---|---|---|
| Possibly Related | 11 | (61) | 33 | 5 | (33) | 7 | 2 | (13) | 3 | 3 (33) | 5 | 2 (25) | 4 | 1 (13) | 1 | 0 | 0 | 20 | (74) | 53 |
| Probably Related | 2 | (11) | 2 | 1 | (6.7) | 1 | 1 | (6.7) | 1 | 3 (33) | 8 | 1 (13) | 1 | 0 | 0 | 0 | 0 | 6 | (22) | 13 |

Action taken with study drug

| | | | | | | | | | | | | | | | | | | | | | |
|---|---|---|---|---|---|---|---|---|---|---|---|---|---|---|---|---|---|---|---|---|---|
| Dose Not Changed | 10 | (56) | 30 | 7 | (47) | 9 | 4 | (27) | 6 | 6 (67) | 11 | 3 (38) | 5 | 3 (38) | 5 | 1 | (3.7) | 1 | 18 | (67) | 67 |

| Dose Reduced | 3 | (17) | 13 | 1 | (6.7) | 1 | 0 | 0 | 1 (11) | 2 | 2 (25) | 2 | 0 | 0 | 0 | | 0 | 7 | (26) | 18 |
|---|---|---|---|---|---|---|---|---|---|---|---|---|---|---|---|---|---|---|---|---|
| Drug Interrupted | 0 | | 0 | 0 | | 0 | 0 | 0 | 1 (11) | 4 | 0 | 0 | 0 | 0 | 0 | | 0 | 1 | (3.7) | 4 |
| Not Applicable | 5 | (28) | 9 | 1 | (6.7) | 1 | 0 | 0 | 0 | 0 | 0 | 0 | 2 (25) | 2 | 2 | (7.4) | 2 | 7 | (26) | 14 |

Severity

| | | | | | | | | | | | | | | | | | | | | | |
|---|---|---|---|---|---|---|---|---|---|---|---|---|---|---|---|---|---|---|---|---|---|
| Mild | 11 | (61) | 35 | 4 | (27) | 5 | 1 | (6.7) | 3 | 7 (78) | 12 | 3 (38) | 4 | 3 (38) | 6 | 2 | (7.4) | 2 | 21 | (78) | 67 |
| Moderate | 7 | (39) | 17 | 4 | (27) | 6 | 3 | (20) | 3 | 2 (22) | 3 | 2 (25) | 2 | 1 (13) | 1 | 1 | (3.7) | 1 | 14 | (52) | 33 |

| | | | | | | | | | | | | | | | | | |
|---|---|---|---|---|---|---|---|---|---|---|---|---|---|---|---|---|---|
| Severe | 0 | 0 | 0 | 0 | 0 | 0 | 1 (11) | 2 | 1 (13) | 1 | 0 | 0 | 0 | 0 | 1 | (3.7) | 3 |

| | | | | | | | | | | | | | | | | |
|---|---|---|---|---|---|---|---|---|---|---|---|---|---|---|---|---|
| Life-Threatening | 0 | 0 | 0 | 0 | 0 | 0 | 0 | 0 | 0 | 0 | 0 | 0 | 0 | 0 | 0 | 0 |
| Death | 0 | 0 | 0 | 0 | 0 | 0 | 0 | 0 | 0 | 0 | 0 | 0 | 0 | 0 | 0 | 0 |

TABLE 9
Adverse Events by treatment period (Full analysis set)

| | NLX-112 | | | NLX-112 | | | NLX-112 | | | Placebo | | Placebo | | Placebo | | | | | | |
|---|---|---|---|---|---|---|---|---|---|---|---|---|---|---|---|---|---|---|---|---|
| System | up- | | | stable | | | down- | | | up- | | stable | | down- | | Follow- | | | | |
| organ class | titration | | | dosing | | | titration | | | titration | | dosing | | titration | | up | | Total | | |
| Preferred | (N = 18) | | | (N = 15) | | | (N = 15) | | | (N = 8) | | (N = 8) | | (N = 8) | | (N = 27) | | (N = 27) | | |
| term | n (%) | | m | n (%) | | m | n (%) | | m | n (%) | m | n (%) | m | n (%) | m | n (%) | m | n (%) | | m |
| Total | 14 | (78) | 52 | 8 | (53) | 11 | 4 | (27) | 6 | 7 (78) | 17 | 4 (50) | 7 | 4 (50) | 7 | 3 (11) | 3 | 23 | (85) | 103 |
| Dizziness | 2 | (11) | 2 | 0 | | 0 | 0 | | 0 | 1 (11) | 1 | 0 | 0 | 0 | 0 | 0 | 0 | 3 | (11) | 3 |
| Dyskinesia | 2 | (11) | 2 | 0 | | 0 | 0 | | 0 | 0 | 0 | 0 | 0 | 0 | 0 | 0 | 0 | 2 | (7.4) | 2 |
| Headache | 2 | (11) | 2 | 1 | (6.7) | 1 | 1 | (6.7) | 1 | 2 (22) | 2 | 0 | 0 | 1 (13) | 1 | 0 | 0 | 5 | (19) | 7 |
| Parkinsonism | 3 | (17) | 3 | 0 | | 0 | 0 | | 0 | 3 (33) | 5 | 1 (13) | 1 | 1 (13) | 1 | 0 | 0 | 6 | (22) | 10 |
| Restless legs | 1 | (5.6) | 1 | 1 | (6.7) | 1 | 0 | | 0 | 0 | 0 | 0 | 0 | 0 | 0 | 0 | 0 | 2 | (7.4) | 2 |
| syndrome | | | | | | | | | | | | | | | | | | | | |
| Dissociation | 1 | (5.6) | 1 | 1 | (6.7) | 1 | 0 | | 0 | 0 | 0 | 0 | 0 | 0 | 0 | 0 | 0 | 2 | (7.4) | 2 |
| Insomnia | 1 | (5.6) | 1 | 0 | | 0 | 1 | (6.7) | 1 | 0 | 0 | 0 | 0 | 0 | 0 | 0 | 0 | 2 | (7.4) | 2 |
| Nausea | 2 | (11) | 2 | 1 | (6.7) | 1 | 1 | (6.7) | 1 | 0 | 0 | 0 | 0 | 0 | 0 | 0 | 0 | 4 | (15) | 4 |

| Vomiting | 2 | (11) | 2 | 0 | 0 | 0 | | 0 | 1 (11) | 1 | 0 | 0 | 0 | 0 | 0 | 0 | 3 | (11) | 3 |
|---|---|---|---|---|---|---|---|---|---|---|---|---|---|---|---|---|---|---|---|
| Back pain | 1 | (5.6) | 1 | 0 | 0 | 1 | (6.7) | 1 | 0 | 0 | 0 | 0 | 0 | 0 | 0 | 0 | 2 | (7.4) | 2 |

| Fatigue | 1 | (5.6) | 1 | 1 | (6.7) | 1 | 0 | 0 | 1 (11) | 1 | 2 (25) | 2 | 0 | 0 | 0 | 0 | 4 | (15) | 5 |
|---|---|---|---|---|---|---|---|---|---|---|---|---|---|---|---|---|---|---|---|
| Orthostatic | 0 | | 0 | 2 | (13) | 2 | 0 | 0 | 0 | 0 | 1 (13) | 1 | 0 | 0 | 0 | 0 | 3 | (11) | 3 |
| hypotension | | | | | | | | | | | | | | | | | | | |
| Vertigo | 2 | (11) | 2 | 0 | | 0 | 0 | 0 | 1 (11) | 1 | 0 | 0 | 0 | 0 | 0 | 0 | 3 | (11) | 3 |
| Fall | 1 | (5.6) | 2 | 1 | (6.7) | 1 | 0 | 0 | 0 | 0 | 0 | 0 | 0 | 0 | 0 | 0 | 2 | (7.4) | 3 |

### Efficacy

Efficacy assessments were made at baseline, at the end of the up-titration period (day 28) and following stable dose treatment (day 42). There were no significant differences between the NLX-112 group and the placebo group in terms of change from baseline to Day 28 or Day 42 in either UDysRS total score or UDysRS total objective (Parts 3 and 4) score.

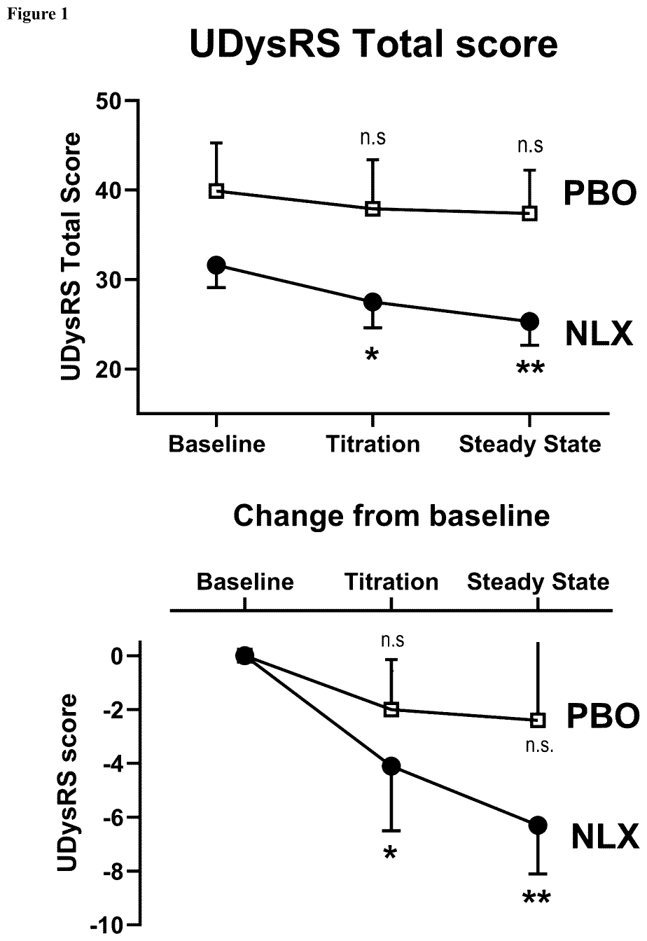

The mean UDysRS total score (Parts 1-4), based on the assessment with most severe dyskinesia after L-DOPA challenge, at baseline was 31.6 in the NLX-112 group and 39.9 in the placebo group out of a possible maximum of 104. A significant reduction in UDysRS total score compared to baseline was seen within the NLX-112 group on days 28 and 42; there were no significant changes from baseline for the placebo group (Table 10 and in FIG. 1). For example, in motor assessment at Day 28 NLX-112 reduced UDysRS total score (Parts 1-4) by −4.1 points (p=0.0281), whereas changes in the placebo group were not significant (−2.0). At Day 42, a greater reduction UDysRS scores by NLX-112 was observed: total score was reduced by −6.3 points (p=0.0016), whereas changes in the placebo group were not significant (−2.4) (See Table 10, below, and FIG. 1). Similarly, a significant reduction in UDysRS total score based on the average of 3 assessments (30, 60 and 90 minutes) was seen within the NLX-112 group on both days 28 and 42; there were no significant changes from baseline for the placebo group (Table 13).

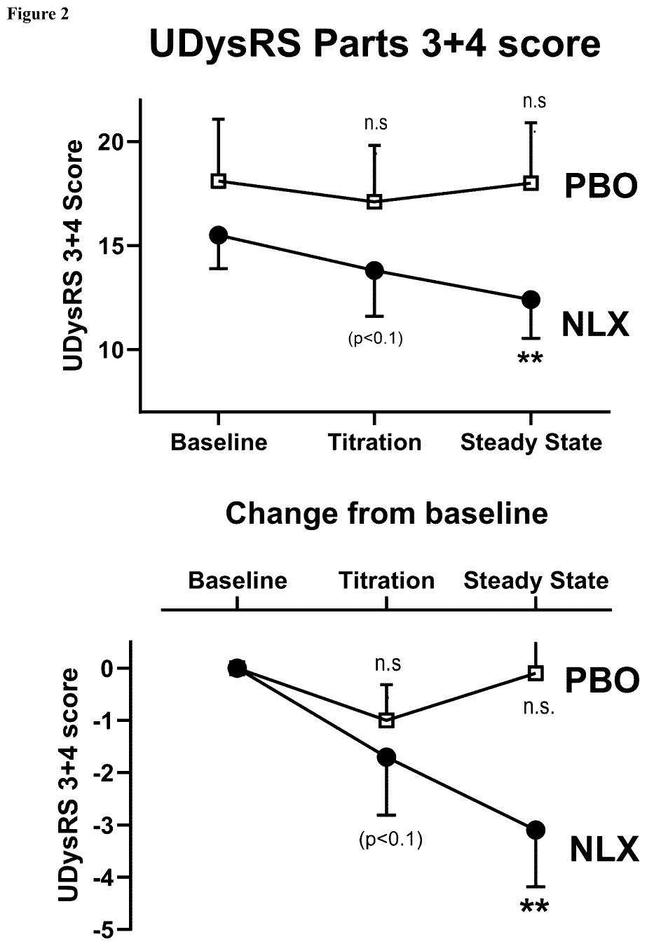

The mean UDysRS total objective score (Parts 3+4), based on the assessment with most severe dyskinesia, was 15.5 at baseline in the NLX-112 group and 18.1 in the placebo group out of a possible maximum score of 44. A significant reduction compared to baseline in UDysRS total objective score was seen within the NLX-112 group on Day 42 but not on Day 28. There were no significant changes within the placebo group. For example, in motor assessment at Day 28 NLX-112 reduced UDysRS part 3+4 score (dyskinesia disability) by −1.7 points (not statistically significant) compared to baseline, whereas changes in the placebo group were not significant (−1.0). At Day 42, a greater reduction UDysRS scores by NLX-112 was observed: Part 3+4 score was reduced by −3.1 points (p=0.0038) compared to baseline, whereas changes in the placebo group were not significant (−0.1) (See Table 11, below, and FIG. 2).

Similarly, a significant reduction in UDysRS total objective score based on the average of 3 assessments (30, 60 and 90 minutes) was seen within the NLX-112 group both on Days 28 and 42. There were no significant changes from baseline within the placebo group. For example, in motor assessment at Day 28 NLX-112 reduced UDysRS Part 3+4 score (dyskinesia disability) by −2.49 points (p=0.0065) compared to baseline, whereas changes in the placebo group were not significant (−1.05). At Day 42, a greater reduction UDysRS scores by NLX-112 was observed: Part 3+4 score was reduced by −3.31 points (p=0.0005) compared to baseline, whereas changes in the placebo group were not significant (−0.95) (See Table 12, below).

Five of the patients in the NLX-112 group were also receiving amantadine as part of their stable treatment. Three of these patients showed decreases in UDysRS total score and UDysRS total objective score (Parts 3+4) (see Table 23).

Overall, NLX-112 was safe, well tolerated and significantly reduced LID in an apparently treatment-duration dependent manner. These results were surprising for several reasons: a) the baseline UDysRS score in the NLX-112 group was lower than the placebo group, which limited the potential effect size; b) the patients received a 150% L-DOPA challenge, which increased the potential to overwhelm the effect of NLX-112; c) the group size was small (n=15), which increased the magnitude of effect required for statistical significance; d) the steady state duration was only two weeks, which limited the drug exposure and time to observe an effect, and e) the maximum dose was limited to 2 mg.

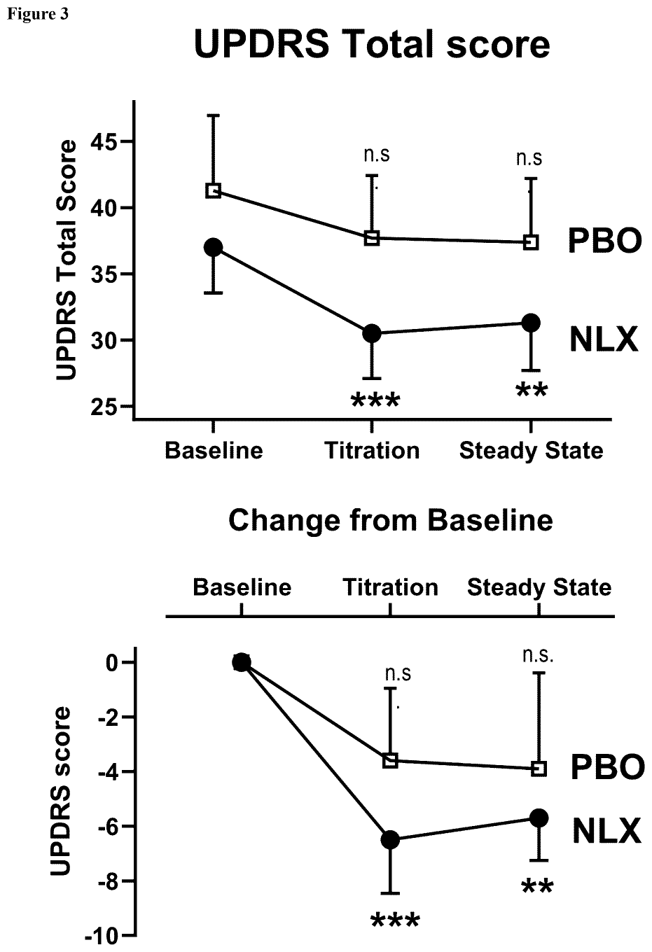

Surprisingly, not only did NLX-112 not interfere with the beneficial anti-parkinsonian effects of L-DOPA, NLX-112 did not worsen, and actually enhanced, the beneficial effects of L-DOPA. The mean UPDRS total score (all Parts) at baseline was 37.0 in the NLX-group and 41.3 in the placebo group out of a possible maximum of 199. A significant reduction compared to baseline was seen within the NLX-112 group on days 28 and 42 (Tables 14 and 22, FIG. 3), whereas in the placebo group, there were no significant changes from baseline. There were no significant differences between the NLX-112 group and the placebo group in terms of absolute change from baseline to days 28 or 42. For example, Table 14 (below) and FIG. 3 show that at Day 28 NLX-112 reduced UPDRS total score (Parts 1-4) by −6.5 points (p=0.0005) compared to baseline, whereas changes in the placebo group were not significant (−3.6). At Day 42, NLX-112 reduced UPDRS total score by −5.7 points (p=0.0021) compared to baseline, whereas changes in the placebo group were not significant (−3.9).

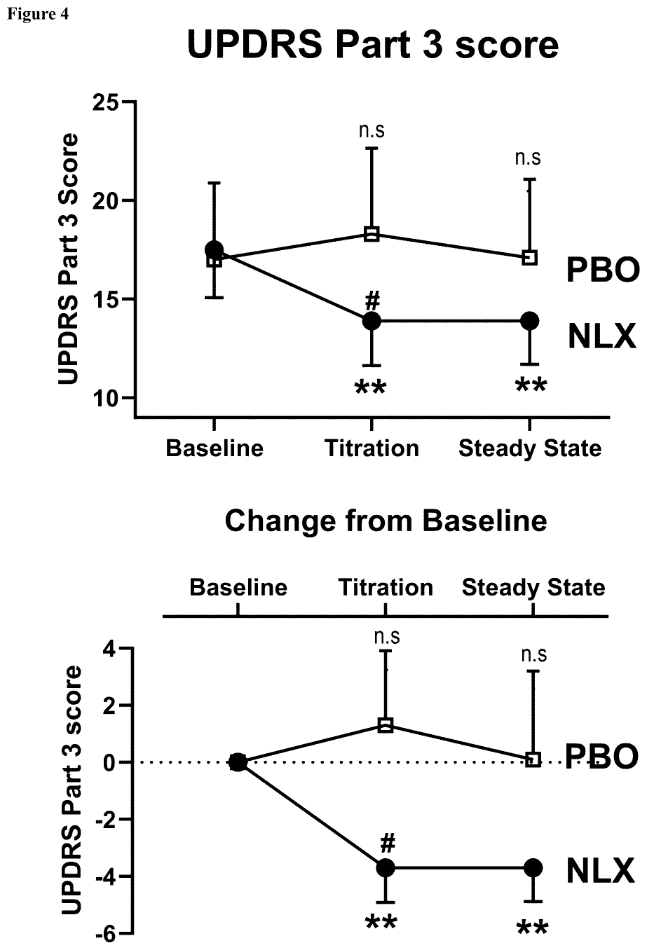

For Part 3 of the UPDRS, which assesses parkinsonism, the mean total score at baseline was 17.5 in the NLX-group and 17.0 in the placebo group out of a possible maximum of 108. A statistically significant reduction compared to baseline was seen in the NLX-112 group on days 28 and 42 while there were no significant changes in the placebo group. There was also a significant difference between the NLX-112 and placebo groups in terms of absolute change from baseline to Day 28 but not to Day 42 (see Table 22). For example, Tables 15 and 22 (below) and FIG. 4 show that at Day 28 NLX-112 reduced UPDRS Part 3 score (motor disability) by −3.7 points (p=0.0072) compared to baseline, whereas changes in the placebo group were not significant (+1.3). At Day 42, NLX-112 reduced UPDRS Part 3 score by −3.7 points (p=0.0072) compared to baseline, whereas changes in the placebo group were not significant (+0.1).

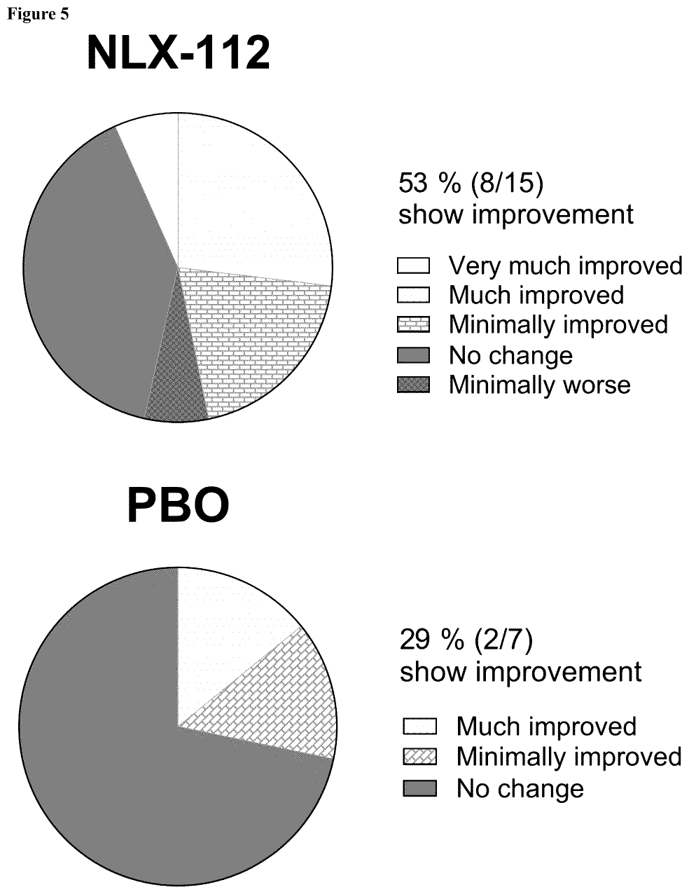

These results were surprising because the patients received a 150% L-DOPA challenge, which could have been expected to adequately treat their parkinsonian symptoms, leaving less room for any observable improvement by NLX-112. The improvement observed in UPDRS Part 3 scores is particularly surprising because UPDRS Part 3 measures the motor symptoms of Parkinson's disease, and other 5-HT_{1A} agonists (e.g., sarizotan, tandospirone) exhibited no beneficial effect or even the opposite effect, negatively impacting motor symptoms of Parkinson's disease. This is the first time a drug targeting the serotonin system has shown both anti-LID and anti-parkinsonian activity and suggests that NLX-112 could constitute a first-in-class dual-acting therapeutic for movement symptoms of PD. NLX-112 also improved the Clinical General Impression of Change (CGI-C) score at day 42 (visit 7) compared to baseline (day 1, visit 2). As shown in FIG. 5, 53% of patients showed improvement compared to 29% in the placebo group. Together, these data suggest that NLX-112 could be used to reduce the L-DOPA dose necessary for effective treatment of Parkinson's symptoms and/or as an alternative to dopamine agonists such as rotigotine, pramipexole, and ropinirole.

Tables 10-23 provide efficacy data from the study. In each Table, n=Number of observations; SD=Standard deviation; SAS program=descstat.sas; ^{a)}=Linear mixed model (LMM) of change from baseline with treatment, visit and the interaction treatment*visit as fixed categorical effects and subject within treatment as a random effect. The baseline value of the dependent variable has been included in the model as a continuous covariate. Kenward-Roger's improved approximation for degrees of freedom (2009) has been used; ^{b)}=p-value for the difference between NLX-112 at the assessment timepoint and at baseline; ^{c)}=p-value for the difference between placebo at the assessment timepoint and at baseline; ^{d)}=p-value for the difference between NLX-112 and placebo absolute change from baseline at the assessment timepoint. Table 16 provides the UDysRS total score for the Full Analysis Set (FAS), with Part 3 based on the highest score of the 3 assessment sessions (30, 60, 90 min after L-DOPA dose). Table 19 provides the UDysRS total score for the Full Analysis Set (FAS), with Part 3 based on the average score of the 3 assessment sessions (30, 60, 90 min after L-DOPA dose).

Also surprising is the observation that anti-dyskinetic efficacy was higher at day 42 than day 28. Indeed, a loss of efficacy could have been predicted because 5-HT1A receptors are known to be desensitized upon extended periods of activation (Assie et al., “Rapid desensitization of somatodendritic 5-HT1A receptors by chronic administration of the high-efficacy 5-HT1A agonist, F13714: a microdialysis study in the rat,” Br J Pharmacol 2006, 149, 170-8; Blier et al., “Role of somatodendritic 5-HT autoreceptors in modulating 5-HT neurotransmission,” Annals of the New York Academy of Sciences 1998, 861, 204-16). Although a rat study with NLX-112 showed that it did maintain its antidyskinetic efficacy against L-DOPA-induced dyskinesia over a 2-week treatment period, no increase in efficacy was observed over that time (McCreary et al., “The novel 5-HT1A receptor agonist, NLX-112 reduces 1-DOPA-induced abnormal involuntary movements in rat: A chronic administration study with microdialysis measurements,” Neuropharmacology, 2016, 105, 651-660). In contrast, Tables 9-13 show that NLX-112 more efficaciously reduced L-DOPA-induced dyskinesia at day 42 than at day 28.

Finally, the distinct timeline of the antidyskinetic effects (which accentuate over time, see Tables 9-13) and its anti-parkinsonian effects (which were similar at days 28 and 42, see Tables 14 and 15) of NLX-112 are surprising. This difference suggests that there may be different mechanisms involved.

The mean UDysRS score for the sum of Part 3 items (objective impairment assessment), based on the assessment with most severe dyskinesia, was 24.7 at baseline in the NLX-112 group and 28.9 in the placebo group out of a possible maximum score of 112. A significant reduction in the sum of Part 3 items compared to baseline was seen within the NLX 112 group on both days 28 and 42. There were no significant changes at either visit within the placebo group and there were no significant differences between the NLX-112 group and the placebo group. A significant reduction in the sum of Part 3 items based on the average of all 3 assessments (30, 60 and 90 minutes) was seen within the NLX 112 group both on Day 28 and on Day 42. There were no significant changes from baseline within the placebo group. There was a significantly greater change from baseline for the NLX-112 group than for the placebo group at both day 28 and at day 42. (See Table 22)

For UPDRS Part 1, which assesses non-motor experiences of daily living, there were no statistically significant changes from baseline within either treatment group. For UPDRS Part 2, which assesses motor experiences of daily living, a statistically significant reduction compared to baseline was seen within the NLX-112 group on Day 28, but not on Day 42. In the placebo group no significant changes were observed either on Day 28 or on Day 42. For UPDRS Part IV, which provides a subjective patient assessment of motor complications (including dyskinesias) during previous week, a significant reduction compared to baseline was seen in the NLX-112 group on days 28 and 42. In the placebo group, a significant reduction compared to baseline was seen on Day 28 but not on Day 42. (See Table 22)

Of the five patients in the NLX-112 group that were also receiving amantadine, three of them showed decreases in UPDRS total score and UPDRS Part 3 (see Table 23).

In Tables 10 and 11, data is based on Per protocol analysis set. The session marked as most severe for Part III and IV have been used. For Part III, the highest score for each individual questionnaire item has been used. All UDysRS item scores for Part I, II, III and IV have been summarized per patient and assessment timepoint and used as input data to the table. In Table 12, data is based on Per protocol analysis set. The patient average of all assessment sessions for Part III and IV has been used. For Part III, the highest score for each individual questionnaire item has been used. All UDysRS item scores for Part III and IV have been summarized per patient and assessment timepoint and used as input data to the table. In Table 13, data is based on Per protocol analysis set. The patient average of all assessment sessions for Part III and IV has been used. For Part III, the highest score for each individual questionnaire item has been used. All UDysRS item scores for Part I, II, III and IV have been summarized per patient and assessment timepoint and used as input data to the table. In Tables 14 and 15, data is based on Per protocol analysis set.

TABLE 10
UDysRS total score for all Parts (Per protocol analysis set)

| | | Assessment | | NLX-112 | | Placebo | | Total | |
|---|---|---|---|---|---|---|---|---|---|
| Calculated score | Result category | timepoint | | (N = 15) | | (N = 7) | | (N = 22) | |
| UDysRS Parts 1, | Collected value | Visit 2 Baseline | n | 15 | | 7 | | 22 | |
| 2, 3, and 4 most | | | Mean (SD) | 31.6 | (9.7) | 39.9 | (14.2) | 34.2 | (11.6) |
| severe | | | Median (Min, Max) | 31.0 | (13, 47) | 34.0 | (27, 66) | 33.0 | (13, 66) |
| | | Visit 6 Clinic | n | 15 | | 7 | | 22 | |
| | | Efficacy Visit | Mean (SD) | 27.5 | (11.2) | 37.9 | (14.5) | 30.8 | (13.0) |
| | | | Median (Min, Max) | 28.0 | (14, 55) | 35.0 | (21, 66) | 29.0 | (14, 66) |
| | | Visit 7 Clinic | n | 15 | | 7 | | 22 | |
| | | Efficacy Visit | Mean (SD) | 25.3 | (10.2) | 37.4 | (12.8) | 29.1 | (12.2) |
| | | | Median (Min, Max) | 24.0 | (11, 51) | 35.0 | (22, 57) | 26.5 | (11, 57) |
| | Absolute change | Visit 6 Clinic | n | 15 | | 7 | | 22 | |
| | from baseline | Efficacy Visit | Mean (SD) | −4.1 | (9.3) | −2.0 | (4.9) | −3.4 | (8.1) |
| | | | Median (Min, Max) | −2.0 | (−21,11) | −1.0 | (−7, 6) | −1.5 | (−21, 11) |
| | | | LMM^{a} p-value | 0.0281^{b} | | 0.7953^{c} | | 0.3018^{d} | |
| | | Visit 7 Clinic | n | 15 | | 7 | | 22 | |
| | | Efficacy Visit | Mean (SD) | −6.3 | (7.0) | −2.4 | (9.7) | −5.1 | (7.9) |
| | | | Median (Min, Max) | −8.0 | (−19, 7) | −4.0 | (−15, 16) | −5.5 | (−19, 16) |
| | | | LMM^{a} p-value | 0.0016^{b} | | 0.6886^{c} | | 0.1301^{d} | |
| | Relative change | Visit 6 Clinic | n | 15 | | 7 | | 22 | |
| | from baseline (%) | Efficacy Visit | Mean (SD) | −10.2 | (28.0) | −5.07 | (13.8) | −8.58 | (24.1) |
| | | | Median (Min, Max) | −6.45 | (−51, 38) | −3.70 | (−22, 18) | −5.08 | (−51, 38) |

| Visit 7 Clinic | n | 15 | | 7 | | 22 | |
|---|---|---|---|---|---|---|---|
| Efficacy Visit | Mean (SD) | −19.0 | (22.6) | −3.99 | (25.5) | −14.2 | (24.0) |
| | Median (Min, Max) | −25.8 | (−50, 31) | −13.6 | (−30, 48) | −16.7 | (−50, 48) |

TABLE 11
UDysRS total score for Part III and IV - Most severe assessment session
and highest questionnaire item score (Per protocol analysis set)

| Calculated | | Assessment | | NLX-112 | | Placebo | | Total | |
|---|---|---|---|---|---|---|---|---|---|
| score | Result category | timepoint | | (N = 15) | | (N = 7) | | (N = 22) | |
| UDysRS | Collected value | Visit 2 | n | 15 | | 7 | | 22 | |
| Parts 3 and 4 | | Baseline | Mean (SD) | 15.5 | (6.2) | 18.1 | (7.9) | 16.4 | (6.7) |
| most severe | | | Median (Min, Max) | 14.0 | (6, 29) | 17.0 | (11, 34) | 15.5 | (6, 34) |
| | | Visit 6 Clinic | n | 15 | | 7 | | 22 | |
| | | Efficacy Visit | Mean (SD) | 13.8 | (8.5) | 17.1 | (7.2) | 14.9 | (8.1) |
| | | | Median (Min, Max) | 12.0 | (2, 36) | 16.0 | (10, 31) | 13.0 | (2, 36) |
| | | Visit 7 Clinic | n | 15 | | 7 | | 22 | |
| | | Efficacy Visit | Mean (SD) | 12.4 | (7.2) | 18.0 | (7.7) | 14.2 | (7.7) |
| | | | Median (Min, Max) | 11.0 | (5, 31) | 16.0 | (10, 32) | 12.0 | (5, 32) |
| | Absolute change | Visit 6 Clinic | n | 15 | | 7 | | 22 | |
| | from baseline | Efficacy Visit | Mean (SD) | −1.7 | (4.3) | −1.0 | (1.8) | −1.5 | (3.6) |
| | | | Median (Min, Max) | −1.0 | (−10, 7) | −2.0 | (−3, 2) | −1.5 | (−10, 7) |
| | | | LMM^{a} p-value | 0.0958^{b} | | 0.5157^{c} | | 0.6789^{d} | |
| | | Visit 7 Clinic | n | 15 | | 7 | | 22 | |
| | | Efficacy Visit | Mean (SD) | −3.1 | (4.2) | −0.1 | (3.8) | −2.2 | (4.2) |
| | | | Median (Min, Max) | −4.0 | (−11, 3) | −1.0 | (−5, 7) | −2.0 | (−11, 7) |
| | | | LMM^{a} p-value | 0.0038^{b} | | 0.9336^{c} | | 0.1054^{d} | |
| | Relative change | Visit 6 Clinic | n | 15 | | 7 | | 22 | |
| | from baseline (%) | Efficacy Visit | Mean (SD) | −15.8 | (26.9) | −4.69 | (12.4) | −12.3 | (23.5) |
| | | | Median (Min, Max) | −8.33 | (−67, 24) | −8.82 | (−17, 18) | −8.58 | (−67, 24) |

| Visit 7 Clinic | n | 15 | | 7 | | 22 | |
|---|---|---|---|---|---|---|---|
| Efficacy Visit | Mean (SD) | −21.0 | (24.5) | 0.706 | (21.7) | −14.1 | (25.4) |
| | Median (Min, Max) | −31.6 | (−55, 17) | −5.26 | (−24, 41) | −10.0 | (−55, 41) |

TABLE 12
UDysRS total score for Part III and IV - Average of assessment sessions
and highest questionnaire item score (Per protocol analysis set)

| Calculated | | Assessment | | NLX-112 | | Placebo | | Total | |
|---|---|---|---|---|---|---|---|---|---|
| score | Result category | timepoint | | (N = 15) | | (N = 7) | | (N = 22) | |
| UDysRS | Collected value | Visit 2 | n | 15 | | 7 | | 22 | |
| Parts 3 and 4 | | Baseline | Mean (SD) | 14.09 | (6.55) | 17.38 | (7.50) | 15.14 | (6.87) |
| average | | | Median (Min, Max) | 13.00 | (3.7, 27.0) | 17.00 | (11.0, 32.7) | 13.17 | (3.7, 32.7) |
| | | Visit 6 Clinic | n | 15 | | 7 | | 22 | |
| | | Efficacy Visit | Mean (SD) | 11.60 | (7.48) | 16.33 | (6.31) | 13.11 | (7.33) |
| | | | Median (Min, Max) | 10.33 | (0.7, 30.0) | 14.00 | (10.3, 28.7) | 11.33 | (0.7, 30.0) |
| | | Visit 7 Clinic | n | 15 | | 7 | | 22 | |
| | | Efficacy Visit | Mean (SD) | 10.78 | (6.88) | 16.43 | (7.58) | 12.58 | (7.43) |
| | | | Median (Min, Max) | 9.67 | (2.0, 27.7) | 14.67 | (9.3, 30.7) | 11.00 | (2.0, 30.7) |
| | Absolute change | Visit 6 Clinic | n | 15 | | 7 | | 22 | |
| | from baseline | Efficacy Visit | Mean (SD) | −2.49 | (4.22) | −1.05 | (1.85) | −2.03 | (3.65) |
| | | | Median (Min, Max) | −1.67 | (−12.0, 3.0) | −0.67 | (−4.0, 0.7) | −1.00 | (−12.0, 3.0) |
| | | | LMM^{a} p-value | 0.0065^{b} | | 0.5230^{c} | | 0.2875^{d} | |
| | | Visit 7 Clinic | n | 15 | | 7 | | 22 | |
| | | Efficacy Visit | Mean (SD) | −3.31 | (3.38) | −0.95 | (2.93) | −2.56 | (3.37) |
| | | | Median (Min, Max) | −3.33 | (−9.3, 1.0) | −1.00 | (−5.0, 4.7) | −2.17 | (−9.3, 4.7) |
| | | | LMM^{a} p-value | 0.0005^{b} | | 0.5703^{c} | | 0.1070^{d} | |
| | Relative change | Visit 6 Clinic | n | 15 | | 7 | | 22 | |
| | from baseline (%) | Efficacy Visit | Mean (SD) | −17.3 | (35.6) | −4.59 | (9.27) | −13.3 | (30.1) |
| | | | Median (Min, Max) | −20.0 | (−88, 64) | −3.39 | (−18, 6.1) | −7.41 | (−88, 64) |

| Visit 7 Clinic | n | 15 | | 7 | | 22 | |
|---|---|---|---|---|---|---|---|
| Efficacy Visit | Mean (SD) | −25.3 | (23.3) | −5.68 | (16.9) | −19.1 | (23.1) |
| | Median (Min, Max) | −31.5 | (−63, 9.1) | −6.12 | (−25, 27) | −14.5 | (−63, 27) |

TABLE 13
UDysRS total score for all Parts - Average of assessment sessions Part 3 and Part 4 (Per protocol analysis set)

| Calculated | Result | Assessment | | NLX-112 | | Placebo | | Total | |
|---|---|---|---|---|---|---|---|---|---|
| score | category | timepoint | | (N = 15) | | (N = 7) | | (N = 22) | |
| UDysRS | Collected | Visit 2 | n | 15 | | 7 | | 22 | |
| Parts 1, 2, | value | Baseline | Mean (SD) | 30.16 | (10.45) | 39.10 | (13.75) | 33.00 | (12.04) |
| 3, and 4 | | | Median (Min, Max) | 29.33 | (10.7, 46.0) | 33.00 | (26.7, 64.7) | 31.17 | (10.7, 64.7) |
| average | | Visit 6 Clinic | n | 15 | | 7 | | 22 | |
| | | Efficacy Visit | Mean (SD) | 25.33 | (10.11) | 37.05 | (13.91) | 29.06 | (12.43) |
| | | | Median (Min, Max) | 25.33 | (12.7, 49.0) | 36.67 | (21.3, 63.7) | 25.83 | (12.7, 63.7) |
| | | Visit 7 Clinic | n | 15 | | 7 | | 22 | |
| | | Efficacy Visit | Mean (SD) | 23.64 | (9.96) | 35.86 | (12.43) | 27.53 | (12.01) |
| | | | Median (Min, Max) | 23.00 | (10.3, 47.7) | 33.67 | (21.0, 55.7) | 25.17 | (10.3, 55.7) |
| | Absolute | Visit 6 Clinic | n | 15 | | 7 | | 22 | |
| | change | Efficacy Visit | Mean (SD) | −4.82 | (9.32) | −2.05 | (3.56) | −3.94 | (7.95) |
| | from | | Median (Min, Max) | −3.00 | (−21.0, 7.3) | −2.33 | (−5.7, 4.0) | −2.67 | (−21.0, 7.3) |
| | baseline | | LMM^{a} p-value | 0.0053^{b} | | 0.8823^{c} | | 0.1405^{d} | |
| | | Visit 7 Clinic | n | 15 | | 7 | | 22 | |
| | | Efficacy Visit | Mean (SD) | −6.51 | (6.81) | −3.24 | (9.01) | −5.47 | (7.52) |
| | | | Median (Min, Max) | −7.67 | (−19.3, 5.7) | −4.33 | (−15.0, 13.7) | −5.83 | (−19.3, 13.7) |
| | | | LMM^{a} p-value | 0.0004^{b} | | 0.5695^{c} | | 0.1070^{d} | |
| | Relative | Visit 6 Clinic | n | 15 | | 7 | | 22 | |
| | change | Efficacy Visit | Mean (SD) | −9.33 | (35.5) | −5.56 | (10.6) | −8.13 | (29.6) |
| | from | | Median (Min, Max) | −10.6 | (−57, 69) | −8.64 | (−20, 12) | −9.62 | (−57, 69) |
| | baseline | Visit 7 Clinic | n | 15 | | 7 | | 22 | |
| | (%) | Efficacy Visit | Mean (SD) | −19.5 | (22.3) | −6.53 | (23.8) | −15.4 | (23.1) |
| | | | Median (Min, Max) | −21.9 | (−51, 25) | −13.9 | (−31, 41) | −18.5 | (−51, 41) |

TABLE 14
UPDRS total scores for all Parts (Per protocol analysis set)

| Calculated | Result | Assessment | | NLX-112 | | Placebo | | Total | |
|---|---|---|---|---|---|---|---|---|---|
| score | category | timepoint | | (N = 15) | | (N = 7) | | (N = 22) | |
| UPDRS | Collected | Visit 2 Baseline | n | 15 | | 7 | | 22 | |
| Total | value | | Mean (SD) | 37.0 | (13.3) | 41.3 | (15.0) | 38.4 | (13.7) |
| Score | | | Median (Min, Max) | 36.0 | (17, 66) | 41.0 | (21, 63) | 37.5 | (17, 66) |
| | | Visit 6 Clinic | n | 15 | | 7 | | 22 | |
| | | Efficacy Visit | Mean (SD) | 30.5 | (13.1) | 37.7 | (12.5) | 32.8 | (13.1) |
| | | | Median (Min, Max) | 29.0 | (13, 54) | 41.0 | (22, 51) | 30.5 | (13, 54) |
| | | Visit 7 Clinic | n | 15 | | 7 | | 22 | |
| | | Efficacy Visit | Mean (SD) | 31.3 | (13.9) | 37.4 | (12.7) | 33.3 | (13.6) |
| | | | Median (Min, Max) | 30.0 | (15, 63) | 39.0 | (20, 51) | 31.0 | (15, 63) |
| | | Visit 9 Follow- | n | 15 | | 7 | | 22 | |
| | | up Clinic Visit | Mean (SD) | 32.6 | (12.5) | 34.0 | (14.3) | 33.0 | (12.8) |
| | | | Median (Min, Max) | 31.0 | (15, 56) | 34.0 | (11, 53) | 31.5 | (11, 56) |
| | Absolute | Visit 6 Clinic | n | 15 | | 7 | | 22 | |
| | change from | Efficacy Visit | Mean (SD) | −6.5 | (7.6) | −3.6 | (7.0) | −5.5 | (7.3) |
| | baseline | | Median (Min, Max) | −5.0 | (−23, 6) | −4.0 | (−12, 9) | −5.0 | (−23, 9) |
| | | | LMM^{a} p-value | 0.0005^{b} | | 0.2707^{c} | | 0.2589^{d} | |
| | | Visit 7 Clinic | n | 15 | | 7 | | 22 | |
| | | Efficacy Visit | Mean (SD) | −5.7 | (6.0) | −3.9 | (9.3) | −5.1 | (7.1) |
| | | | Median (Min, Max) | −5.0 | (−15, 4) | −6.0 | (−14, 15) | −5.5 | (−15, 15) |
| | | | LMM^{a} p-value | 0.0021^{b} | | 0.2282^{c} | | 0.4222^{d} | |
| | | Visit 9 Follow- | n | 15 | | 7 | | 22 | |
| | | up Clinic Visit | Mean (SD) | −4.4 | (9.0) | −7.3 | (5.4) | −5.3 | (8.0) |
| | | | Median (Min, Max) | −3.0 | (−20, 7) | −6.0 | (−17, −2) | −4.0 | (−20, 7) |
| | | | LMM^{a} p-value | 0.0140^{b} | | 0.0157^{c} | | 0.5336^{d} | |
| | Relative | Visit 6 Clinic | n | 15 | | 7 | | 22 | |
| | change from | Efficacy Visit | Mean (SD) | −17.4 | (19.3) | −6.68 | (16.6) | −14.0 | (18.8) |
| | baseline (%) | | Median (Min, Max) | −15.2 | (−64, 20) | −14.6 | (−22, 25) | −15.0 | (−64, 25) |

| Visit 7 Clinic | n | 15 | | 7 | | 22 | |
|---|---|---|---|---|---|---|---|
| Efficacy Visit | Mean (SD) | −15.7 | (16.6) | −7.06 | (22.9) | −13.0 | (18.7) |
| | Median (Min, Max) | −19.2 | (−42, 15) | −14.8 | (−23, 42) | −16.8 | (−42, 42) |
| Visit 9 Follow- | n | 15 | | 7 | | 22 | |
| up Clinic Visit | Mean (SD) | −9.57 | (23.6) | −19.2 | (16.0) | −12.6 | (21.6) |
| | Median (Min, Max) | −11.5 | (−56, 23) | −11.5 | (−48, −5.6) | −11.5 | (−56, 23) |

TABLE 15
UPDRS total scores for Part III (Per protocol analysis set)

| Calculated | Result | Assessment | | NLX-112 | | Placebo | | Total | |
|---|---|---|---|---|---|---|---|---|---|
| score | category | timepoint | | (N = 15) | | (N = 7) | | (N = 22) | |
| Part 3 | Collected | Visit 2 | n | 15 | | 7 | | 22 | |
| Motor | value | Baseline | Mean (SD) | 17.5 | (9.4) | 17.0 | (10.3) | 17.4 | (9.4) |
| Total | | | Median (Min, Max) | 17.0 | (3, 39) | 16.0 | (1, 33) | 16.5 | (1, 39) |
| | | Visit 6 Clinic | n | 15 | | 7 | | 22 | |
| | | Efficacy | Mean (SD) | 13.9 | (8.8) | 18.3 | (11.5) | 15.3 | (9.7) |
| | | Visit | Median (Min, Max) | 14.0 | (2, 30) | 24.0 | (0, 31) | 14.5 | (0, 31) |
| | | Visit 7 Clinic | n | 15 | | 7 | | 22 | |
| | | Efficacy | Mean (SD) | 13.9 | (8.5) | 17.1 | (10.5) | 14.9 | (9.1) |
| | | Visit | Median (Min, Max) | 14.0 | (1, 35) | 18.0 | (2, 32) | 14.5 | (1, 35) |
| | | Visit 9 | n | 15 | | 7 | | 22 | |
| | | Follow-up | Mean (SD) | 14.8 | (8.1) | 14.6 | (9.0) | 14.7 | (8.2) |
| | | Clinic Visit | Median (Min, Max) | 13.0 | (2, 32) | 17.0 | (0, 27) | 13.5 | (0, 32) |
| | Absolute | Visit 6 Clinic | n | 15 | | 7 | | 22 | |
| | change | Efficacy | Mean (SD) | −3.7 | (4.7) | 1.3 | (6.9) | −2.1 | (5.8) |
| | from | Visit | Median (Min, Max) | −4.0 | (−12, 5) | 0.0 | (−7, 15) | −2.0 | (−12, 15) |
| | baseline | | LMM^{a} p-value | 0.0072^{b} | | 0.5290^{c} | | 0.0405^{d} | |
| | | Visit 7 Clinic | n | 15 | | 7 | | 22 | |
| | | Efficacy | Mean (SD) | −3.7 | (4.6) | 0.1 | (8.2) | −2.5 | (6.1) |
| | | Visit | Median (Min, Max) | −4.0 | (−13, 3) | −1.0 | (−10, 16) | −3.0 | (−13, 16) |
| | | | LMM^{a} p-value | 0.0072^{b} | | 0.9728^{c} | | 0.1150^{d} | |
| | | Visit 9 | n | 15 | | 7 | | 22 | |
| | | Follow-up | Mean (SD) | −2.7 | (5.5) | −2.4 | (2.9) | −2.6 | (4.7) |
| | | Clinic Visit | Median (Min, Max) | −2.0 | (−15, 4) | −3.0 | (−6, 3) | −2.0 | (−15, 4) |
| | | | LMM^{a} p-value | 0.0429^{b} | | 0.1940^{c} | | 0.9344^{d} | |
| | Relative | Visit 6 Clinic | n | 15 | | 7 | | 22 | |
| | change | Efficacy Visit | Mean (SD) | −21.8 | (30.9) | −4.98 | (58.1) | −16.4 | (40.8) |
| | from | | Median (Min, Max) | −21.4 | (−75, 33) | 0.00 | (−100, 94) | −21.1 | (−100, 94) |
| | baseline | Visit 7 Clinic | n | 15 | | 7 | | 22 | |
| | (%) | Efficacy | Mean (SD) | −21.5 | (28.5) | 17.5 | (60.5) | −9.08 | (44.0) |
| | | Visit | Median (Min, Max) | −33.3 | (−67, 27) | −4.17 | (−50, 100) | −15.5 | (−67, 100) |

| Visit 9 | n | 15 | | 7 | | 22 | |
|---|---|---|---|---|---|---|---|
| Follow-up | Mean (SD) | −11.2 | (32.5) | −25.7 | (37.4) | −15.8 | (34.0) |
| Clinic Visit | Median (Min, Max) | −14.3 | (−65, 50) | −16.7 | (−100, 21) | −15.5 | (−100, 50) |

TABLE 16
UDysRS total score for all Parts (Full analysis set)

| Calculated | Result | Assessment | | NLX-112 | | Placebo | | Total | |
|---|---|---|---|---|---|---|---|---|---|
| score | category | timepoint | | (N = 18) | | (N = 9) | | (N = 27) | |
| UDysRS Parts | Collected | Visit 2 Baseline | n | 18 | | 9 | | 27 | |
| 1, 2, 3, and 4 | value | | Mean (SD) | 31.8 | (8.9) | 43.7 | (14.8) | 35.7 | (12.3) |
| most severe | | | Median (Min, Max) | 31.0 | (13, 47) | 42.0 | (27, 66) | 33.0 | (13, 66) |
| | | Visit 6 Clinic | n | 15 | | 8 | | 23 | |
| | | Efficacy Visit | Mean (SD) | 27.5 | (11.2) | 37.6 | (13.4) | 31.0 | (12.7) |
| | | | Median (Min, Max) | 28.0 | (14, 55) | 35.5 | (21, 66) | 29.0 | (14, 66) |
| | | Visit 7 Clinic | n | 15 | | 8 | | 23 | |
| | | Efficacy Visit | Mean (SD) | 25.3 | (10.2) | 36.8 | (12.0) | 29.3 | (12.0) |
| | | | Median (Min, Max) | 24.0 | (11, 51) | 35.0 | (22, 57) | 28.0 | (11, 57) |
| | Absolute | Visit 6 Clinic | n | 15 | | 8 | | 23 | |
| | change from | Efficacy Visit | Mean (SD) | −4.1 | (9.3) | −3.5 | (6.2) | −3.9 | (8.2) |
| | baseline | | Median (Min, Max) | −2.0 | (−21, 11) | −3.5 | (−14, 6) | −2.0 | (−21, 11) |
| | | | LMM^{a} p-value | 0.0235^{b} | | 0.5122^{c} | | 0.4148^{d} | |
| | | Visit 7 Clinic | n | 15 | | 8 | | 23 | |
| | | Efficacy Visit | Mean (SD) | −6.3 | (7.0) | −4.4 | (10.5) | −5.7 | (8.2) |
| | | | Median (Min, Max) | −8.0 | (−19, 7) | −4.5 | (−18, 16) | −6.0 | (−19, 16) |
| | | | LMM^{a} p-value | 0.0014^{b} | | 0.3411^{c} | | 0.2346^{d} | |
| | Relative | Visit 6 Clinic | n | 15 | | 8 | | 23 | |
| | change from | Efficacy Visit | Mean (SD) | −10.2 | (28.0) | −7.93 | (15.1) | −9.43 | (23.9) |
| | baseline | | Median (Min, Max) | −6.45 | (−51, 38) | −8.85 | (−28, 18) | −6.45 | (−51, 38) |

| | | | | | |
|---|---|---|---|---|---|
| (%) | Visit 7 Clinic | n | 15 | 8 | 23 |

| | | | | | | | |
|---|---|---|---|---|---|---|---|
| Efficacy Visit | Mean (SD) | −19.0 | (22.6) | −7.99 | (26.2) | −15.2 | (23.9) |
| | Median (Min, Max) | −25.8 | (−50, 31) | −14.2 | (−36, 48) | −18.5 | (−50, 48) |

TABLE 17
UDysRS total score for Part III and IV - Most severe assessment
session and highest questionnaire item score (Full analysis set)

| Calculated | Result | Assessment | | NLX-112 | | Placebo | | Total | |
|---|---|---|---|---|---|---|---|---|---|
| score | category | timepoint | | (N = 18) | | (N = 9) | | (N = 27) | |
| UDysRS Parts | Collected | Visit 2 Baseline | n | 18 | | 9 | | 27 | |
| 3 and 4 most | value | | Mean (SD) | 15.3 | (5.8) | 20.1 | (7.9) | 16.9 | (6.8) |
| severe | | | Median (Min, Max) | 13.5 | (6, 29) | 19.0 | (11, 34) | 17.0 | (6, 34) |
| | | Visit 6 Clinic | n | 15 | | 8 | | 23 | |
| | | Efficacy Visit | Mean (SD) | 13.8 | (8.5) | 17.4 | (6.7) | 15.0 | (8.0) |
| | | | Median (Min, Max) | 12.0 | (2, 36) | 17.5 | (10, 31) | 13.0 | (2, 36) |
| | | Visit 7 Clinic | n | 15 | | 8 | | 23 | |
| | | Efficacy Visit | Mean (SD) | 12.4 | (7.2) | 17.6 | (7.2) | 14.2 | (7.5) |
| | | | Median (Min, Max) | 11.0 | (5, 31) | 15.5 | (10, 32) | 13.0 | (5, 32) |
| | Absolute | Visit 6 Clinic | n | 15 | | 8 | | 23 | |
| | change from | Efficacy Visit | Mean (SD) | −1.7 | (4.3) | −1.9 | (3.0) | −1.8 | (3.8) |
| | baseline | | Median (Min, Max) | −1.0 | (−10, 7) | −2.0 | (−8, 2) | −2.0 | (−10, 7) |
| | | | LMM^{a} p-value | 0.1071^{b} | | 0.2826^{c} | | 0.9314^{d} | |
| | | Visit 7 Clinic | n | 15 | | 8 | | 23 | |
| | | Efficacy Visit | Mean (SD) | −3.1 | (4.2) | −1.6 | (5.5) | −2.6 | (4.6) |
| | | | Median (Min, Max) | −4.0 | (−11, 3) | −1.5 | (−12, 7) | −2.0 | (−12, 7) |
| | | | LMM^{a} p-value | 0.0060^{b} | | 0.3598^{c} | | 0.3488^{d} | |
| | Relative | Visit 6 Clinic | n | 15 | | 8 | | 23 | |
| | change from | Efficacy Visit | Mean (SD) | −15.8 | (26.9) | −7.81 | (14.5) | −13.0 | (23.3) |
| | baseline | | Median (Min, Max) | −8.33 | (−67, 24) | −9.17 | (−30, 18) | −8.82 | (−67, 24) |

| | | | | | |
|---|---|---|---|---|---|
| (%) | Visit 7 Clinic | n | 15 | 8 | 23 |

| | | | | | | | |
|---|---|---|---|---|---|---|---|
| Efficacy Visit | Mean (SD) | −21.0 | (24.5) | −4.94 | (25.7) | −15.4 | (25.6) |
| | Median (Min, Max) | −31.6 | (−55, 17) | −5.57 | (−44, 41) | −11.8 | (−55, 41) |

TABLE 18
UDysRS total score for Part III and IV - Average of assessment sessions
and highest questionnaire item score (Full analysis set)

| Calculated | Result | Assessment | | NLX-112 | | Placebo | | Total | |
|---|---|---|---|---|---|---|---|---|---|
| score | category | timepoint | | (N = 18) | | (N = 9) | | (N = 27) | |
| UDysRS | Collected | Visit 2 | n | 18 | | 9 | | 27 | |
| Parts 3 | value | Baseline | Mean (SD) | 13.98 | (6.09) | 19.19 | (7.42) | 15.72 | (6.89) |
| and 4 | | | Median (Min, Max) | 13.17 | (3.7, 27.0) | 17.33 | (11.0, 32.7) | 13.33 | (3.7, 32.7) |
| average | | Visit 6 Clinic | n | 15 | | 8 | | 23 | |
| | | Efficacy Visit | Mean (SD) | 11.60 | (7.48) | 16.46 | (5.85) | 13.29 | (7.22) |
| | | | Median (Min, Max) | 10.33 | (0.7, 30.0) | 15.67 | (10.3, 28.7) | 11.67 | (0.7, 30.0) |
| | | Visit 7 Clinic | n | 15 | | 8 | | 23 | |
| | | Efficacy Visit | Mean (SD) | 10.78 | (6.88) | 15.83 | (7.22) | 12.54 | (7.26) |
| | | | Median (Min, Max) | 9.67 | (2.0, 27.7) | 13.33 | (9.3, 30.7) | 11.67 | (2.0, 30.7) |
| | Absolute | Visit 6 Clinic | n | 15 | | 8 | | 23 | |
| | change from | Efficacy Visit | Mean (SD) | −2.49 | (4.22) | −1.96 | (3.09) | −2.30 | (3.80) |
| | baseline | | Median (Min, Max) | −1.67 | (−12.0, 3.0) | −1.00 | (−8.3, 0.7) | −1.33 | (−12.0, 3.0) |
| | | | LMM^{a} p-value | 0.0107^{b} | | 0.2866^{c} | | 0.4952^{d} | |
| | | Visit 7 Clinic | n | 15 | | 8 | | 23 | |
| | | Efficacy Visit | Mean (SD) | −3.31 | (3.38) | −2.58 | (5.35) | −3.06 | (4.06) |
| | | | Median (Min, Max) | −3.33 | (−9.3, 1.0) | −1.50 | (−14.0, 4.7) | −2.33 | (−14.0, 4.7) |
| | | | LMM^{a} p-value | 0.0012^{b} | | 0.1350^{c} | | 0.4280^{d} | |
| | Relative | Visit 6 Clinic | n | 15 | | 8 | | 23 | |
| | change from | Efficacy Visit | Mean (SD) | −17.3 | (35.6) | −8.07 | (13.1) | −14.1 | (29.7) |
| | baseline | | Median (Min, Max) | −20.0 | (−88, 64) | −7.41 | (−32, 6.1) | −11.4 | (−88, 64) |

| | | | | | |
|---|---|---|---|---|---|
| (%) | Visit 7 Clinic | n | 15 | 8 | 23 |

| | | | | | | | |
|---|---|---|---|---|---|---|---|
| Efficacy Visit | Mean (SD) | −25.3 | (23.3) | −11.8 | (23.3) | −20.6 | (23.7) |
| | Median (Min, Max) | −31.5 | (−63, 9.1) | −7.61 | (−55, 27) | −20.0 | (−63, 27) |

TABLE 19
UDysRS total score for all Parts - Average of assessment sessions Part 3 and Part 4 (Full analysis set)

| Calculated | Result | Assessment | | NLX-112 | | Placebo | | Total | |
|---|---|---|---|---|---|---|---|---|---|
| score | category | timepoint | | (N = 18) | | (N = 9) | | (N = 27) | |
| UDysRS | Collected | Visit 2 | n | 18 | | 9 | | 27 | |
| Parts 1, 2, | value | Baseline | Mean (SD) | 30.43 | (9.62) | 42.74 | (14.34) | 34.53 | (12.60) |
| 3, and 4 | | | Median (Min, Max) | 29.67 | (10.7, 46.0) | 41.33 | (26.7, 64.7) | 32.33 | (10.7, 64.7) |
| average | | Visit 6 Clinic | n | 15 | | 8 | | 23 | |
| | | Efficacy Visit | Mean (SD) | 25.33 | (10.11) | 36.71 | (12.91) | 29.29 | (12.20) |
| | | | Median (Min, Max) | 25.33 | (12.7, 49.0) | 35.50 | (21.3, 63.7) | 26.00 | (12.7, 63.7) |
| | | Visit 7 Clinic | n | 15 | | 8 | | 23 | |
| | | Efficacy Visit | Mean (SD) | 23.64 | (9.96) | 34.96 | (11.79) | 27.58 | (11.73) |
| | | | Median (Min, Max) | 23.00 | (10.3, 47.7) | 33.67 | (21.0, 55.7) | 26.67 | (10.3, 55.7) |
| | Absolute | Visit 6 Clinic | n | 15 | | 8 | | 23 | |
| | change | Efficacy Visit | Mean (SD) | −4.82 | (9.32) | −3.58 | (5.45) | −4.39 | (8.07) |
| | from | | Median (Min, Max) | −3.00 | (−21.0, 7.3) | −3.50 | (−14.3, 4.0) | −3.00 | (−21.0, 7.3) |
| | baseline | | LMM^{a} p-value | 0.0046^{b} | | 0.5673^{c} | | 0.2157^{d} | |
| | | Visit 7 Clinic | n | 15 | | 8 | | 23 | |
| | | Efficacy Visit | Mean (SD) | −6.51 | (6.81) | −5.33 | (10.23) | −6.10 | (7.95) |
| | | | Median (Min, Max) | −7.67 | (−19.3, 5.7) | −5.17 | (−20.0, 13.7) | −6.00 | (−20.0, 13.7) |
| | | | LMM^{a} p-value | 0.0004^{b} | | 0.2304^{c} | | 0.2221^{d} | |
| | Relative | Visit 6 Clinic | n | 15 | | 8 | | 23 | |
| | change | Efficacy Visit | Mean (SD) | −9.33 | (35.5) | −8.55 | (13.0) | −9.06 | (29.2) |
| | from | | Median (Min, Max) | −10.6 | (−57, 69) | −9.97 | (−29, 12) | −10.6 | (−57, 69) |
| | baseline | Visit 7 Clinic | n | 15 | | 8 | | 23 | |
| | (%) | Efficacy Visit | Mean (SD) | −19.5 | (22.3) | −10.9 | (25.2) | −16.5 | (23.2) |
| | | | Median (Min, Max) | −21.9 | (−51, 25) | −15.1 | (−41, 41) | −20.5 | (−51, 41) |

TABLE 20
UPDRS total scores for all Parts (Full analysis set)

| Calculated | Result | Assessment | | NLX-112 | | Placebo | | Total | |
|---|---|---|---|---|---|---|---|---|---|
| score | category | timepoint | | (N = 18) | | (N = 9) | | (N = 27) | |
| UPDRS | Collected | Visit 2 | n | 18 | | 9 | | 27 | |
| Total | value | Baseline | Mean (SD) | 38.3 | (14.9) | 42.4 | (13.2) | 39.7 | (14.3) |
| Score | | | Median (Min, Max) | 37.5 | (17, 69) | 45.0 | (21, 63) | 41.0 | (17, 69) |
| | | Visit 6 Clinic | n | 15 | | 8 | | 23 | |
| | | Efficacy | Mean (SD) | 30.5 | (13.1) | 37.8 | (11.6) | 33.0 | (12.9) |
| | | Visit | Median (Min, Max) | 29.0 | (13, 54) | 39.5 | (22, 51) | 32.0 | (13, 54) |
| | | Visit 7 Clinic | n | 15 | | 8 | | 23 | |
| | | Efficacy | Mean (SD) | 31.3 | (13.9) | 38.8 | (12.3) | 33.9 | (13.6) |
| | | Visit | Median (Min, Max) | 30.0 | (15, 63) | 42.5 | (20, 51) | 32.0 | (15, 63) |
| | | Visit 9 | n | 15 | | 8 | | 23 | |
| | | Follow-up | Mean (SD) | 32.6 | (12.5) | 35.6 | (14.0) | 33.7 | (12.8) |
| | | Clinic Visit | Median (Min, Max) | 31.0 | (15, 56) | 36.0 | (11, 53) | 32.0 | (11, 56) |
| | Absolute | Visit 6 Clinic | n | 15 | | 8 | | 23 | |
| | change from | Efficacy | Mean (SD) | −6.5 | (7.6) | −4.4 | (6.8) | −5.7 | (7.2) |
| | baseline | Visit | Median (Min, Max) | −5.0 | (−23, 6) | −5.5 | (−12, 9) | −5.0 | (−23, 9) |
| | | | LMM^{a} p-value | 0.0004^{b} | | 0.1411^{c} | | 0.3270^{d} | |
| | | Visit 7 Clinic | n | 15 | | 8 | | 23 | |
| | | Efficacy | Mean (SD) | −5.7 | (6.0) | −3.4 | (8.7) | 4.9 | (7.0) |
| | | Visit | Median (Min, Max) | −5.0 | (−15, 4) | −5.5 | (−14, 15) | −5.0 | (−15, 15) |
| | | | LMM^{a} p-value | 0.0017^{b} | | 0.2792^{c} | | 0.2967^{d} | |
| | | Visit 9 | n | 15 | | 8 | | 23 | |
| | | Follow-up | Mean (SD) | −4.4 | (9.0) | −6.5 | (5.5) | −5.1 | (7.9) |
| | | Clinic Visit | Median (Min, Max) | −3.0 | (−20, 7) | −4.5 | (−17, −1) | −3.0 | (−20, 7) |
| | | | LMM^{a} p-value | 0.0118^{b} | | 0.0226^{c} | | 0.7200^{d} | |
| | Relative | Visit 6 Clinic | n | 15 | | 8 | | 23 | |
| | change from | Efficacy | Mean (SD) | −17.4 | (19.3) | −8.45 | (16.2) | −14.3 | (18.5) |
| | baseline (%) | Visit | Median (Min, Max) | −15.2 | (−64, 20) | −15.0 | (−22, 25) | −15.2 | (−64, 25) |

| Visit 7 Clinic | n | 15 | | 8 | | 23 | |
|---|---|---|---|---|---|---|---|
| Efficacy | Mean (SD) | −15.7 | (16.6) | −6.17 | (21.3) | −12.4 | (18.5) |
| Visit | Median (Min, Max) | −19.2 | (−42, 15) | −13.5 | (−23, 42) | −14.8 | (−42, 42) |
| Visit 9 | n | 15 | | 8 | | 23 | |
| Follow-up | Mean (SD) | −9.57 | (23.6) | −17.1 | (16.0) | −12.2 | (21.2) |
| Clinic Visit | Median (Min, Max) | −11.5 | (−56, 23) | −11.3 | (−48, −2.1) | −11.5 | (−56, 23) |

TABLE 21
UPDRS total scores for Part III (Full analysis set)

| Calculated | Result | Assessment | | NLX-112 | | Placebo | | Total | |
|---|---|---|---|---|---|---|---|---|---|
| score | category | timepoint | | (N = 18) | | (N = 9) | | (N = 27) | |
| Part 3 | Collected | Visit 2 | n | 18 | | 9 | | 27 | |
| Motor | value | Baseline | Mean (SD) | 17.8 | (10.3) | 16.7 | (9.0) | 17.4 | (9.8) |
| Total | | | Median (Min, Max) | 16.0 | (3, 39) | 16.0 | (1, 33) | 16.0 | (1, 39) |
| | | Visit 6 Clinic | n | 15 | | 8 | | 23 | |
| | | Efficacy Visit | Mean (SD) | 13.9 | (8.8) | 17.5 | (10.9) | 15.1 | (9.5) |
| | | | Median (Min, Max) | 14.0 | (2, 30) | 19.0 | (0, 31) | 14.0 | (0, 31) |
| | | Visit 7 Clinic | n | 15 | | 8 | | 23 | |
| | | Efficacy Visit | Mean (SD) | 13.9 | (8.5) | 17.8 | (9.9) | 15.2 | (9.0) |
| | | | Median (Min, Max) | 14.0 | (1, 35) | 20.0 | (2, 32) | 15.0 | (1, 35) |
| | | Visit 9 | n | 15 | | 8 | | 23 | |
| | | Follow-up | Mean (SD) | 14.8 | (8.1) | 15.5 | (8.8) | 15.0 | (8.1) |
| | | Clinic Visit | Median (Min, Max) | 13.0 | (2, 32) | 17.5 | (0, 27) | 14.0 | (0, 32) |
| | Absolute | Visit 6 Clinic | n | 15 | | 8 | | 23 | |
| | change | Efficacy Visit | Mean (SD) | −3.7 | (4.7) | 0.4 | (6.9) | −2.3 | (5.7) |
| | from | | Median (Min, Max) | −4.0 | (−12, 5) | −0.5 | (−7, 15) | −2.0 | (−12, 15) |
| | baseline | | LMM^{a} p-value | 0.0073^{b} | | 0.8599^{c} | | 0.0803^{d} | |
| | | Visit 7 Clinic | n | 15 | | 8 | | 23 | |
| | | Efficacy Visit | Mean (SD) | −3.7 | (4.6) | 0.6 | (7.7) | −2.2 | (6.1) |
| | | | Median (Min, Max) | −4.0 | (−13, 3) | 0.0 | (−10, 16) | −3.0 | (−13, 16) |
| | | | LMM^{a} p-value | 0.0073^{b} | | 0.7527^{c} | | 0.0633^{d} | |
| | | Visit 9 | n | 15 | | 8 | | 23 | |
| | | Follow-up | Mean (SD) | −2.7 | (5.5) | −1.6 | (3.5) | −2.3 | (4.8) |
| | | Clinic Visit | Median (Min, Max) | −2.0 | (−15, 4) | −2.5 | (−6, 4) | −2.0 | (−15, 4) |
| | | | LMM^{a} p-value | 0.0435^{b} | | 0.3527^{c} | | 0.6476^{d} | |
| | Relative | Visit 6 Clinic | n | 15 | | 8 | | 23 | |
| | change | Efficacy Visit | Mean (SD) | −21.8 | (30.9) | −8.52 | (54.7) | −17.2 | (40.0) |
| | from | | Median (Min, Max) | −21.4 | (−75, 33) | −10.6 | (−100, 94) | −21.2 | (−100, 94) |
| | baseline | Visit 7 Clinic | n | 15 | | 8 | | 23 | |
| | (%) | Efficacy Visit | Mean (SD) | −21.5 | (28.5) | 18.1 | (56.0) | −7.72 | (43.5) |
| | | | Median (Min, Max) | −33.3 | (−67, 27) | 8.63 | (−50, 100) | −14.3 | (−67, 100) |

| Visit 9 | n | 15 | | 8 | | 23 | |
|---|---|---|---|---|---|---|---|
| Follow-up | Mean (SD) | −11.2 | (32.5) | −19.7 | (38.5) | −14.1 | (34.1) |
| Clinic Visit | Median (Min, Max) | −14.3 | (−65, 50) | −15.5 | (−100, 22) | −14.3 | (−100, 50) |

TABLE 22
Effects of NLX-112 and Placebo on LID and Parkinson's symptoms

| NLX-112 (n = 15) | | | Placebo (n = 7) | | |
|---|---|---|---|---|---|
| Baseline | Δ D 28 | Δ D 42 | Baseline | Δ D 28 | Δ D 42 |

UDysRS - with highest score of assessment sessions

| | | | | | | | | | | | | |
|---|---|---|---|---|---|---|---|---|---|---|---|---|
| Total (Parts 1 to 4) | 31.6 | (9.7) | −4.1 | (9.3)* | −6.3 | (7.0)** | 39.9 | (14.2) | −2.0 | (4.9) | −2.4 | (9.7) |
| Parts 3 + 4 | 15.5 | (6.2) | −1.7 | (4.3) | −3.1 | (4.2)** | 18.1 | (7.9) | −1.0 | (1.8) | −0.1 | (3.8) |
| Part 3 sum of items | 24.7 | (14.9) | −5.5 | (10.4)** | −7.8 | (11.1)*** | 28.9 | (22.0) | −3.0 | (3.5) | −1.7 | (5.4) |

UDysRS - with average of assessment sessions

| | | | | | | | | | | | | |
|---|---|---|---|---|---|---|---|---|---|---|---|---|
| Total (Parts 1 to 4) | 30.1 | (10.5) | −4.8 | (9.3)** | −6.5 | (6.8)*** | 39.1 | (13.8) | −2.1 | (3.6) | −3.2 | (9.0) |
| Parts 3 + 4 | 14.1 | (6.6) | −2.5 | (4.2)** | −3.3 | (3.4)*** | 17.4 | (7.5) | −1.1 | (1.9) | −1.0 | (2.9) |
| Part 3 sum of items | 21.5 | (14.4) | −5.5 | (9.3)***# | −7.1 | (8.8)****# | 26.8 | (20.5) | −2.29 | (5.2) | −3.2 | (4.4) |

UPDRS

| | | | | | | | | | | | | |
|---|---|---|---|---|---|---|---|---|---|---|---|---|
| Total (Parts 1 to 4) | 37.0 | (13.3) | −6.5 | (7.6)*** | −5.7 | (6.0)** | 41.3 | (15.0) | −3.6 | (7.0) | −3.9 | (9.3) |
| Part 1 | 1.0 | (1.0) | 0.2 | (1.5) | 0.1 | (1.2) | 1.6 | (1.5) | −0.3 | (1.4) | 0.3 | (2.0) |
| Part 2 | 10.7 | (4.4) | −2.1 | (4.3)* | −1.2 | (3.7) | 12.9 | (4.7) | −1.9 | (3.2) | −3.1 | (2.0)^{Δ} |
| Part 3 | 17.5 | (9.4) | −3.7 | (4.7)**# | −3.7 | (4.6)** | 17.0 | (10.3) | 1.3 | (6.9) | 0.1 | (8.2) |
| Part 4 | 8.0 | (2.0) | −0.9 | (1.8)* | −0.9 | (2.5)* | 8.4 | (2.0) | −2.7 | (1.0)* | −1.1 | (2.3) |

Results are mean (SD) of the per-protocol set for Baseline and change from baseline values at days 28 (end of up-titration period) and 42 (end of stable dosing period).
Numbers in bold are significantly different to baseline:
^{Δ}p = 0.05,
*p < 0.05,
**p < 0.01,
***p < 0.001,
****p < 0.0001.
#p < 0.05 significant difference to placebo.
Linear mixed model (LMM).

TABLE 23
Effects of NLX-112 on measures of motor symptoms
and CGI-C in patients treated with amantadine

| | | Amantadine | UDysRS | | UDysRS | | UPDRS | | UPDRS Part | | |
|---|---|---|---|---|---|---|---|---|---|---|---|
| | | Treatment | Total | | Part 3 + 4 | | Total | | 3 | | |
| | Sex/ | Duration | Base- | | Base- | | Base- | | Base- | | |
| Patient | Age | (Years) | line | Δ | line | Δ | line | Δ | line | Δ | CGI-C |
| A | F/51 | 1 | 18 | −6 | 11 | −6 | 22 | −5 | 8 | −4 | No |
| | | | | | | | | | | | change |
| B | M/69 | 2 | 43 | −2 | 22 | +3 | 44 | −15 | 22 | −9 | No |
| | | | | | | | | | | | change |
| C | F/75 | 7 | 44 | +7 | 29 | +2 | 51 | −11 | 28 | −13 | Minimally |
| | | | | | | | | | | | worse |
| D | F/49 | 11 | 39 | −11 | 28 | −11 | 26 | −5 | 11 | +3 | Very |
| | | | | | | | | | | | much |
| | | | | | | | | | | | improved |
| E | F/72 | 6 | 31 | −8 | 17 | −6 | 30 | +3 | 15 | +2 | Minimally |
| | | | | | | | | | | | improved |

| | | | | |
|---|---|---|---|---|
| Mean change from baseline | −4.0 | −4.4 | −6.6 | −4.2 |

Patients on NLX-112 numerically rated their overall PD symptoms as more improved than patients on placebo, but there were no statistically significant differences between the treatment groups (Table 24). The proportion of patients in the NLX-112 group who rated their condition as minimally improved, much improved or very much improved was 33% and 53% on days 28 and 42, respectively, compared to 29% in the placebo group on both days.

TABLE 24
Effects of NLX-112 and Placebo on CGI-C

| | NLX-112 (n = 15) | | | Placebo (n = 7) | |
|---|---|---|---|---|---|
| | Up-Titration | Stable Dosing | | Up-Titration | Stable Dosing |
| CGI-C | (d 28) | (d 42) | | (d 28) | (d 42) |
| Total Improved | 5 (33%) | 8 | (53%) | 2 (29%) | 2 (29%) |
| Very much improved | 0 | 1 | (6.7%) | 0 | 0 |
| Much improved | 2 (13%) | 4 | (27%) | 2 (29%) | 1 (14%) |
| Minimally improved | 3 (20%) | 3 | (20%) | 0 | 1 (14%) |
| No change | 9 (60%) | 6 | (40%) | 4 (57%) | 5 (71%) |
| Minimally worse | 1 (6.7%) | 1 | (6.7%) | 1 (14%) | 0 |

The results on the exploratory objectives are provided in Table 25. Treatment with both NLX-112 and placebo resulted in a statistically significant improvement in the patients' reported pain, based on the change from baseline in the KPPS total score on Day 42. There were no statistically significant differences between the NLX-112 group and the placebo group in terms of change from baseline to Day 28 or Day 42.

NLX-112 had no consistent statistically significant effect on the patients' quality of life (based on PDQ39 score), anxiety and depression (HADS), incontinence (ICIQ-OAB) or sleepiness (ESS) on Days 28 or 42. For ICIQ-OAB, a statistically significant increase (impairment) in ICIQ-OAB total score was shown in the placebo group on Day 42, but not on Day 28.

TABLE 25
Effects of NLX-112 and Placebo on measures of non-motor symptoms (Per Protocol Set)

| | NLX-112 (n = 15) | | | | | | Placebo (n = 7) | | | | | |
|---|---|---|---|---|---|---|---|---|---|---|---|---|
| | Baseline | | Δ D 28 | | Δ D 42 | | Baseline | | Δ D 28 | | Δ D 42 | |
| ESS | 8.3 | (5.2) | −1.1 | (3.3) | −0.9 | (3.4) | 9.0 | (4.7) | 2.3 | (3.7) | 1.4 | (4.2) |
| HADS (Anxiety) | 4.5 | (2.5) | −0.5 | (2.5) | −−0.3 | (3.6) | 4.4 | (3.4) | −0.1 | (1.7) | 1.0 | (2.8) |
| HADS (Depression) | 3.7 | (2.7) | −0.4 | (1.7) | −0.6 | (1.9) | 3.0 | (1.8) | 0.0 | (2.4) | 0.4 | (2.4) |
| ICIQ-OAB | 18.3 | (11.7) | 0.7 | (6.5) | −0.8 | (8.02) | 14.3 | (14.7) | 4.1 | (5.4) | 6.9 | (9.2)* |
| KPPS | 16.0 | (9.9) | −4.6 | (7.5)** | −4.8 | (7.8)** | 24.0 | (20.9) | −5.9 | (6.8) | −6.9 | (6.7)* |
| PDQ39 | 18.7 | (10.2) | −1.5 | (8.0) | −0.6 | (6.5) | 23.5 | (11.8) | −1.7 | (5.1) | −1.3 | (7.7) |

Results are mean (S.D.) of the per-protocol set for Baseline values and change from baseline values at days 28 (end of up-titration period) and 42 (end of stable dosing period).
Numbers in bold are significantly different to baseline:
*p < 0.05,
**p < 0.01.
Linear mixed model (LMM). ESS: The Epworth Sleepiness Scale; HADS: The Hospital Anxiety Depression Scale; ICIQ-OAB: International Consultation on Incontinence Questionnaire - Overactive Bladder Module; KPPS: King's Parkinson's disease Pain Scale; PDQ39: The Parkinson's Disease Questionnaire.

Based on time recorded in the PD Home Dyskinesia eDiary, the proportion of ‘good ON time’ experienced by patients at baseline was 57.6% in the NLX-112 group (n=15) and 54.8% in the placebo group (n=7). Mean absolute change from baseline in good ON time was +5.7% (n=13) and +5.2% (n=12) in the NLX-112 group, compared to +2.5% (n=6) and +3.4% (n=6) in the placebo group on days 28 and 42, respectively. The results show that NLX-112 elicited a numerical increase in good ON time more than placebo but there were no statistically significant changes from baseline to Day 28 or Day 42 within or between the treatment groups. Moreover, compliance was lowered on days 28 and 42 so these observations should be interpreted with caution.

### SUMMARY

The present study was the first to examine the effects of NLX-112 in patients with PD. Serotonin mechanisms have long been understood to be implicated in motor fluctuations in PD but suitable drug candidates have been elusive. Buspirone, sarizotan or eltoprazine have been investigated as anti-LID treatments, but they exhibit limited efficacy and/or interfere with the therapeutic effects of L-DOPA (Bara-Jimenez W et al., “Effects of serotonin 5-HT1A agonist in advanced Parkinson's disease,” Movement Disorders 2005, 20, 932-6; Politis M et al., “Serotonergic mechanisms responsible for levodopa-induced dyskinesias in Parkinson's disease patients,” The Journal of Clinical Investigation 2014, 124, 1340-9; Svenningsson P et al., “Eltoprazine counteracts 1-DOPA-induced dyskinesias in Parkinson's disease: a dose-finding study,” Brain, 2015, 138 (Pt 4), 963-973), possibly due to interaction with off-target sites (notably antagonism of dopamine receptors) and/or insufficient agonist efficacy at 5-HT_{1A} receptors. NLX-112 (a.k.a. befiradol or F13640) has exceptional selectivity for 5-HT_{1A} receptors and full agonist properties (Newman-Tancredi A et al., “Distinctive in vitro signal transduction profile of NLX-112, a potent and efficacious serotonin 5-HT1A receptor agonist,” J Pharm Pharmacol 2017, 69, 1178-1190), making it an attractive drug to explore the therapeutic potential of targeting 5-HT_{1A} receptors in PD patients.

The key findings of the present Phase 2A, randomized, double-blind, placebo-controlled study are that NLX-112 (i) was safe and generally well-tolerated in PD patients with troublesome LID; (ii) significantly reduced LID as assessed by the UDysRS; (iii) significantly reduced PD symptoms as assessed by UPDRS, including reductions in motor disability (UPDRS Part 3); (iv) improved CGI-C scores more than placebo-treated patients; and (v) also reduced LID and PD scores in the sub-group of patients that were concurrently treated with amantadine.

Baseline characteristics of the randomized patients (18 in the NLX-112 group and 9 in the placebo group) were generally comparable across the treatment groups (Table 3). Although three patients on NLX-112 withdrew from the study during the up-titration period, only one of them did so partially due to AEs (on and off phenomenon, vomiting and stress). Overall, NLX-112 was safe and well tolerated by most patients in the study and no SAEs occurred in the NLX-112 group. All AEs reported by patients on NLX-112 were mild to moderate in intensity. AEs were more frequently reported during the up-titration period in both treatment groups (Table 9). Nausea, dizziness, headache and insomnia, known as the most common TEAEs from previous studies on NLX-112, were also among the most common AEs reported by patients on NLX-112 in the present study (see Table 7). Dizziness and headache were also reported by patients in the placebo group, while nausea and insomnia were not. In line with the favorable AE profile, there were no clinically significant changes from baseline in ECG, vital signs, safety laboratory parameters or physical examinations in either the NLX-112 or placebo groups. No increase in suicidal ideation was observed in any patient. Overall, the safety profile did not differ between the NLX-112 group and the placebo group.

It should be noted that the present study used a slow up-titration because NLX-112 is rapidly absorbed into the CNS and can elicit Cmax related AEs, notably dizziness, headache and nausea. An alternative strategy is to use a slow-release formulation, as shown in a previous study in healthy volunteers (WO 2016/005527).

For efficacy measures, an L-DOPA challenge (150% of the patients' regular L-DOPA dose) was administered at baseline and Days 28 and 42 to overcome some of the variability in LID symptoms that can occur from day to day in PD patients. This is particularly useful in the context of exploratory trials with modest numbers of subjects (Svenningsson et al., 2015; Svenningsson P et al., “Safety and tolerability of IRL790 in Parkinson's disease with levodopa-induced dyskinesia-a phase 1b trial,” NPJ Parkinsons Dis 2018, 4, 35). Here, a highly statistically significant reduction in LID compared to baseline was observed in the NLX-112 group on days 28 and 42 based on UDysRS total score (Table 22, FIG. 1). This was the case whether the analyses were carried out based on the most severe of the three assessments (30, 60 or 90 min after L-DOPA challenge) or based on the average of the three measures. Moreover, NLX-112 also elicited significant reductions in the sum of the item scores from Part 3 of the UDysRS (objective evaluation of dyskinesia impairment) compared to the placebo group. The decrease in UDysRS scores in the NLX-112 group were significant despite the fact that baseline scores were lower than for the placebo group (meaning that there was less space for reduction), and that the study protocol included only a short (2-week) stable dosing period on the maximum dose. Interestingly, there was a further numerical reduction of UDysRS scores at Day 42 compared to Day 28, suggesting that there might be a time-dependent effect which could accentuate with longer treatment durations. A hint of improvement in ‘good ON time’ (i.e., ON without troublesome dyskinesia) was obtained from eDiary measures: patients on NLX-112 showed numerical greater increases than patients on placebo, but compliance was poor, so this observation should be interpreted with caution.

Amantadine, a NMDA receptor antagonist with additional pharmacological activities (Rascol O et al, “Amantadine in the treatment of Parkinson's disease and other movement disorders,” Lancet Neurol 2021, 20, 1048-1056), is recommended for the treatment of LIDs, but many patients either do not respond, show only marginal response (Sawada H et al., “Amantadine for dyskinesias in Parkinson's disease: a randomized controlled trial,” PLOS One 2010, 5, e15298) or experience loss of response over time (Thomas A et al., “Duration of amantadine benefit on dyskinesia of severe Parkinson's disease,” J Neurol Neurosurg Psychiatry 2004, 75, 141-3). Moreover, amantadine is associated with unwanted side-effects such as hallucinations and confusion as well as cardiovascular and gastrointestinal effects that can limit its utility (Pahwa R et al., “Amantadine extended release for levodopa-induced dyskinesia in Parkinson's disease (EASED Study),” Mov Disord 2015, 30, 788-95; Tanner C M et al., “EASE LID 2: A 2-Year Open-Label Trial of Gocovri (Amantadine) Extended Release for Dyskinesia in Parkinson's Disease,” J Parkinsons Dis 2020, 10, 543-558). The present study allowed inclusion of amantadine-treated patients, on the rationale that it reflects a ‘real world’ treatment situation and could provide early information on efficacy and tolerance of patients co-treated with both NLX-112 and amantadine. In fact, the study randomized 6 patients who were treated with amantadine prior to, and during, the study. One of the patients was in the placebo group (but dropped out due to deep brain stimulation surgery) whereas the other five were in the NLX-112 group and all successfully completed the trial. Notably, 3 of the 5 amantadine-using patients on NLX-112, showed numerical reductions in the UDysRS total score and UDysRS total objective score (Parts 3+4; see Table 23). These observations suggest that (i) NLX-112 may provide additional benefit over amantadine for reducing LID, and (ii) that the two compounds act through independent neurological mechanisms. The net reduction in UDysRS total score in the NLX-112 group (3.9 points more than placebo at day 42) is similar to that reported for a relatively short treatment (4-week) with amantadine (Goetz C G et al., “Which dyskinesia scale best detects treatment response?” Mov Disord 2013, 28, 341-6), suggesting that the clinical efficacy of the two compounds may be comparable.

Unlike previous serotonin 5-HT_{1A} agonists (see Introduction), the anti-LID activity of NLX-112 was not accompanied by a loss of antiparkinsonian activity of L-DOPA. Instead, NLX-112 elicited a further reduction of parkinsonism, as measured by UPDRS. Thus, patients on NLX-112 experienced a reduction in PD symptoms between baseline and days 28 and 42, based on patient- and Investigator-reported measures using the UPDRS (total and Part 3 scores; Table 22, FIGS. 3 and 4). Similarly, 4 of the 5 patients on NLX-112 and amantadine showed numerical reductions in PD symptoms as assessed using the UPDRS (Table 23). The reduction by NLX-112 of UPDRS Part 3 scores is particularly notable because it occurs after administration of a challenge dose of L-DOPA, i.e., when the patients were ‘ON’. This suggests that activation of 5-HT_{1A} receptors by NLX-112 elicits a further reduction of parkinsonism through a different mechanism to that engaged by dopamine replacement therapy. Moreover, the magnitude of the reduction in UPDRS Part 3 scores by NLX-112 (3.9 points more than placebo at day 42), is comparable to that achieved by dopamine agonists such as pramipexole, ropinirole or rotigotine (Thorlund K et al., “Nonergot dopamine-receptor agonists for treating Parkinson's disease—a network meta-analysis,” Neuropsychiatr Dis Treat 2014, 10, 767-76), suggesting that the effects of NLX-112 are clinically meaningful. In line with these findings, a greater numerical improvement in overall PD symptoms, as assessed by the CGI-C, was observed in the NLX-112 group as compared to the placebo group on day 42. Previous animal experiments have shown that NLX-112 increases rotation behavior in 6-OH-DA lesioned rats (Iderberg H et al., “NLX-112, a novel 5-HT1A receptor agonist for the treatment of L-DOPA-induced dyskinesia: Behavioral and neurochemical profile in rat,” Exp Neurol 2015, 271, 335-350) and reduces disability scores in MPTP-treated marmosets (Fisher R et al., “The selective 5-HT1A receptor agonist, NLX-112, exerts anti-dyskinetic and anti-parkinsonian-like effects in MPTP-treated marmosets,” Neuropharmacology 2020, 167, 107997), but the present observations are the first to show that such anti-parkinsonian activity translates to a clinical level for a selective 5-HT_{1A} receptor agonist.

In animal experiments, NLX-112 shows pronounced activity in models of pain, in a classic test of antidepressant-like activity (the forced swim test) and in improving urinary function in a spinal cord injury model (Colpaert FC, “5-HT(1A) receptor activation: new molecular and neuroadaptive mechanisms of pain relief,” Curr Opin Investig Drugs 2006, 7, 40-7; Iderberg et al., 2015; Lin C Y et al, “Improvement of lower urinary tract function by a selective serotonin 5-HT1A receptor agonist, NLX-112, after chronic spinal cord injury,” Exp Neurol 2020, 332, 113395; Newman-Tancredi, A., et al., “NLX-112, a highly selective 5-HT1A receptor agonist, mediates analgesia and antidepressant-like activity in rats via spinal cord and prefrontal cortex 5-HT1A receptors, respectively,” Brain Res 2018, 1688, 1-7), suggesting that it could also have activity against non-motor symptoms of PD. Here, positive effects were observed for both the NLX-112 group and the placebo group in terms of pain reduction evaluated using the KPPS, while there were no statistically significant effects in either treatment group in measures of anxiety or depression (assessed by HADS), quality of life (PDQ39) incontinence (ICIQ-OAB, significant negative effect within the placebo group on Day 28) or sleep (ESS) (Table 25). However, patient inclusion criteria were not based on these parameters and their basal values were generally low. Consequently, detection of potential efficacy of NLX-112 on non-motor symptoms requires trials conducted in populations of patients selected on the basis of higher basal symptom severity.

The anti-LID activity of NLX-112 likely involves activation of raphe 5-HT_{1A} autoreceptors and the consequent inhibition of serotonin neuron activity. This is supported by experiments in a parkinsonian (i.e., 6-OH-DA lesioned) rat model of LID, where NLX-112 inhibited striatal 5-HT release and blunted the ‘dopamine surge’ following L-DOPA administration (Iderberg et al., 2015). In contrast, the mechanisms underlying the antiparkinsonian effects of NLX-112 require further investigation. One possibility is that NLX-112 acts on 5-HT_{1A} receptors in striatal brain regions, as suggested by pre-clinical and clinical PET brain imaging using 18F-NLX-112 (Courault P et al., “[(18)F]F13640: a selective agonist PET radiopharmaceutical for imaging functional 5-HT(1A) receptors in humans,” Eur J Nucl Med Mol Imaging 2023, 50, 1651-1664; Vidal B et al, “[18F]-F13640 preclinical evaluation in rodent, cat and primate as a 5-HT1A receptor agonist for PET neuroimaging,” Brain Struct Funct 2018, 223, 2973-2988). In those studies, ¹⁸F-NLX-112 consistently labelled striatal 5-HT1A receptors in striatum of several species (rat, cat, macaque and human). Moreover, other studies have shown that 5-HT_{1A} receptors in rat striatum are active and can mediate antidyskinetic activity (Bishop C et al, “Contribution of the striatum to the effects of 5-HT1A receptor stimulation in L-DOPA-treated hemiparkinsonian rats,” Journal of Neuroscience Research 2009, 87, 1645-58; Meadows S M et al., “Characterizing the differential roles of striatal 5-HT1A auto- and hetero-receptors in the reduction of 1-DOPA-induced dyskinesia,” Exp Neurol 2017, 292, 168-178), suggesting that both raphe and striatal mechanisms may be involved in the effects of NLX-112. Functional brain imaging and EEG investigations could shed light on this (Brys I et al, “Neurophysiological effects in cortico-basal ganglia-thalamic circuits of antidyskinetic treatment with 5-HT1A receptor biased agonists,” Exp Neurol 2018, 302, 155-168; Chaib S et al, “Multimodal imaging study of the 5-HT(1A) receptor biased agonist, NLX-112, in a model of L-DOPA-induced dyskinesia,” Neuroimage Clin 2023, 39, 103497), particularly in the context of the ‘biased agonist’ profile of NLX-112 and related compounds. Indeed, NLX-112 preferentially activates specific cellular signaling cascades (via Gao G-proteins) (Newman-Tancredi et al., 2017) and shows accentuated activity in brain regions associated with motor control (Newman-Tancredi A et al, “Translating biased agonists from molecules to medications: Serotonin 5-HT1A receptor functional selectivity for CNS disorders,” Pharmacol Ther 2022, 229, 107937). Such biased agonist properties may underlie the superior profile of NLX-112 compared with previous serotonergic drugs.

Whilst this study points to favorable safety, tolerability and efficacy of NLX-112 in PD-LID patients, the study was conducted in a relatively small cohort of subjects, and its results are exploratory. Indeed, although motor-related symptoms of the patients in the NLX-112 group generally improved compared to baseline, statistically superior efficacy compared to the placebo group was not observed for most parameters (α=0.05). Part of the reason is that some improvement, although not statistically significant, was also observed for most motor-related aspects in the placebo group, which suggests that a placebo-effect had occurred. Moreover, the p-values for post-baseline vs. baseline comparisons within the placebo group are expected to be higher (a priori) than in the active group, since the sample size in the placebo group was half of that of the active treatment group. Hence, demonstration of statistically significant superiority of NLX-112 vs. placebo requires an appropriately powered study with a substantially larger sample size which takes the apparent placebo effect into account.

NLX-112 up-titrated over 4 weeks to a fixed dose of up to 2 mg/day, maintained at that dose for a further 2 weeks and down-titrated over 2 weeks was safe and well tolerated by most PD-LID patients as assessed by reported AEs, ECGs, vital signs, safety laboratory parameters, physical examinations and C-SSRS. Moreover, NLX-112 significantly reduced both LID and overall PD symptoms, suggesting that it could be a promising drug candidate for dual treatment of PD-LID and of PD symptoms. In fact, this is the first example of a drug that targets the 5-HT system and shows both anti-LID and anti-parkinsonian activity, supporting further development of the compound for treatment of PD and other movement disorders.

### Example 2—Sustained Release Oral Pharmaceutical Composition of Befiradol

WO 2016/005527 discloses methods of preparing sustained release oral pharmaceutical compositions of befiradol that can be used to prepare oral pharmaceutical compositions suitable for use in the methods of the present disclosure. For example, suitable sustained release, including once-daily, oral capsules may be prepared as outlined in Tables 26-28.

TABLE 26
Sustained Release Oral Capsule Compositions of Befiradol (2 mg)

| | SR1 Composition | | SR2 Composition | | SR3 Composition | |
|---|---|---|---|---|---|---|
| | Quantity | | Quantity | | Quantity | |
| Component | mg/capsule | %² | mg/capsule | %² | mg/capsule | %² |
| Befiradol fumarate | 2.60 | 1.23 | 2.60 | 1.17 | 2.60 | 1.11 |
| Equivalent to befiradol | 2.0 | | 2.0 | | 2.0 | |
| Sugar spheres (500-600 | 188.4 | 89.44 | 188.4 | 84.61 | 188.4 | 80.27 |
| μm) | | | | | | |
| Hydroxypropylcellulose | 9.0 | 4.27 | 9.0 | 4.04 | 9.0 | 3.83 |
| Surelease-clear E-7- | 10.0 | 4.75 | 22.0 | 9.88 | 34.0 | 14.49 |
| 19040 ®¹ | | | | | | |
| Talc | 0.64 | 0.30 | 0.68 | 0.31 | 0.72 | 0.31 |
| Total Fill Weight | 210.64 | 100 | 222.68 | 100 | 234.72 | 100 |
| Hard Capsule³ | 1 | | 1 | | 1 | |

¹Expressed as dry material corresponding to about 25% of aqueous Surelease dispersion; composition of Surelease E-7-19040 ® (available from Colorcon): purified water, ethyl cellulose 20 mPa · s, ammonium hydroxide 28%, triglycerides medium-chain, oleic acid.
²Expressed as dry weight, of the total weight of the composition
³Hard Capsule shell composition (% w/w): Red iron oxide (0.47%), titanium dioxide (1.0%), yellow iron oxide (0.45%), gelatin (qsp 100%).

TABLE 27
Sustained Release Oral Capsule Compositions of Befiradol (2.5 mg)

| | SR4 Composition | | SR5 Composition | | SR6 Composition | |
|---|---|---|---|---|---|---|
| | Quantity | | Quantity | | Quantity | |
| Component | mg/capsule | %² | mg/capsule | %² | mg/capsule | %² |
| Befiradol fumarate | 3.25 | 1.23 | 3.25 | 1.17 | 3.25 | 1.11 |
| Equivalent to befiradol | 2.5 | | 2.5 | | 2.5 | |
| Sugar spheres (500-600 | 235.5 | 89.44 | 235.5 | 84.61 | 235.5 | 80.27 |
| μm) | | | | | | |
| Hydroxypropylcellulose | 11.25 | 4.27 | 11.25 | 4.04 | 11.25 | 3.83 |
| Surelease-clear E-7- | 12.5 | 4.75 | 27.5 | 9.88 | 42.5 | 14.49 |
| 19040 ®¹ | | | | | | |
| Talc | 0.8 | 0.30 | 0.85 | 0.31 | 0.9 | 0.31 |
| Total Fill Weight | 263.3 | 100 | 278.35 | 100 | 293.4 | 100 |
| Hard Capsule³ | 1 | | 1 | | 1 | |

¹Expressed as dry material corresponding to about 25% of aqueous Surelease dispersion; composition of Surelease E-7-19040 ® (available from Colorcon): purified water, ethyl cellulose 20 mPa · s, ammonium hydroxide 28%, triglycerides medium-chain, oleic acid.
²Expressed as dry weight, of the total weight of the composition
³Hard Capsule shell composition (% w/w): Red iron oxide (0.47%), titanium dioxide (1.0%), yellow iron oxide (0.45%), gelatin (qsp 100%).

TABLE 28
Sustained Release Oral Capsule Compositions of Befiradol (3 mg)

| | SR7 Composition | | SR8 Composition | | SR9 Composition | |
|---|---|---|---|---|---|---|
| | Quantity | | Quantity | | Quantity | |
| Component | mg/capsule | %² | mg/capsule | %² | mg/capsule | %² |
| Befiradol fumarate | 3.9 | 1.23 | 3.9 | 1.17 | 3.9 | 1.11 |
| Equivalent to befiradol | 3.0 | | 3.0 | | 3.0 | |
| Sugar spheres (500-600 | 282.6 | 89.44 | 282.6 | 84.61 | 282.6 | 80.27 |
| μm) | | | | | | |
| Hydroxypropylcellulose | 13.5 | 4.27 | 13.5 | 4.04 | 13.5 | 3.83 |
| Surelease-clear E-7- | 15.0 | 4.75 | 33.0 | 9.88 | 51.0 | 14.49 |
| 19040 ®¹ | | | | | | |
| Talc | 0.96 | 0.30 | 1.02 | 0.31 | 1.08 | 0.31 |
| Total Fill Weight | 315.96 | 100 | 334.02 | 100 | 352.08 | 100 |
| Hard Capsule³ | 1 | | 1 | | 1 | |

¹Expressed as dry material corresponding to about 25% of aqueous Surelease dispersion; composition of Surelease E-7-19040 ® (available from Colorcon): purified water, ethyl cellulose 20 mPa · s, ammonium hydroxide 28%, triglycerides medium-chain, oleic acid.
²Expressed as dry weight, of the total weight of the composition
³Hard Capsule shell composition (% w/w): Red iron oxide (0.47%), titanium dioxide (1.0%), yellow iron oxide (0.45%), gelatin (qsp 100%).

Based on the befiradol Cₘₐₓ values observed in healthy male volunteers reported in WO 2016/005527, the sustained release compositions disclosed above in Tables 26-28 would be expected to provide Cₘₐₓ values not less than those shown in Table 29 after a single oral administration to patients with Parkinson's disease. Note in this regard that based on unpublished data, female subjects can exhibit befiradol Cₘₐₓ about 20%-40% higher than males, and elderly subjects can exhibit befiradol Cₘₐₓ higher than young subjects. Therefore, the Cₘₐₓ values in Table 29 for SR1-SR9 may be higher (e.g., by 10% or 20%) when administered to patients with Parkinson's disease since young adults rarely experience Parkinson's disease, which usually develops around age 60 or older.

TABLE 29
Projected Cₘₐₓ After Single Oral Administration

| Composition | Befiradol Dose (mg) | Cₘₐₓ (ng/mL) |
|---|---|---|
| MR1¹ | 1^{1, 2} | 18.2¹ |
| MR2¹ | 1^{1, 2} | 11.4¹ |
| MR3¹ | 1^{1, 2} | 9.9¹ |
| SR1 | 2 | 36³ |
| SR2 | 2 | 23⁴ |
| SR3 | 2 | 20⁵ |
| SR4 | 2.5 | 46³ |
| SR5 | 2.5 | 29⁴ |
| SR6 | 2.5 | 25⁵ |
| SR7 | 3 | 55³ |
| SR8 | 3 | 34⁴ |
| SR9 | 3 | 30⁵ |

¹WO 2016/005527 Example 3
²2 × 0.5 mg capsules
³(18.2 × befiradol dose)
⁴(11.4 × befiradol dose)
⁵(9.9 × befiradol dose)

### Example 3—Phase 1 Study of the Pharmacokinetics of NLX-112 Capsules in Male and Female Elderly Volunteers

### Objectives

Primary: To assess the pharmacokinetics of NLX-112 when administered as a single dose of 2, 2.5 or 3 mg to healthy elderly volunteers.

Secondary: To assess the clinical and biological tolerability of a single oral dose of NLX-112 when administered to healthy elderly volunteers.

### Methodology

Single center, open label, single dose.

Blood samples for befiradol plasma level determinations are collected at the following times: T0 (before drug administration), T0.25 h, T0.5 h, T0.75 h, T1 h, T1.5 h, T2 h, T3 h, T4 h, T5 h, T6 h, T8 h, T12 h, T24 h (Day 2), T48 h (Day 3), T72 h (Day 4), T96 h (Day 5), T120 h (Day 6) and T144 h (Day 7) after drug administration.

### Number of Subjects

Twelve (12) elderly volunteers, 6 males and 6 females.

### Diagnosis and Main Criteria for Inclusion/Exclusion

### Inclusion Criteria

To be included in the study, subjects must fulfill all the following criteria:
Healthy male or female Caucasian volunteer aged ≥65 years
Normal clinical examination, compatible with the study in the investigator's opinion.
Body Mass Index (BMI) score between 18 and 30 kg/m2.
Absence of current psychiatric disorders.
Routine laboratory safety tests, normal for this elderly population
Healthy Negative serology for Hbs antigens, HCV and HIV 1 and 2 antibodies.
Normal ECG and normal vital signs or considered as non-significant with regard to this elderly population.

### Exclusion Criteria

Study participants will not be entered in the study if any of the following apply:
Any medically significant history, or presence of neurologic disease, cancer, cardiac, respiratory, metabolic, hepatic, renal, gastrointestinal (except appendectomy), dermatological, venereal, haematological disorder or disease.
Volunteer who had demonstrated intolerance or hypersensitivity to NLX-112, or related drugs, in previous clinical studies, or history of relevant allergic reaction of any origin.
Volunteer who smoked.
History of alcoholism and/or drug addiction and/or positive findings on either urinary drug screening or alcohol test.
Consumption of large quantities (more than 6 cups per day) of xanthine-containing drinks (coffee, tea, cola) and who are unable to abstain for the duration of the study.
Volunteer who had received any non-authorized treatment within 21 days before the study drug administration.
Treatment intake which could have led to induction or inhibition of hepatic microsomal enzymes, within 2 months of the study drug administration.
Regular intensive sports activities or unable to abstain from intensive muscular effort and/or sports competitions throughout the study.
Volunteer who could not be contacted in case of emergency.
Volunteer who had donated blood in the two months preceding the study.
Volunteer who had forfeited his freedom by administrative or legal award or who is under guardianship.

Additionally, a volunteer who, in the investigator or sponsor's opinion, would be unlikely to comply with the protocol or co-operate during the study itself will not be included.

Medication intake (except hormone replacement therapy for women and lipid lowering drugs) within 21 days prior to the study drug administration excluded the possibility of inclusion into the study. Intake of any medication is not allowed for the duration of the study, except hormone replacement therapy for women and lipid lowering drugs. Intake of treatment which could lead to induction or inhibition of hepatic microsomal enzymes, within 2 months of the first study drug administration, is not allowed.

If a corrective treatment is required for an adverse event, the investigators have the right to prescribe it. The following information is to be recorded in the appropriate pages of the CRF:
The reason for treatment,
The time and date of start,
The name of the treatment and its presentation,
The route of administration,
The daily dosage given,
The duration of treatment.

### Test Product, Dose and Mode of Administration

NLX-112 capsules described in Example 2, above, single oral administration in fasting conditions with 140 mL of mineral water after an overnight fast of at least 10 hours around 8:00 am on Day 1.

### Duration of Treatment

Single administration.

### Criteria for Evaluation

### Main Criteria: Pharmacokinetics:

Blood Samples: 19 samples, from T₀ to T₁₄₄ₕ (Day 7)
Bioanalysis of NLX-112 in plasma with LC/MS-MS method (LOQ of 0.1 ng/ml)

Secondary criteria: Tolerability: Clinical examination, vital signs, ECG, AEs recording, blood and urine for routine safety laboratory parameters.

### Statistical Methods

Pharmacokinetics: Descriptive statistics on the main PK parameters (mean, median, SD, CV, min and max).

Safety: Descriptive analyses of safety volunteer data set.

### Example 4—Study of the Tolerability and Pharmacokinetics of NLX-112 Capsules, Administered Daily for 14 Days

### Objectives

Primary: To assess the clinical and laboratory safety of NLX-112, compared with placebo, and determine the MTD after a 14-day repeated oral dosing in healthy volunteers; to carry out a pharmacokinetic (PK) assessment of single and repeated oral doses of NLX-112.

### Methodology

Single center, double-blinded, repeated-dose study in sequential ascending level (1.5, 2, 2.5, 3 mg/day) groups of healthy volunteers.

### Number of Subjects

64 subjects in 4 level groups of 16 subjects each (12 NLX-112, 4 placebo); all are analyzed for safety and PK.

### Diagnosis and Main Criteria for Inclusion/Exclusion

### Inclusion Criteria

To be included in the study, subjects must fulfill all the following criteria:
Healthy male or female Caucasian volunteer aged 18-45 years
Normal clinical examination, compatible with the study in the investigator's opinion.
Body Mass Index (BMI) score between 18 and 28 kg/m2 and weight ≤90 kg.
Absence of current psychiatric disorders.
Normal personality in the investigator's opinion.
Normal or considered non clinically significant routine laboratory safety tests (within 1.5× upper limit of normality -ULN- for aspartate aminotransferase -AST- and alanine aminotransferase -ALT-).
Negative serology for hepatitis B antigen (Hbs), anti-hepatitis C virus -HCV-antibodies and anti-human immunodeficiency virus -HIV- 1 and 2 antibodies.
Normal ECG (in particular, QT corrected for HR according to Bazett's formula -QT_{CB}-≤430 ms) and vital signs (100 mmHg≤systolic blood pressure -SBP-≤150 mmHg; 45 mmHg≤diastolic blood pressure -DBP-≤90 mm Hg; 45 beats per minute -bpm-≤heart rate -HR-≤100 bpm).

### Exclusion Criteria

Study participants will not be entered in the study if any of the following apply:
Known renal impairment.
Known hepatic impairment.
Known Raynaud syndrome.
History of drug allergy or current allergic reaction.
History of prolonged QT interval.
History of hypo- or hypertension.
Intake of psychoactive drugs within 3 weeks prior to first dosing.
Intake of hepatic inducers or inhibitors or any medicines containing analgesics, corticosteroids, NSAIDs, antidepressants, anticonvulsivants or neuroleptics within 4 weeks prior to first dosing.
Smoker.
Previous or current history of temporary or chronic alcohol abuse.
Positive alcohol breath test.
Previous or current history of drug addiction.
Positive urine drug screen.
Consumption of large quantities (more than 6 cups per day) of xanthine-containing drinks (coffee, tea, cola) and who are unable to abstain for the duration of the study.
Volunteer who had received any non-authorized treatment within 21 days before the study drug administration.
Practicing regular intensive sports activities and/or unable to abstain from intensive muscular effort and/or sport competitions throughout the study.
Volunteer who could not be contacted in case of emergency.
Volunteer who had donated blood in the two months preceding the study.
Volunteer who had forfeited his freedom by administrative or legal award or who is under guardianship.
Who, in the opinion of investigator or sponsor, is unlikely to comply with the protocol or cooperate during the study itself.

### Test Product, Dose and Mode of Administration

NLX-112 capsules analogous to those described in Example 2, above, and matching placebo capsules. 4 dose groups: 1.5, 2, 2.5 and 3 mg once a day (qd). Oral admin in fasting conditions after an overnight fast of at least 10 hours.

### Duration of Treatment

14 days (+7±2 days of post-treatment observation).

### Main Criteria for Evaluation

### Safety:

Adverse events: continuous reporting from Selection visit (between Day −21 and Day −3) to End of Study visit.
Vital Signs SBP, DBP and HR of Day −1 (T−1 h), on Day 1 and Day 14 (T−1 h, T0.5 h, T1 h, T2 h, T3 h, T4 h, T5 h, T6 h, T7 h, T8 h, T10 h, and T12 h, from Day 2 to Day 13 (T−1 h and T1 h), on Day 15 (T−1 h, T1h and T2.5 h), on Day 21 (in the morning); Respiratory rate: on Days 1, 4, 5 and 13 (T−1 h and T1 h), on Day 14 (T−1 h).
ECG: QT_{CB} and QT_{CF}: on Day −1 (T−1 h), on Days 1, 2, 4, 6, 9, 11 and 13 (T−1 h, T1h and T3 h), on Day 15 (T1 h), on Day 21 (morning).
Standard lab tests: hematology, biochemistry and urinalysis: on Day −1 (morning), on Days 3, 7, 9, 12 and 15 (T−1 h), on Day 21 (morning).

### Pharmacokinetics:

Plasma samples: on Days 1 and 14 (T0 -before dosing-, T0.5 h, T1 h, T2 h, T3 h, T4 h, T5 h, T6 h, T7 h, T8 h, T12h and T24h=T0 of Days 2 and 15), on Days 3, 5, 7, 9, 11, and 13 (T0).
Parameters: Cₘₐₓ, Tₘₐₓ, AUC₀₋₂₄ₕ, Cₘᵢₙ

### Statistical Methods

Pharmacokinetics: Descriptive statistics, graphical approach for Cₘᵢₙ evolution and for evaluation of linearity with dose of PK parameters.

### Safety:

Adverse events: frequencies, tabulated individual data
Vital signs: descriptive statistics for values and changes, frequencies of FDA predefined clinically relevant changes and clinically significant changes (PC and CSC, respectively) including orthostatic hypotension
ECG/QTCB and QTCF: Descriptive statistics for values and changes, frequencies by CPMP classes
Standard lab tests: Descriptive statistics for values and changes, frequencies of shifts/normal range, frequencies and tabulated individual data for predefined clinically relevant changes and clinically noteworthy abnormal lab values (CNALV)
Concomitant treatments: frequencies by ATC classes.

### Example 5—Study of the Tolerability and Pharmacokinetics of NLX-112 Capsules, Administered Daily for 14 Days to Male and Female Elderly Volunteers

### Objectives

Primary: To assess the clinical and laboratory safety of NLX-112, compared with placebo, and determine the MTD after a 14-day repeated oral dosing in healthy elderly volunteers; to carry out a pharmacokinetic (PK) assessment of single and repeated oral doses of NLX-112.

### Methodology

Single center, double-blinded, repeated-dose study in sequential ascending level (1.5, 2, 2.5, 3 mg/day) groups of healthy elderly volunteers.

### Number of Subjects

64 subjects, 32 males and 32 females, in 4 level groups of 16 subjects each (6 males and 6 females, NLX-112; 2 males and 2 females, placebo); all are analyzed for safety and PK.

### Diagnosis and Main Criteria for Inclusion/Exclusion

### Inclusion Criteria

To be included in the study, subjects must fulfill all the following criteria:
Healthy male or female Caucasian volunteer aged ≥65 years
Normal clinical examination, compatible with the study in the investigator's opinion.
Body Mass Index (BMI) score between 18 and 30 kg/m².
Absence of current psychiatric disorders.
Routine laboratory safety tests, normal for this elderly population
Healthy Negative serology for Hbs antigens, HCV and HIV 1 and 2 antibodies.
Normal ECG and normal vital signs or considered as non-significant with regard to this elderly population.

### Exclusion Criteria

Study participants will not be entered in the study if any of the following apply:
Any medically significant history, or presence of neurologic disease, cancer, cardiac, respiratory, metabolic, hepatic, renal, gastrointestinal (except appendectomy), dermatological, venereal, haematological disorder or disease.
Volunteer who had demonstrated intolerance or hypersensitivity to NLX-112, or related drugs, in previous clinical studies, or history of relevant allergic reaction of any origin.
Current smoker.
History of alcoholism and/or drug addiction and/or positive findings on either urinary drug screening or alcohol test.
Consumption of large quantities (more than 6 cups per day) of xanthine-containing drinks (coffee, tea, cola) and who are unable to abstain for the duration of the study.
Volunteer who had received any non-authorized treatment within 21 days before the study drug administration.
Treatment intake which could have led to induction or inhibition of hepatic microsomal enzymes, within 2 months of the study drug administration.
Regular intensive sports activities or unable to abstain from intensive muscular effort and/or sports competitions throughout the study.
Volunteer who could not be contacted in case of emergency.
Volunteer who had donated blood in the two months preceding the study.
Volunteer who had forfeited his freedom by administrative or legal award or who is under guardianship.

Additionally, a volunteer who, in the investigator or sponsor's opinion, would be unlikely to comply with the protocol or co-operate during the study itself will not be included.

Medication intake (except hormone replacement therapy for women and lipid lowering drugs) within 21 days prior to the study drug administration excluded the possibility of inclusion into the study. Intake of any medication is not allowed for the duration of the study, except hormone replacement therapy for women and lipid lowering drugs. Intake of treatment which could lead to induction or inhibition of hepatic microsomal enzymes, within 2 months of the first study drug administration, is not allowed.

If a corrective treatment is required for an adverse event, the investigators have the right to prescribe it. The following information is to be recorded in the appropriate pages of the CRF:
The reason for treatment,
The time and date of start,
The name of the treatment and its presentation,
The route of administration,
The daily dosage given,
The duration of treatment.

### Test Product, Dose and Mode of Administration

NLX-112 capsules analogous to those described in Example 2, above, and matching placebo capsules. 4 dose groups: 1.5, 2, 2.5 and 3 mg once a day (qd). Oral admin in fasting conditions after an overnight fast of at least 10 hours.

### Duration of Treatment

14 days (+7±2 days of post-treatment observation).

### Main Criteria for Evaluation

### Safety:

Adverse events: continuous reporting from Selection visit (between Day −21 and Day −3) to End of Study visit.
Vital Signs SBP, DBP and HR of Day −1 (T−1 h), on Day 1 and Day 14 (T−1 h, T0.5 h, T1 h, T2 h, T3 h, T4 h, T5 h, T6 h, T7 h, T8 h, T10 h, and T12 h, from Day 2 to Day 13 (T−1h and T1 h), on Day 15 (T−1 h, T1h and T2.5 h), on Day 21 (in the morning); Respiratory rate: on Days 1, 4, 5 and 13 (T−1h and T1 h), on Day 14 (T−1 h).
ECG: QT_{CB} and QT_{CF}: on Day−1 (T−1 h), on Days 1, 2, 4, 6, 9, 11 and 13 (T−1 h, T1h and T3 h), on Day 15 (T1 h), on Day 21 (morning).
Standard lab tests: hematology, biochemistry and urinalysis: on Day −1 (morning), on Days 3, 7, 9, 12 and 15 (T−1 h), on Day 21 (morning).

### Pharmacokinetics:

Plasma samples: on Days 1 and 14 (T0 -before dosing-, T0.5 h, T1 h, T2 h, T3 h, T4 h, T5 h, T6 h, T7 h, T8 h, T12h and T24h=T0 of Days 2 and 15), on Days 3, 5, 7, 9, 11, and 13 (T0).
Parameters: Cₘₐₓ, Tₘₐₓ, AUC₀₋₂₄ₕ, Cₘᵢₙ

### Statistical Methods

Pharmacokinetics: Descriptive statistics, graphical approach for Cₘᵢₙ evolution and for evaluation of linearity with dose of PK parameters.

### Safety:

Adverse events: frequencies, tabulated individual data
Vital signs: descriptive statistics for values and changes, frequencies of FDA predefined clinically relevant changes and clinically significant changes (PC and CSC, respectively) including orthostatic hypotension
ECG/QT_{CB} and QT_{CF}: Descriptive statistics for values and changes, frequencies by CPMP classes
Standard lab tests: Descriptive statistics for values and changes, frequencies of shifts/normal range, frequencies and tabulated individual data for predefined clinically relevant changes and clinically noteworthy abnormal lab values (CNALV)
Concomitant treatments: frequencies by ATC classes.

### Example 6—Conventional Immediate Release Tablet of Befiradol Fumarate

The conventional immediate release tablets used in the Phase 2a clinical trial described in Example 1 had the composition set forth in Table 30.

TABLE 30
Drug Product Composition

| | Quantity | Quantity | | |
|---|---|---|---|---|
| Component | (mg/tablet) | (g/batch)¹ | Percent | Function |
| NLX-112 fumarate salt | 0.33² | 11.98 | 0.33² | Active |
| | | | | ingredient |
| Calcium phosphate, dibasic, | 59.57 | 2162.39 | 59.57 | Filler |
| anhydrous | | | | |
| Microcrystalline cellulose | 39.50 | 1433.85 | 39.50 | Filler |
| Colloidal silicon dioxide, anhydrous | 0.10 | 3.63 | 0.10 | Glidant |
| Magnesium stearate | 0.50 | 18.15 | 0.50 | Lubricant |
| Total | 100 | 3630.00 | 100 | |

¹This batch size yields approximately 36,300 tablets
²Equivalent to 0.25 mg (0.25%) NLX-112 base. The drug substance is adjusted based on 99% purity

### Weighing

NLX-112 fumarate salt was screened though a US Standard #60 mesh screen and the required amount for the batch weighed. The calcium phosphate dibasic anhydrous, microcrystalline cellulose, colloidal silicon dioxide, and magnesium stearate for the batch were weighed. The screened NLX-112 fumarate salt was transferred into a bag (capable of holding approximately 500 g of material) containing the colloidal silicon dioxide. The microcrystalline cellulose was screened though a US Standard #30 mesh screen and about 75-100 g was added into the bag, the bag was closed, and the contents manually mixed 50 times. Another ca. 300 g of screened microcrystalline cellulose was added to the bag and the contents manually mixed 50 times. The mixed bag contents were then screened through a US Standard #30 mesh screen and collected into a double-lined polyethylene bag (API Bag Mix). The calcium phosphate dibasic was screened through a US Standard #30 mesh screen.

### Blending

The following screened materials were added into a 8 qt V-blender in the following order: 1) about 515 g of microcrystalline cellulose; 2) the API Bag Mix; 3) the remaining quantity of microcrystalline cellulose. The blender lid was closed and then mixed for about 5 minutes=0.5 minute. The screened calcium phosphate dibasic anhydrous was then added into the V-blender with approximately equal amounts into each arm. The blender lid was then closed and mixed for about 5 minutes±0.5 minute. The blend was discharged from the blender, screened though a #30 mesh screen, then loaded back into the blender and mixed for about 10 minutes.

### In-Process Blend Sampling Pre-Lubrication

Using a 1 ft. sampling thief, 10 samples were collected, in duplicate. The net weight of each sample was 150 mg-250 mg, target 200 mg. The weights were recorded to whole milligram (gross, tare, net). The samples were tested for blend uniformity. The acceptance criterion was that each individual sample assay should be within the range of 90.0 to 110.0%.

The magnesium stearate was screened through a US Standard #30 mesh screen. A cavity was made in the blended material (still in the blender) and the screened magnesium stearate was added. The magnesium stearate was covered with the blended material, then the lid was closed and mixed for about 5 minutes±0.5 minute. The blended material was then discharged from the V-blender.

### Tablet Press Set Up

A Picolla B-10 tablet press was set up along with tablet de-duster and metal detector in series. The compression tooling was set up:
Punch type used: 0.1969 inch-round-no embossing (Type B tooling)
SC die type used: 0.1969″
Number of stations: 10
Main compression: ˜3.0 (range 2.0-3.5)
Pre-compression: no (range: 0 to 20%)
Force feeder speed dial setting: 4 (range 3-5)
Product speed dial setting: 4 (range 3-5)

### Tablet Production

The blend was fed into the hopper of the tablet press and compressed into tablets.
Target tablet weight=100 mg (acceptable range: 93 mg-107 mg)
Target tablet thickness=3.65 mm (acceptable range: 3.50 mm-3.80 mm)
Tablet hardness: not less than 8.0 kP
Friability: not more than 1.00% after 100 rotations (at the start of the compression)
Disintegration time: not more than 15 minutes (at the start of the compression)

Manufactured tablets were collected in a container while visually inspecting bulk tablets for defects. Manufactured tablets were segregated into individual bags marked with the bag number every 15 minutes or after machine adjustments affecting weight, hardness, or thickness. Compression was stopped once the required production quantity is met.

Tablet in-Process Testing

10 individual tablets were randomly selected from each bag and visually inspected. Tablet weight, thickness, and hardness was tested. Tested in-process tablet remains were rejected and kept for reconciliation purposes.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method for treating L-DOPA-induced dyskinesia in a patient in need thereof, comprising daily administering to the patient a therapeutically effective daily dosage of L-DOPA and befiradol or a pharmaceutically acceptable salt thereof, wherein:
a. the method is safe and well tolerated;
b. the therapeutically effective daily dosage of befiradol or a pharmaceutically acceptable salt thereof provides, after at least two weeks of treatment according to the method, a mean maximum befiradol blood plasma concentration of at least about 30 ng/mL; and
c. the therapeutically effective daily dosage of befiradol or a pharmaceutically acceptable salt thereof is administered in a sustained release pharmaceutical composition.

2. A method for treating L-DOPA-induced dyskinesia in a patient in need thereof, comprising daily administering to the patient a therapeutically effective daily dosage of L-DOPA and befiradol or a pharmaceutically acceptable salt thereof, wherein:
a. the method is safe and well tolerated;
b. the therapeutically effective daily dosage of befiradol or a pharmaceutically acceptable salt thereof provides, after at least two weeks of treatment according to the method, a mean maximum befiradol blood plasma concentration of at least about 30 ng/ml; and
c. the therapeutically effective daily dosage of L-DOPA and befiradol or a pharmaceutically acceptable salt thereof is administered to the patient daily for at least 6 weeks.

3. A method for treating L-DOPA-induced dyskinesia in a patient in need thereof, comprising daily administering to the patient a therapeutically effective daily dosage of L-DOPA and befiradol or a pharmaceutically acceptable salt thereof, wherein:
a. the method is safe and well tolerated;
b. the therapeutically effective daily dosage of befiradol or a pharmaceutically acceptable salt thereof provides, after at least two weeks of treatment according to the method, a mean maximum befiradol blood plasma concentration of at least about 30 ng/ml; and
c. the therapeutically effective daily dosage of befiradol or a pharmaceutically acceptable salt thereof is at least about 5.7 mmol.

4. The method of claim 1, wherein the method comprises a titration phase followed by a treatment phase, wherein:
a. at the beginning of the titration phase the befiradol or a pharmaceutically acceptable salt thereof is administered at an initial daily dosage that is lower than the therapeutically effective daily dosage of befiradol or a pharmaceutically acceptable salt thereof;
b. during the titration phase the initial daily dosage of befiradol or a pharmaceutically acceptable salt thereof is gradually increased to the therapeutically effective daily dosage of befiradol or a pharmaceutically acceptable salt thereof; and
c. during the treatment phase the therapeutically effective daily dosage of L-DOPA and befiradol or a pharmaceutically acceptable salt thereof is administered daily.

5. The method of claim 2, wherein the method comprises a titration phase followed by a treatment phase, wherein:
a. at the beginning of the titration phase the befiradol or a pharmaceutically acceptable salt thereof is administered at an initial daily dosage that is lower than the therapeutically effective daily dosage of befiradol or a pharmaceutically acceptable salt thereof;
b. during the titration phase the initial daily dosage of befiradol or a pharmaceutically acceptable salt thereof is gradually increased to the therapeutically effective daily dosage of befiradol or a pharmaceutically acceptable salt thereof; and
c. during the treatment phase the therapeutically effective daily dosage of L-DOPA and befiradol or a pharmaceutically acceptable salt thereof is administered daily.

6. The method of claim 3, wherein the method comprises a titration phase followed by a treatment phase, wherein:
a. at the beginning of the titration phase the befiradol or a pharmaceutically acceptable salt thereof is administered at an initial daily dosage that is lower than the therapeutically effective daily dosage of befiradol or a pharmaceutically acceptable salt thereof;
b. during the titration phase the initial daily dosage of befiradol or a pharmaceutically acceptable salt thereof is gradually increased to the therapeutically effective daily dosage of befiradol or a pharmaceutically acceptable salt thereof; and
c. during the treatment phase the therapeutically effective daily dosage of L-DOPA and befiradol or a pharmaceutically acceptable salt thereof is administered daily.

7. The method of claim 4, wherein the therapeutically effective daily dosage of befiradol or a pharmaceutically acceptable salt thereof is administered orally.

8. The method of claim 5, wherein the therapeutically effective daily dosage of befiradol or a pharmaceutically acceptable salt thereof is administered orally.

9. The method of claim 6, wherein the therapeutically effective daily dosage of befiradol or a pharmaceutically acceptable salt thereof is administered orally.

10. The method of claim 7, wherein the therapeutically effective daily dosage of befiradol or a pharmaceutically acceptable salt thereof provides, after at least two weeks of treatment according to the method, a mean maximum befiradol blood plasma concentration of at least about 50 ng/mL.

11. The method of claim 8, wherein the therapeutically effective daily dosage of befiradol or a pharmaceutically acceptable salt thereof provides, after at least two weeks of treatment according to the method, a mean maximum befiradol blood plasma concentration of at least about 50 ng/mL.

12. The method of claim 9, wherein the therapeutically effective daily dosage of befiradol or a pharmaceutically acceptable salt thereof provides, after at least two weeks of treatment according to the method, a mean maximum befiradol blood plasma concentration of at least about 50 ng/mL.

13. The method of claim 10, wherein the therapeutically effective daily dosage of befiradol or a pharmaceutically acceptable salt thereof is administered to the patient each day in one or more daily doses, and each such dose, if administered once to at least 6 males and at least 6 females in a clinical trial without a befiradol daily dosage up-titration period, provides a mean maximum plasma concentration of befiradol greater than about 15 ng/mL.

14. The method of claim 11, wherein the therapeutically effective daily dosage of befiradol or a pharmaceutically acceptable salt thereof is administered to the patient each day in one or more daily doses, and each such dose, if administered once to at least 6 males and at least 6 females in a clinical trial without a befiradol daily dosage up-titration period, provides a mean maximum plasma concentration of befiradol greater than about 15 ng/mL.

15. The method of claim 12, wherein the therapeutically effective daily dosage of befiradol or a pharmaceutically acceptable salt thereof is administered to the patient each day in one or more daily doses, and each such dose, if administered once to at least 6 males and at least 6 females in a clinical trial without a befiradol daily dosage up-titration period, provides a mean maximum plasma concentration of befiradol greater than about 15 ng/mL.

16. The method of claim 13, wherein the method does not significantly worsen the patient's parkinsonian symptoms.

17. The method of claim 14, wherein the method does not significantly worsen the patient's parkinsonian symptoms.

18. The method of claim 15, wherein the method does not significantly worsen the patient's parkinsonian symptoms.

19. The method of claim 13, wherein the method does not significantly worsen the patient's parkinsonian motor symptoms.

20. The method of claim 14, wherein the method does not significantly worsen the patient's parkinsonian motor symptoms.
